(19) United States
(12) Patent Application Publication
(10) Pub. No.: US 2019/0202799 A1
(43) Pub. Date: Jul. 4, 2019

(54) IMIDAZOLE DERIVATIVES AND THEIR USE IN THE TREATMENT OF AUTOIMMUNE OR INFLAMMATORY DISEASES OR CANCERS

(71) Applicant: GLAXOSMITHKLINE INTELLECTUAL PROPERTY (No.2) LIMITED, Brentford Middlesex (GB)

(72) Inventors: Andrew BAXTER, Stevenage, Hertfordshire (GB); Rino Antonio BIT, Stevenage, Hertfordshire (GB); John Alexander BROWN, Cambridge, MA (US); David HIRST, Stevenage, Hertfordshire (GB); Philip HUMPHREYS, Stevenage, Hertfordshire (GB); Katherine Louise JONES, Stevenage, Hertfordshire (GB); Vipulkumar Kantibhai PATEL, Stevenage, Hertfordshire (GB)

(21) Appl. No.: 16/327,016

(22) PCT Filed: Aug. 31, 2017
(86) PCT No.: PCT/EP2017/071892

§ 371 Date: Feb. 21, 2019

(30) Foreign Application Priority Data
Sep. 2, 2016 (GB) 1614940.3

(51) Int. Cl. C07D 401/04; C07D 401/14; C07D 413/14; C07D 405/14; A61P 29/00; A61P 37/02; A61P 19/02

(52) U.S. Cl. CPC C07D 401/04; C07D 401/14; C07D 413/14; A61P 19/02; A61P 29/00; A61P 37/02; C07D 405/14

(57) ABSTRACT

Compounds of formula (I) and salts thereof:

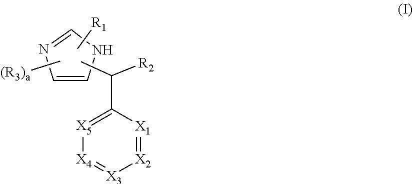

wherein R₁, R₂, R₃, a, X₁, X₂, X₃, X₄, and X₅ are as defined hereinbefore. Compounds of formula (I) and salts thereof have been found to inhibit the binding of the BET family of bromodomain containing proteins to, for example, acetylated lysine residues and thus may have use in therapy, for example in the treatment of autoimmune and inflammatory diseases, such as rheumatoid arthritis; and cancers.

### FIELD OF THE INVENTION

The present invention relates to compounds, compositions containing them, and to their use in the treatment of various disorders in particular inflammatory and autoimmune diseases, such as rheumatoid arthritis, and cancers.

### BACKGROUND TO THE INVENTION

The genomes of eukaryotic organisms are highly organised within the nucleus of the cell. The long strands of duplex DNA are wrapped around an octomer of histone proteins (most usually comprising two copies of histones H2A, H2B, H3 and H4) to form a nucleosome. This basic unit is then further compressed by the aggregation and folding of nucleosomes to form a highly condensed chromatin structure. A range of different states of condensation are possible, and the tightness of this structure varies during the cell cycle, being most compact during the process of cell division. Chromatin structure plays a critical role in regulating gene transcription, which cannot occur efficiently from highly condensed chromatin. The chromatin structure is controlled by a series of post translational modifications to histone proteins, notably histones H3 and H4, and most commonly within the histone tails which extend beyond the core nucleosome structure. These modifications include acetylation, methylation, phosphorylation, ubiquitinylation, and SUMOylation. These epigenetic marks are written and erased by specific enzymes, which place tags on specific residues within the histone tail, thereby forming an epigenetic code, which is then interpreted by the cell to allow regulation of gene expression.

Histone acetylation is most usually associated with the activation of gene transcription, as the modification relaxes the interaction of the DNA and the histone octomer by changing the electrostatics. In addition to this physical change, specific proteins recognise and bind to acetylated lysine residues within histones to read the epigenetic code. Bromodomains are small (˜110 amino acid) distinct domains within proteins that bind to acetylated lysine resides commonly but not exclusively in the context of histones. There is a family of around 50 proteins known to contain bromodomains, and they have a range of functions within the cell.

The BET family of bromodomain containing proteins comprises 4 proteins (BRD2, BRD3, BRD4 and BRDT) which contain tandem bromodomains capable of binding to two acetylated lysine residues in close proximity, increasing the specificity of the interaction. Numbering from the N-terminal end of each BET protein the tandem bromodomains are typically labelled Binding Domain 1 (BD1) and Binding Domain 2 (BD2) (Chung et al., J. Med. Chem., 2011, 54, 3827-3838).

Inhibiting the binding of a BET protein to acetylated lysine residues has the potential to ameliorate progression of several diseases, including but not limited to, cancer (Dawson M. A. et al., Nature, 2011: 478(7370):529-33; Wyce, A. et al., Oncotarget. 2013: 4(12):2419-29), sepsis (Nicodeme E. et al., Nature, 2010: 468(7327):1119-23), autoimmune and inflammatory diseases such as rheumatoid arthritis and multiple sclerosis (Mele D. A. et al., Journal of Experimental Medicine, 2013: 210(11):2181-90), heart failure (Anand P. et al., Cell, 2013: 154(3):569-82), and lung fibrosis (Tang X. et al., Molecular Pharmacology, 2013: 83(1):283-293).

There exists a need for chemical compounds which inhibit the activity of bromodomains, in particular compounds that inhibit the binding of BET proteins to acetylated lysine residues, and hence have utility in the treatment of, for example, autoimmune and inflammatory diseases, and cancers.

### SUMMARY OF THE INVENTION

In a first aspect, the present invention is directed to a compound of formula (I), or a salt thereof:

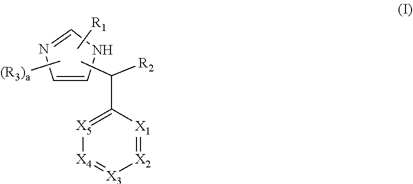

wherein
R₁ represents

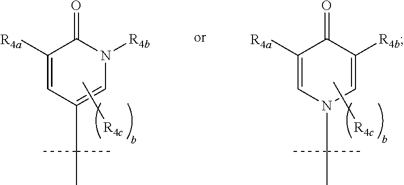

R₂ is hydrogen or C₁₋₃alkyl;
each R₃ is independently selected from the group consisting of halogen, —CN, —C₁₋₃alkyl, C₁₋₃alkoxy, —NO₂, —CONR₅R₆, —NR₅COR₆, —NR₅CO₂R₆, —OCOR₆, —CO₂R₆, —SO₂NR₅R₆, —NR₅SO₂R₆, —SO₂R₆, —R₆, —NR₅R₆, and —OR₆, with the proviso that when a is 2, one R₃ is selected from the group consisting of halogen, —CN, —C₁₋₃alkyl and C₁₋₃alkoxy;
R₄ₐ is hydrogen, C₁₋₃alkyl, C₁₋₃alkoxy, halogen, —CN, —OH, or —NR₇R₈;
R_{4b} is hydrogen or C₁₋₃alkyl;
each R_{4c} is independently selected from the group consisting of C₁₋₃alkyl, C₁₋₃alkoxy, halogen, —CN, —OH, and —NR₇R₈;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl and ═O;
Z is hydrogen, C₁₋₃alkyl, cycloalkyl, heterocycloalkyl, aryl, heteroaryl, —SO₂NR₉R₁₀, —NR₉SO₂R₁₀, —SO₂R₉, or —NR₉R₁₀, wherein the C₁₋₃alkyl, cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
X₁, X₂, X₃, X₄ and X₅ are each independently selected from CR₁₁ and N with the proviso that zero, one or two of X₁, X₂, X₃, X₄ and X₅ are N;
each R₇ is independently selected from hydrogen or CH₃;
each R₈ is independently selected from hydrogen or C₁₋₃alkyl;
R₉ is hydrogen or C₁₋₃alkyl;
R₁₀ is hydrogen or C₁₋₃alkyl;
or when Z is —SO₂NR₉R₁₀, R₉ and R₁₀ together with the nitrogen to which they are attached may form a heterocycloalkyl;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
or any two R₁₁ groups on adjacent carbon atoms together with the atoms to which they are attached may form a 5- or 6-membered heteroaryl group;
a is 0, 1 or 2;
b is 0, 1 or 2.

Compounds of the invention have been found to inhibit the binding of bromodomain containing proteins, in particular, the binding of the BET family of bromodomain containing proteins to, for example, acetylated lysine residues. Compounds of formula (I), or pharmaceutically acceptable salts thereof, may thus have use in therapy, for example in the treatment of autoimmune and inflammatory diseases, such as rheumatoid arthritis, and cancers.

The present invention is further directed to methods of treatment of autoimmune and inflammatory diseases and cancers through inhibition of the function of bromodomain containing proteins, for example members of the BET family of bromodomain containing proteins, which comprises administering to a subject in need thereof, a therapeutically effective amount of a compound of formula (I), or a pharmaceutically acceptable salt thereof.

In a further aspect, the present invention is directed to pharmaceutical compositions comprising a compound of formula (I), or a pharmaceutically acceptable salt thereof, and one or more pharmaceutically acceptable excipients.

### DETAILED DESCRIPTION OF THE INVENTION

### Definitions

As used herein, the term “bromodomain” refers to evolutionary and structurally conserved modules (approximately 110 amino acids in length) that bind acetylated lysine residues, such as those on the N-terminal tails of histones. They are protein domains that are found as part of much larger bromodomain containing proteins (BCPs), many of which have roles in regulating gene transcription and/or chromatin remodelling. The human genome encodes for at least 57 bromodomains.

As used herein, the term “BET” refers to the bromodomain and extraterminal domain family of bromodomain containing proteins which include BRD2, BRD3, BRD4 and BRDT.

As used herein, the term “BET inhibitor” refers to a compound that is capable of inhibiting the binding of one or more BET family bromodomain containing proteins (e.g. BRD2, BRD3, BRD4 or BRDT) to, for example, acetylated lysine residues.

As used herein, the term “alkyl” refers to a saturated hydrocarbon chain, straight or branched, having the specified number of carbon atoms. For example, C₁₋₆ alkyl refers to an alkyl group having from 1 to 6 carbon atoms. Unless otherwise stated, alkyl groups are unsubstituted. The term “alkyl” includes, but is not limited to, methyl, ethyl, propyl (n-propyl and isopropyl), butyl (n-butyl, sec-butyl, isobutyl and tert-butyl), pentyl, and hexyl.

As used herein, the term “alkylene” refers to a divalent radical derived from a straight or branched, saturated hydrocarbon chain of, for example, 1 to 3 carbon atoms (C₁₋₃alkylene). Examples of alkylene include —CH₂—, —CH₂CH₂—, and —CH₂CH₂CH₂—.

As used herein, the term “alkoxy” refers to an —O-alkyl group wherein “alkyl” is defined above.

As used herein, the term “cycloalkyl” refers to a saturated, monocyclic, hydrocarbon ring having 3 (cyclopropyl), 4 (cyclobutyl), 5 (cyclopentyl), 6 (cyclohexyl) or 7 (cycloheptyl) carbon atoms.

As used herein, the term “halogen” refers to fluoro, chloro, bromo and iodo.

As used herein, the term “heterocycloalkyl” refers to a saturated or unsaturated 3 to 7 membered monocyclic or bicyclic ring, which must contain 1 or 2 non-carbon atoms, which are selected from nitrogen, oxygen, and sulfur. Heterocycloalkyl groups may contain one or more C(O), S(O) or SO₂ groups. However, heterocycloalkyl groups are not aromatic. Heterocycloalkyl groups containing more than one heteroatom may contain different heteroatoms. “5 or 6 membered heterocycloalkyl” refers to a saturated or unsaturated 5 or 6 membered monocyclic ring, which must contain 1 or 2 non-carbon atoms, which are selected from nitrogen, oxygen, and sulfur. Heterocycloalkyl includes, but is not limited to, pyrrolidine, piperidine, piperazine, oxetane, tetrahydrofuran, tetrahydro-2H-pyran, morpholine, morpholine-3-one, piperidin-2-one, pyrimidine-2,4(1H,3H)-dione, thiomorpholine, and thiomorpholine 1,1-dioxide.

As used herein, the term “aryl” refers to a monocyclic or bicyclic, hydrocarbon, aromatic radical. Aryl includes, for example, phenyl and naphthyl.

As used herein, the term “heteroaryl” refers to a monocyclic or bicyclic, aromatic radical containing one or more heteroatoms selected from S, N and O. Illustrative examples of heteroaryl useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 1,3-benzodioxolyl, benzothienyl, indolizinyl, indolyl, isoindolyl, benzimidazolyl, benzoxazolyl, benzthiazolyl, benzoisothiazolyl, indazolyl, imidazopyridinyl, pyrazolopyridinyl, benzotriazolyl, triazolopyridinyl, purinyl, quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the phrase “optionally substituted” indicates that a group may be unsubstituted or substituted with one or more substituents as defined herein. “Substituted” in reference to a group indicates that a hydrogen atom attached to a member atom within a group is replaced by one of the defined substituents.

As used herein, the term “pharmaceutically acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively. Furthermore, pharmaceutically-acceptable salts of the compound of formula (I) may be prepared during further processing of the free acid or base form, for example in situ during manufacture into a pharmaceutical formulation.

As used herein, the term “treatment” refers to prophylaxis of the condition, ameliorating or stabilising the specified condition, reducing or eliminating the symptoms of the condition, slowing or eliminating the progression of the condition, and preventing or delaying reoccurrence of the condition in a previously afflicted patient or subject. In one embodiment, treatment refers to ameliorating or stabilising a specified condition, reducing or eliminating the symptoms of the condition, or slowing or eliminating the progression of the condition.

As used herein, the term “therapeutically effective amount” refers to the quantity of a compound of formula (I), or a pharmaceutically acceptable salt thereof, which will elicit the desired biological response in an animal or human body.

As used herein, the term “subject” refers to an animal or human body.

It is to be understood that references herein to “compound(s) of the invention” mean a compound of formula (I) as the free base, or as a salt, for example a pharmaceutically acceptable salt.

### STATEMENT OF THE INVENTION

In a first aspect, the present invention provides a compound of formula (I), or a salt thereof:

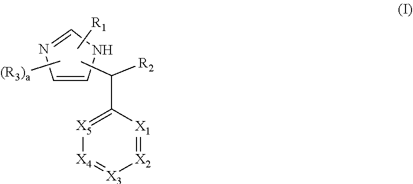

wherein
R₁ represents

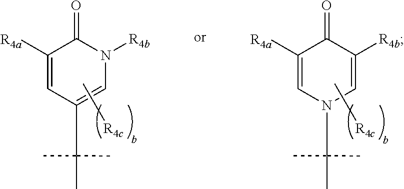

R₂ is hydrogen or C₁₋₃alkyl;
each R₃ is independently selected from the group consisting of halogen, —CN, —C₁₋₃alkyl, C₁₋₃alkoxy, —NO₂, —CONR₅R₆, —NR₅COR₆, —NR₅CO₂R₆, —OCOR₆, —CO₂R₆, —SO₂NR₅R₆, —NR₅SO₂R₆, —SO₂R₆, —R₆, —NR₅R₆, and —OR₆, with the proviso that when a is 2, one R₃ is selected from the group consisting of halogen, —CN, —C₁₋₃alkyl and C₁₋₃alkoxy;
R₄ₐ is hydrogen, C₁₋₃alkyl, C₁₋₃alkoxy, halogen, —CN, —OH, or —NR₇R₈;
R_{4b} is hydrogen or C₁₋₃alkyl;
each R_{4c} is independently selected from the group consisting of C₁₋₃alkyl, C₁₋₃alkoxy, halogen, —CN, —OH, and —NR₇R₈;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl and ═O;
Z is hydrogen, C₁₋₃alkyl, cycloalkyl, heterocycloalkyl, aryl, heteroaryl, —SO₂NR₉R₁₀, —NR₉SO₂R₁₀, —SO₂R₉, or —NR₉R₁₀, wherein the C₁₋₃alkyl, cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
X₁, X₂, X₃, X₄ and X₅ are each independently selected from CR₁₁ and N with the proviso that zero, one or two of X₁, X₂, X₃, X₄ and X₅ are N;
each R₇ is independently selected from hydrogen or CH₃;
each R₈ is independently selected from hydrogen or C₁₋₃alkyl;
R₉ is hydrogen or C₁₋₃alkyl;
R₁₀ is hydrogen or C₁₋₃alkyl;
or when Z is —SO₂NR₉R₁₀, R₉ and R₁₀ together with the nitrogen to which they are attached may form a heterocycloalkyl;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
or any two R₁₁ groups on adjacent carbon atoms together with the atoms to which they are attached may form a 5- or 6-membered heteroaryl group;
a is 0, 1 or 2;
b is 0, 1 or 2.

In one embodiment, the present invention provides a compound of formula (Ia-Ie), or a salt thereof:

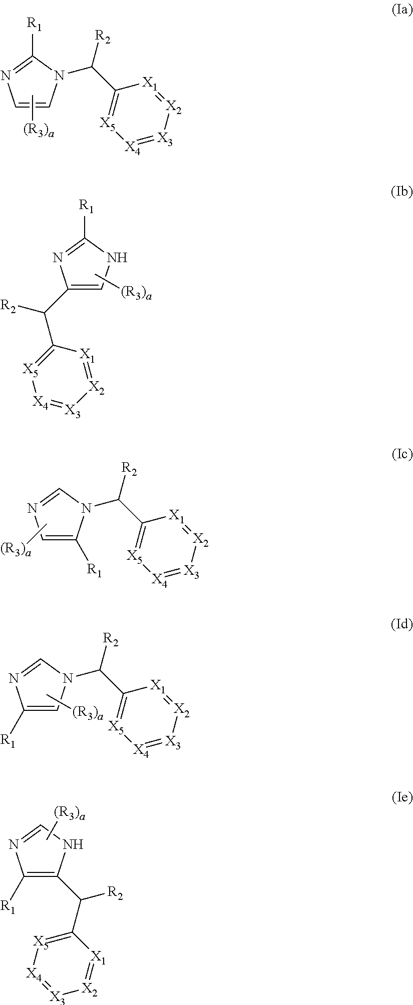

In a further embodiment, the present invention provides a compound of formula (Ia), (Ic) or (Ie), or a salt thereof:

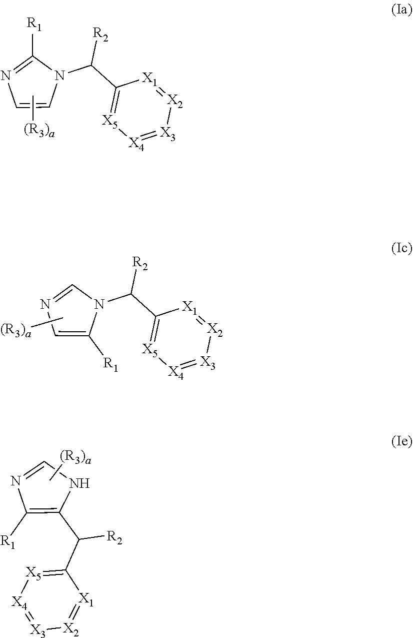

In a further embodiment, the present invention provides a compound of formula (Ia), or a salt thereof:

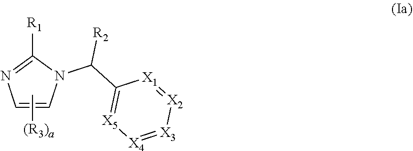

wherein R₁, R₂, R₃, a, X₁, X₂, X₃, X₄ and X₅ are as defined in claim 1.

In a further embodiment, each R₃ is independently selected from the group consisting of halogen, —CN, —C₁₋₃alkyl, C₁₋₃alkoxy, —NO₂, —CONR₅R₆, —NR₅COR₆, —OCOR₆, —CO₂R₆, —SO₂NR₅R₆, —NR₅SO₂R₆, —SO₂R₆, —R₆, —NR₅R₆, and —OR₆, with the proviso that when a is 2, one R₃ is selected from the group consisting of halogen, —CN, —C₁₋₃alkyl and C₁₋₃alkoxy.

In a further embodiment, the present invention is directed to a compound of formula (I), or salts thereof:

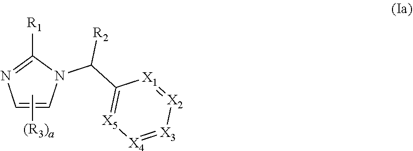

wherein,
R₁ represents

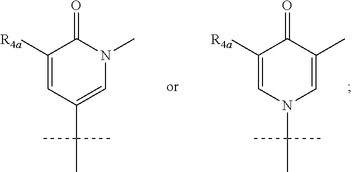

R₂ is hydrogen or C₁₋₃alkyl;
each R₃ is independently selected from the group consisting of halogen, —CN, —C₁₋₃alkyl, C₁₋₃alkoxy, —NO₂, —CONR₅R₆, —NR₅COR₆, —OCOR₆, —CO₂R₆, —SO₂NR₅R₆, —NR₅SO₂R₆, —SO₂R₆, —R₆, —NR₅R₆, and —OR₆, with the proviso that when a is 2, one R₃ is selected from the group consisting of halogen, —CN, —C₁₋₃alkyl and C₁₋₃alkoxy;
R₄ₐ is hydrogen, CH₃ or —OCH₃;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl and ═O;
Z is hydrogen, C₁₋₃alkyl, cycloalkyl, heterocycloalkyl, aryl, heteroaryl, —SO₂NR₉R₁₀, —NR₉SO₂R₁₀, —SO₂R₉, or —NR₉R₁₀, wherein the C₁₋₃alkyl, cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
X₁, X₂, X₃, X₄ and X₅ are each independently selected from CR₁₁ and N with the proviso that zero, one or two of X₁, X₂, X₃, X₄ and X₅ are N;
R₉ is hydrogen or C₁₋₃alkyl;
R₁₀ is hydrogen or C₁₋₃alkyl;
or when Z is —SO₂NR₉R₁₀, R₉ and R₁₀ together with the nitrogen to which they are attached may form a heterocycloalkyl;
each R₁₁ is independently selected from hydrogen, C₁₋₃alkyl, C₁₋₃alkoxy, halogen, —CN, —OH, —OCF₃, —OCHF₂, —CH₂OH, —CH₂CH₂OH, and CF₃;
or any two R₁₁ groups on adjacent carbon atoms together with the atoms to which they are attached may form a 5- or 6-membered heteroaryl group;
a is 0, 1 or 2.

In a further embodiment, R₁ represents

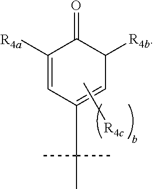

In a further embodiment, R₁ represents

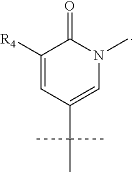

In a further embodiment, R₄ₐ is hydrogen, CH₃ or —OCH₃.

In a further embodiment, R₄ₐ is CH₃.

In a further embodiment, R₄ₐ is —OCH₃.

In a further embodiment, R_{4b} is C₁₋₃alkyl.

In a further embodiment, R_{4b} is CH₃.

In a further embodiment, b is 0.

In a further embodiment, R₄ₐ is hydrogen, CH₃ or —OCH₃, R_{4b} is CH₃ and b is 0.

In a further embodiment, R₂ is hydrogen or CH₃.

In a further embodiment, R₂ is hydrogen.

In a further embodiment, a is 1.

In a further embodiment, a is 2.

In a further embodiment, each R₃ is independently selected from the group consisting of halogen, —CN, —C₁₋₃alkyl, and C₁₋₃alkoxy.

In a further embodiment, each R₃ independently represents chloro or bromo.

In a further embodiment, a is 1 and R₃ is —CONR₅R₆ or —NR₅COR₆.

In a further embodiment, R₅ is hydrogen.

In a further embodiment, R₆ is —Y—Z, wherein Y is a bond or C₁₋₃alkylene and Z is selected from the group consisting of hydrogen, C₁₋₃alkyl, cycloalkyl, heterocycloalkyl, aryl and heteroaryl.

In a further embodiment, Z is a 5- or 6-membered heterocycloalkyl containing 1 or 2 non-carbon atoms independently selected from nitrogen and oxygen.

In a further embodiment, Z is pyrrolidinyl, piperidinyl, piperazinyl, tetrahydrofuranyl, tetrahydro-2H-pyranyl, or morpholinyl.

In a further embodiment, Z is phenyl or a 5- or 6-membered heteroaryl.

In a further embodiment, X₁, X₂, X₃, X₄ and X₅ each represent CR₁₁.

In a further embodiment, one or two of X₁, X₂, X₃, X₄ and X₅ represent N.

In a further embodiment, one of X₁, X₂, X₃, X₄ and X₅ represent N.

In a further embodiment, X₁ and X₅, or X₂ and X₄ represent N.

In a further embodiment, each R₁₁ is independently selected from hydrogen and C₁₋₃alkyl.

In a further embodiment, one or two R₁₁ groups are independently selected from C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃, and the remaining R₁₁ groups represent hydrogen.

In a further embodiment, the present invention provides compounds of formula (I), or salts thereof:

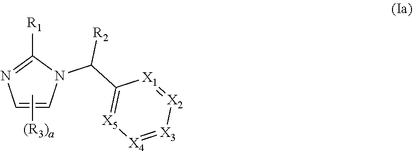

wherein,
R₁ represents

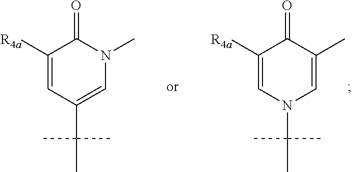

R₂ is hydrogen or C₁₋₃alkyl;
each R₃ is independently selected from the group consisting of halogen, —CN, —C₁₋₃alkyl, C₁₋₃alkoxy, —NO₂, —CONR₅R₆, —NR₅COR₆, —OCOR₆, —CO₂R₆, —SO₂NR₅R₆, —NR₅SO₂R₆, —SO₂R₆, —R₆, —NR₅R₆, and —OR₆, with the proviso that when a is 2, one R₃ is selected from the group consisting of halogen, —CN, —C₁₋₃alkyl and C₁₋₃alkoxy;
R₄ₐ is hydrogen, CH₃ or —OCH₃;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl and ═O;
Z is phenyl or a 5- or 6-membered heterocycloalkyl containing 1 or 2 non-carbon atoms independently selected from nitrogen and oxygen, wherein Z can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH; X₁, X₂, X₃, X₄ and X₅ are each independently selected from CR₁₁ and N with the proviso that zero, one or two of X₁, X₂, X₃, X₄ and X₅ are N;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

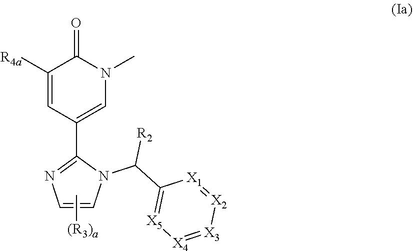

wherein:
R₂ is hydrogen;
each R₃ is independently selected from the group consisting of halogen, —CN, —C₁₋₃alkyl, C₁₋₃alkoxy, —NO₂, —CONR₅R₆, —NR₅COR₆, —OCOR₆, —CO₂R₆, —SO₂NR₅R₆, —NR₅SO₂R₆, —SO₂R₆, —R₆, —NR₅R₆, and —OR₆, with the proviso that when a is 2, one R₃ is selected from the group consisting of halogen, —CN, —C₁₋₃alkyl and C₁₋₃alkoxy;
R₄ₐ is hydrogen, CH₃ or —OCH₃;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is phenyl or a 5- or 6-membered heterocycloalkyl containing 1 or 2 non-carbon atoms independently selected from nitrogen and oxygen, wherein Z can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
X₁, X₂, X₃, X₄ and X₅ each represent CR₁₁;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

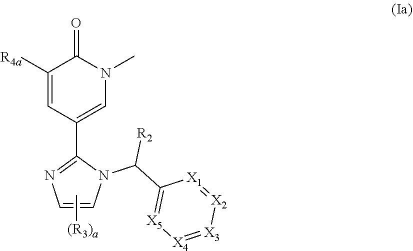

wherein,
R₂ is hydrogen or C₁₋₃alkyl;
each R₃ is independently selected from the group consisting of halogen, —CN, —C₁₋₃alkyl and C₁₋₃alkoxy;
R₄ₐ is CH₃ or —OCH₃;
X₁, X₂, X₃, X₄ and X₅ are each independently selected from CR₁₁ and N with the proviso that zero, one or two of X₁, X₂, X₃, X₄ and X₅ are N;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

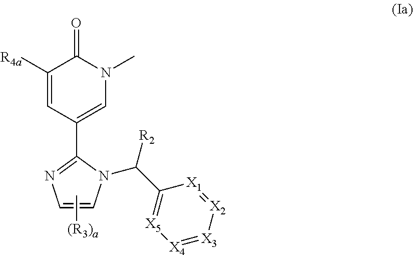

wherein,
R₂ is hydrogen or C₁₋₃alkyl;
each R₃ is independently selected from the group consisting of halogen, —CN, —C₁₋₃alkyl and C₁₋₃alkoxy;
R₄ₐ is CH₃ or —OCH₃;
one or two of X₁, X₂, X₃, X₄ and X₅ represent N and the others represent CR₁₁;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

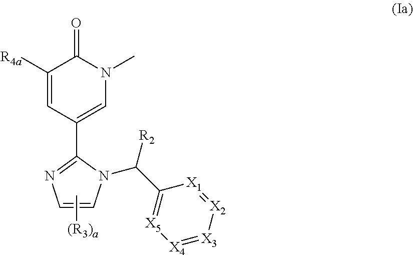

wherein,
R₂ is hydrogen or C₁₋₃alkyl;
each R₃ is independently selected and represents halogen selected from chloro, fluoro and bromo;
R₄ₐ is CH₃ or —OCH₃;
one or two of X₁, X₂, X₃, X₄ and X₅ represent N and the others represent CR₁₁;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

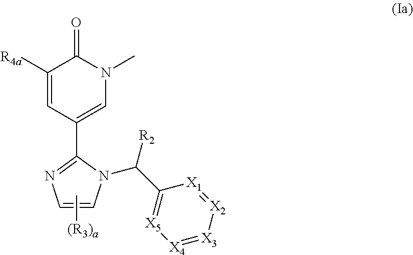

wherein,
R₂ is hydrogen or C₁₋₃alkyl;
each R₃ is independently selected from the group consisting of halogen, —CN, —C₁₋₃alkyl, C₁₋₃alkoxy, —CONR₅R₆, —NR₅COR₆, —OCOR₆, —CO₂R₆, —NR₅R₆, with the proviso that when a is 2, one R₃ is selected from the group consisting of halogen, —CN, —C₁₋₃alkyl and C₁₋₃alkoxy;
R₄ₐ is CH₃ or —OCH₃;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is hydrogen, C₁₋₃alkyl, cycloalkyl, heterocycloalkyl, aryl, heteroaryl, wherein C₁₋₃alkyl, cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
X₁, X₂, X₃, X₄ and X₅ are each independently selected from CR₁₁ and N with the proviso that zero, one or two of X₁, X₂, X₃, X₄ and X₅ are N;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

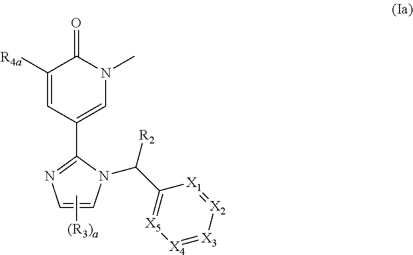

wherein,
R₂ is hydrogen or C₁₋₃alkyl;
each R₃ is independently selected from the group consisting of halogen, —CN, —C₁₋₃alkyl, C₁₋₃alkoxy, —CONR₅R₆, —NR₅COR₆, —OCOR₆, —CO₂R₆, —NR₅R₆, with the proviso that when a is 2,
one R₃ is selected from the group consisting of hydrogen, halogen, —CN, —C₁₋₃alkyl and C₁₋₃alkoxy;
R₄ₐ is CH₃ or —OCH₃;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is hydrogen, C₁₋₃alkyl, cycloalkyl, heterocycloalkyl, aryl, heteroaryl, wherein C₁₋₃alkyl, cycloalkyl, heterocycloalkyl, aryl or heteroaryl can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
one or two of X₁, X₂, X₃, X₄ and X₅ represent N and the others represent CR₁₁;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

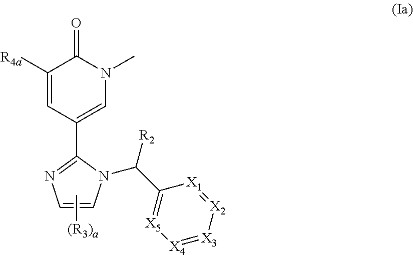

wherein,
R₂ is hydrogen or C₁₋₃alkyl;
each R₃ is independently selected from the group consisting of halogen, —CN, —C₁₋₃alkyl, C₁₋₃alkoxy, —CONR₅R₆, —NR₅COR₆, —OCOR₆, —CO₂R₆, —NR₅R₆, with the proviso that when a is 2, one R₃ is selected from the group consisting of halogen, —CN, —C₁₋₃alkyl and C₁₋₃alkoxy;
R₄ₐ is CH₃ or —OCH₃;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is phenyl or a 5- or 6-membered heterocycloalkyl containing 1 or 2 non-carbon atoms independently selected from nitrogen and oxygen, wherein Z can be optionally substituted with one or two groups independently selected from C₁₋₃alkyl, C₁₋₃alkoxy, halogen, —NH₂, —CH₂NH₂, —CO₂H, —OH, —CN, and —CH₂OH;
X₁, X₂, X₃, X₄ and X₅ are each independently selected from CR₁₁ and N with the proviso that zero, one or two of X₁, X₂, X₃, X₄ and X₅ are N;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

In a further embodiment, the present invention provides compounds of formula (Ia), or salts thereof:

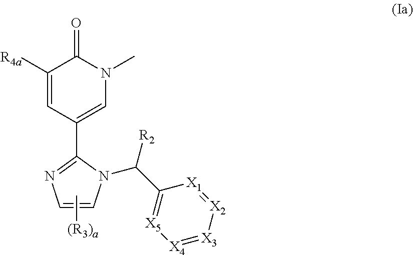

wherein,
R₂ is hydrogen or C₁₋₃alkyl;
each R₃ is independently selected from the group consisting of halogen, —CN, —C₁₋₃alkyl, C₁₋₃alkoxy, —CONR₅R₆, —NR₅COR₆, —OCOR₆, —CO₂R₆, —NR₅R₆, with the proviso that when a is 2, one R₃ is selected from the group consisting of halogen, —CN, —C₁₋₃alkyl and C₁₋₃alkoxy;
R₄ₐ is CH₃ or —OCH₃;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl;
Z is phenyl or a 5- or 6-membered heterocycloalkyl containing 1 or 2 non-carbon atoms independently selected from nitrogen and oxygen, wherein Z can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
one or two of X₁, X₂, X₃, X₄ and X₅ represent N and the others represent CR₁₁;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃ with the proviso that no more than two R₁₁ groups represent C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
a is 0, 1 or 2.

Specific examples of compounds of formula (I) are:
5-(1-benzyl-1H-imidazol-2-yl)-1,3-dimethylpyridin-2(1H)-one;
1,3-dimethyl-5-(1-(pyridin-3-ylmethyl)-1H-imidazol-2-yl)pyridin-2(1H)-one;
1,3-dimethyl-5-(1-(pyridin-4-ylmethyl)-1H-imidazol-2-yl)pyridin-2(1H)-one;
1,3-dimethyl-5-(1-(pyridin-2-ylmethyl)-1H-imidazol-2-yl)pyridin-2(1H)-one;
1,3-dimethyl-5-(1-(quinolin-8-ylmethyl)-1H-imidazol-2-yl)pyridin-2(1H)-one;
4-((2-(1,5-dimethyl-6-oxo-1,6-dihydropyridin-3-yl)-1H-imidazol-1-yl)methyl)benzonitrile;
5-((2-(1,5-dimethyl-6-oxo-1,6-dihydropyridin-3-yl)-1H-imidazol-1-yl)methyl)picolinonitrile;
5-(1-(3-methoxybenzyl)-1H-imidazol-2-yl)-1,3-dimethylpyridin-2(1H)-one;
1,3-dimethyl-5-(1-(4-(methylsulfonyl)benzyl)-1H-imidazol-2-yl)pyridin-2(1H)-one;
methyl 1-benzyl-2-(1,5-dimethyl-6-oxo-1,6-dihydropyridin-3-yl)-1H-imidazole-4-carboxylate;
methyl 1-benzyl-2-(1,5-dimethyl-6-oxo-1,6-dihydropyridin-3-yl)-1H-imidazole-5-carboxylate;
5-(1-benzyl-4-chloro-1H-imidazol-2-yl)-1,3-dimethylpyridin-2(1H)-one;
1,3-dimethyl-5-(1-(1-phenylethyl)-1H-imidazol-2-yl)pyridin-2(1H)-one;
1,3-dimethyl-5-(1-(pyrimidin-2-ylmethyl)-1H-imidazol-2-yl)pyridin-2(1H)-one;
1,3-dimethyl-5-(1-(pyrimidin-5-ylmethyl)-1H-imidazol-2-yl)pyridin-2(1H)-one;
1,3-dimethyl-5-(1-(3-(trifluoromethyl)benzyl)-1H-imidazol-2-yl)pyridin-2(1H)-one;
5-(1-(2-chlorobenzyl)-1H-imidazol-2-yl)-1,3-dimethylpyridin-2(1H)-one;
1,3-dimethyl-5-(1-(4-methylbenzyl)-1H-imidazol-2-yl)pyridin-2(1H)-one;
5-(1-(3,4-dichlorobenzyl)-1H-imidazol-2-yl)-1,3-dimethylpyridin-2(1H)-one;
5-(1-(4-methoxybenzyl)-1H-imidazol-2-yl)-1,3-dimethylpyridin-2(1H)-one;
5-(1-(3,5-difluorobenzyl)-1H-imidazol-2-yl)-1,3-dimethylpyridin-2(1H)-one;
1,3-dimethyl-5-(1-(4-(trifluoromethyl)benzyl)-1H-imidazol-2-yl)pyridin-2(1H)-one;
5-(1-(2-methoxybenzyl)-1H-imidazol-2-yl)-1,3-dimethylpyridin-2(1H)-one;
5-(1-(3-chloro-5-fluoro-4-methoxybenzyl)-1H-imidazol-2-yl)-1,3-dimethylpyridin-2(1H)-one;
5-(1-(3-fluoro-4-methoxybenzyl)-1H-imidazol-2-yl)-1,3-dimethylpyridin-2(1H)-one;
(S)-5-(1-benzyl-4-(2-(hydroxymethyl)pyrrolidine-1-carbonyl)-1H-imidazol-2-yl)-1,3-dimethylpyridin-2(1H)-one;
1,3-dimethyl-5-(1-((6-methylpyridin-2-yl)methyl)-1H-imidazol-2-yl)pyridin-2(1H)-one;
5-(1-(2-fluorobenzyl)-1H-imidazol-2-yl)-1,3-dimethylpyridin-2(1H)-one;
5-(1-(4-fluorobenzyl)-1H-imidazol-2-yl)-1,3-dimethylpyridin-2(1H)-one;
1,3-dimethyl-5-(1-(4-(trifluoromethoxy)benzyl)-1H-imidazol-2-yl)pyridin-2(1H)-one;
5-(1-(4-(difluoromethoxy)benzyl)-1H-imidazol-2-yl)-1,3-dimethylpyridin-2(1H)-one;
5-(1-(2,3-dichlorobenzyl)-1H-imidazol-2-yl)-1,3-dimethylpyridin-2(1H)-one;
5-(1-(3-chlorobenzyl)-1H-imidazol-2-yl)-1,3-dimethylpyridin-2(1H)-one;
1-benzyl-2-(1,5-dimethyl-6-oxo-1,6-dihydropyridin-3-yl)-1H-imidazole-4-carboxylic acid;
1,3-dimethyl-5-(1-((5-methylpyridin-3-yl)methyl)-1H-imidazol-2-yl)pyridin-2(1H)-one;
1,3-dimethyl-5-(1-(3-methylbenzyl)-1H-imidazol-2-yl)pyridin-2(1H)-one;
5-(1-(3-fluorobenzyl)-1H-imidazol-2-yl)-1,3-dimethylpyridin-2(1H)-one;
5-(1-(4-chlorobenzyl)-1H-imidazol-2-yl)-1,3-dimethylpyridin-2(1H)-one;
1,3-dimethyl-5-(1-(pyridazin-3-ylmethyl)-1H-imidazol-2-yl)pyridin-2(1H)-one;
5-(1-benzyl-4-nitro-1H-imidazol-2-yl)-1,3-dimethylpyridin-2(1H)-one;
5-(5-chloro-1-(1-phenylethyl)-1H-imidazol-2-yl)-1,3-dimethylpyridin-2(1H)-one;
1-benzyl-2-(1,5-dimethyl-6-oxo-1,6-dihydropyridin-3-yl)-N,N-dimethyl-1H-imidazole-4-carboxamide;
5-(1-benzyl-4-(morpholine-4-carbonyl)-1H-imidazol-2-yl)-1,3-dimethylpyridin-2(1H)-one;
5-(1-benzyl-4-(pyrrolidine-1-carbonyl)-1H-imidazol-2-yl)-1,3-dimethylpyridin-2(1H)-one;
5-(4-(azetidine-1-carbonyl)-1-benzyl-1H-imidazol-2-yl)-1,3-dimethylpyridin-2(1H)-one;
1-benzyl-2-(1,5-dimethyl-6-oxo-1,6-dihydropyridin-3-yl)-N-methyl-1H-imidazole-4-carboxamide;
5-(1-benzyl-5-chloro-1H-imidazol-2-yl)-1,3-dimethylpyridin-2(1H)-one;
1-benzyl-2-(1,5-dimethyl-6-oxo-1,6-dihydropyridin-3-yl)-N-ethyl-1H-imidazole-4-carboxamide;
1-benzyl-2-(1,5-dimethyl-6-oxo-1,6-dihydropyridin-3-yl)-N-(tetrahydrofuran-3-yl)-1H-imidazole-4-carboxamide;
5-(1-benzyl-4-(piperidine-1-carbonyl)-1H-imidazol-2-yl)-1,3-dimethylpyridin-2(1H)-one;
1-benzyl-2-(1,5-dimethyl-6-oxo-1,6-dihydropyridin-3-yl)-N-(tetrahydro-2H-pyran-4-yl)-1H-imidazole-4-carboxamide;
1-benzyl-2-(1,5-dimethyl-6-oxo-1,6-dihydropyridin-3-yl)-N-phenethyl-1H-imidazole-4-carboxamide;
N,1-dibenzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-1H-imidazole-4-carbonitrile;
1-benzyl-2-(1,5-dimethyl-6-oxo-1,6-dihydropyridin-3-yl)-1H-imidazole-5-carbonitrile;
5-(1-benzyl-4-bromo-5-chloro-1H-imidazol-2-yl)-1,3-dimethylpyridin-2(1H)-one;
N-(1-benzyl-2-(1,5-dimethyl-6-oxo-1,6-dihydropyridin-3-yl)-1H-imidazol-4-yl)acetamide;
5-(4-chloro-1-(pyridin-2-ylmethyl)-1H-imidazol-2-yl)-1,3-dimethylpyridin-2(1H)-one;
1-benzyl-2-(1,5-dimethyl-6-oxo-1,6-dihydropyridin-3-yl)-1H-imidazole-4-carboxamide;
5-(4-chloro-1-(1-(pyridin-2-yl)ethyl)-1H-imidazol-2-yl)-1,3-dimethylpyridin-2(1H)-one;
5-(5-chloro-1-(1-(pyridin-2-yl)ethyl)-1H-imidazol-2-yl)-1,3-dimethylpyridin-2(1H)-one;
5-(1-benzyl-4-fluoro-1H-imidazol-2-yl)-1,3-dimethylpyridin-2(1H)-one;
5-(1-benzyl-4-bromo-1H-imidazol-2-yl)-1,3-dimethylpyridin-2(1H)-one;
5-(1-benzyl-4-methyl-1H-imidazol-2-yl)-1,3-dimethylpyridin-2(1H)-one;
5-(4-chloro-1-((5-methoxypyridin-2-yl)methyl)-1H-imidazol-2-yl)-1,3-dimethylpyridin-2(1H)-one;
5-(4-chloro-1-((6-methoxypyridin-3-yl)methyl)-1H-imidazol-2-yl)-1,3-dimethylpyridin-2(1H)-one;
5-(1-benzyl-4-phenyl-1H-imidazol-2-yl)-1,3-dimethylpyridin-2(1H)-one;
1-benzyl-N-cyclopropyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-isopropyl-1H-imidazole-4-carboxamide;
1-benzyl-2-(1,5-dimethyl-6-oxo-1,6-dihydropyridin-3-yl)-N-((tetrahydrofuran-2-yl)methyl)-1H-imidazole-4-carboxamide;
5-(4-chloro-1-((5-methylpyridin-2-yl)methyl)-1H-imidazol-2-yl)-1,3-dimethylpyridin-2(1H)-one;
5-(4-chloro-1-((5-fluoropyridin-2-yl)methyl)-1H-imidazol-2-yl)-1,3-dimethylpyridin-2(1H)-one;
5-((4-chloro-2-(1,5-dimethyl-6-oxo-1,6-dihydropyridin-3-yl)-1H-imidazol-1-yl)methyl)picolinonitrile;
1-benzyl-2-(1,5-dimethyl-6-oxo-1,6-dihydropyridin-3-yl)-N-(2-(dimethylamino)ethyl)-1H-imidazole-4-carboxamide;
1-benzyl-2-(1,5-dimethyl-6-oxo-1,6-dihydropyridin-3-yl)-N-(2-(N-methylsulfamoyl)ethyl)-1H-imidazole-4-carboxamide;
1-benzyl-2-(1,5-dimethyl-6-oxo-1,6-dihydropyridin-3-yl)-N-(2-(pyrrolidin-1-ylsulfonyl)ethyl)-1H-imidazole-4-carboxamide;
1-benzyl-2-(1,5-dimethyl-6-oxo-1,6-dihydropyridin-3-yl)-N-(2-sulfamoylethyl)-1H-imidazole-4-carboxamide;
1-benzyl-2-(1,5-dimethyl-6-oxo-1,6-dihydropyridin-3-yl)-N-(2-(piperazin-1-yl)ethyl)-1H-imidazole-4-carboxamide;
1-benzyl-2-(1,5-dimethyl-6-oxo-1,6-dihydropyridin-3-yl)-N-(piperidin-4-ylmethyl)-1H-imidazole-4-carboxamide;
1-benzyl-2-(1,5-dimethyl-6-oxo-1,6-dihydropyridin-3-yl)-N-(pyrrolidin-3-ylmethyl)-1H-imidazole-4-carboxamide;
1-benzyl-2-(1,5-dimethyl-6-oxo-1,6-dihydropyridin-3-yl)-N-((tetrahydrofuran-3-yl)methyl)-1H-imidazole-4-carboxamide;
N-((4-(aminomethyl)cyclohexyl)methyl)-1-benzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-(2-morpholinoethyl)-1H-imidazole-4-carboxamide;
1-benzyl-2-(1,5-dimethyl-6-oxo-1,6-dihydropyridin-3-yl)-N-((1-isopropylpyrrolidin-3-yl)methyl)-1H-imidazole-4-carboxamide;
1-benzyl-2-(1,5-dimethyl-6-oxo-1,6-dihydropyridin-3-yl)-N-(morpholin-2-ylmethyl)-1H-imidazole-4-carboxamide;
1-benzyl-2-(1,5-dimethyl-6-oxo-1,6-dihydropyridin-3-yl)-N-((tetrahydro-2H-pyran-4-yl)methyl)-1H-imidazole-4-carboxamide;
1-benzyl-2-(1,5-dimethyl-6-oxo-1,6-dihydropyridin-3-yl)-N-((1-methylpyrrolidin-3-yl)methyl)-1H-imidazole-4-carboxamide;
1-benzyl-2-(1,5-dimethyl-6-oxo-1,6-dihydropyridin-3-yl)-N-((tetrahydro-2H-pyran-3-yl)methyl)-1H-imidazole-4-carboxamide;
1-benzyl-2-(1,5-dimethyl-6-oxo-1,6-dihydropyridin-3-yl)-N-((1-methylpiperidin-4-yl)methyl)-1H-imidazole-4-carboxamide;
N-(2-(4-aminopiperidin-1-yl)ethyl)-1-benzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-(2-(4-methylpiperazin-1-yl)ethyl)-1H-imidazole-4-carboxamide;
N-(((1r,4r)-4-aminocyclohexyl)methyl)-1-benzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-(2-(piperidin-1-yl)propyl)-1H-imidazole-4-carboxamide;
1-benzyl-2-(1,5-dimethyl-6-oxo-1,6-dihydropyridin-3-yl)-N-(2-(4-hydroxpiperidin-1-yl)-2-oxoethyl)-1H-imidazole-4-carboxamide;
4-((1-benzyl-2-(1,5-dimethyl-6-oxo-1,6-dihydropyridin-3-yl)-1H-imidazole-4-carboxamido)methyl)cyclohexanecarboxylic acid;
5-(1-(3-(2-hydroxyethyl)benzyl)-1H-imidazol-2-yl)-1,3-dimethylpyridin-2(1H)-one;
5-(1-benzyl-1H-imidazol-5-yl)-1,3-dimethylpyridin-2(1H)-one;
5-(1-benzyl-4-chloro-1H-imidazol-5-yl)-1,3-dimethylpyridin-2(1H)-one;
1-(1-benzyl-1H-imidazol-2-yl)-3,5-dimethylpyridin-4(1H)-one; and
tert-butyl (1-benzyl-2-(1,5-dimethyl-6-oxo-1,6-dihydropyridin-3-yl)-1H-imidazol-4-yl)carbamate, or salts thereof.

In a further embodiment, the present invention provides a compound, or a salt thereof, which is selected form the group consisting of:
5-(1-benzyl-4-bromo-5-chloro-1H-imidazol-2-yl)-1,3-dimethylpyridin-2(1H)-one;
5-(1-benzyl-4-chloro-1H-imidazol-2-yl)-1,3-dimethylpyridin-2(1H)-one;
5-(1-benzyl-4-bromo-1H-imidazol-2-yl)-1,3-dimethylpyridin-2(1H)-one;
5-(1-benzyl-4-phenyl-1H-imidazol-2-yl)-1,3-dimethylpyridin-2(1H)-one;
Methyl 1-benzyl-2-(1,5-dimethyl-6-oxo-1,6-dihydropyridin-3-yl)-1H-imidazole-5-carboxylate;
5-(5-chloro-1-(1-phenylethyl)-1H-imidazol-2-yl)-1,3-dimethylpyridin-2(1H)-one;
1-benzyl-2-(1,5-dimethyl-6-oxo-1,6-dihydropyridin-3-yl)-1H-imidazole-4-carbonitrile;
5-(1-benzyl-4-methyl-1H-imidazol-2-yl)-1,3-dimethylpyridin-2(1H)-one;
Methyl 1-benzyl-2-(1,5-dimethyl-6-oxo-1,6-dihydropyridin-3-yl)-1H-imidazole-4-carboxylate;
5-(1-(4-chlorobenzyl)-1H-imidazol-2-yl)-1,3-dimethylpyridin-2(1H)-one;
1-benzyl-2-(1,5-dimethyl-6-oxo-1,6-dihydropyridin-3-yl)-N-phenethyl-1H-imidazole-4-carboxamide;
N,1-dibenzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-1H-imidazole-4-carboxamide;
5-(4-chloro-1-(1-(pyridin-2-yl)ethyl)-1H-imidazol-2-yl)-1,3-dimethylpyridin-2(1H)-one;
5-(1-benzyl-4-fluoro-1H-imidazol-2-yl)-1,3-dimethylpyridin-2(1H)-one;
5-(4-chloro-1-((5-methylpyridin-2-yl)methyl)-1H-imidazol-2-yl)-1,3-dimethylpyridin-2(1H)-one.

In a further embodiment of the present invention, a compound of formula (I) is in the form of a free base. In one embodiment, the compound of formula (I) in the form of a free base is any one of the compounds of Examples 1 to 100.

Salts of the compounds of formula (I) include pharmaceutically acceptable salts and salts which may not be pharmaceutically acceptable but may be useful in the preparation of compounds of formula (I) and pharmaceutically acceptable salts thereof.

In one embodiment of the present invention, a compound of formula (I) is in the form of a pharmaceutically acceptable salt. In one embodiment, the compound of any of Example 1 to 96 is in the form of a pharmaceutically acceptable salt.

Compounds of formula (I) may contain an acidic or basic functional group and, thus, the skilled artisan will appreciate that pharmaceutically acceptable salts of the compounds of formula (I) may be prepared. Pharmaceutically acceptable salts of compounds of the invention may possess, for example, improved stability, solubility, and/or crystallinity, facilitating development as a medicine.

Compounds of formula (I) may contain a basic functional group and may be capable of forming pharmaceutically acceptable acid addition salts by treatment with an suitable acid (inorganic or organic acid). Representative pharmaceutically acceptable acid addition salts include hydrochloride, hydrobromide, nitrate, sulfate, bisulfate, sulfamate, phosphate, acetate, hydroxyacetate, phenylacetate, propionate, butyrate, isobutyrate, valerate, maleate, hydroxymaleate, acrylate, fumarate, maleate, tartrate, citrate, salicylate, p-aminosalicyclate, glycollate, lactate, heptanoate, phthalate, oxalate, succinate, benzoate, o-acetoxybenzoate, chlorobenzoate, methyl benzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate. In another embodiment, the pharmaceutically acceptable salt is the 1,2-ethanedisulphonic acid (edisylate) salt.

Compounds of formula (I) may contain an acidic functional group and suitable pharmaceutically-acceptable salts include salts of such acidic functional groups. Representative salts include pharmaceutically acceptable metal salts such as sodium, potassium, lithium, calcium, magnesium, aluminum, and zinc salts; pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, TEA, ethylenediamine, ethanolamine, diethanolamine, and cyclohexylamine.

For a review on suitable salts see Berge et al., J. Pharm. Sci., 66:1-19 (1977). The invention includes within its scope all possible stoichiometric and non-stoichiometric forms of the salts of the compounds of formula (I).

Salts may be formed using techniques well-known in the art, for example by precipitation from solution followed by filtration, or by evaporation of the solvent.

It will be appreciated that many organic compounds can form complexes with solvents in which they are reacted or from which they are precipitated or crystallised. These complexes are known as “solvates”. For example, a complex with water is known as a “hydrate”. Solvents with high boiling points and/or solvents with a high propensity to form hydrogen bonds such as water, ethanol, iso-propyl alcohol, and N-methyl pyrrolidinone may be used to form solvates. Methods for the identification of solvates include, but are not limited to, NMR and microanalysis. Compounds of formula (I), or salts thereof, may exist in solvated and unsolvated form.

Certain of the compounds of the invention may exist in tautomeric forms. It will be understood that the present invention encompasses all of the tautomers of the compounds of the invention whether as individual tautomers or as mixtures thereof.

The compounds of the invention may be in crystalline or amorphous form. The most thermodynamically stable crystalline form of a compound of the invention is of particular interest.

Crystalline forms of compounds of the invention may be characterised and differentiated using a number of conventional analytical techniques, including, but not limited to, X-ray powder diffraction (XRPD), infrared spectroscopy (IR), Raman spectroscopy, differential scanning calorimetry (DSC), thermogravimetric analysis (TGA) and solid-state nuclear magnetic resonance (ssNMR).

The present invention also includes all suitable isotopic variations of a compound of formula (I) or a pharmaceutically acceptable salt thereof. An isotopic variation of a compound of formula (I), or a pharmaceutically acceptable salt thereof, is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, fluorine and chlorine such as ²H, ³H, ¹³C, ¹⁴C, ¹⁵N, ¹⁷O, ¹⁸O, ¹⁸F and ³⁶Cl, respectively. Certain isotopic variations of a compound of formula (I) or a salt or solvate thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of a compound of formula (I), or a pharmaceutically salt thereof, can generally be prepared by conventional procedures such as by the illustrative methods or by the preparations described in the Examples hereafter using appropriate isotopic variations of suitable reagents.

The compounds of formula (I) and pharmaceutically acceptable salts thereof may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of formula (I), or in any chemical structure illustrated herein, is not specified the structure is intended to encompass all individual stereoisomers and all mixtures thereof. Thus, compounds of formula (I) and pharmaceutically acceptable salts thereof containing one or more chiral center may be used as racemic mixtures, enantiomerically enriched mixtures, or as enantiomerically pure individual stereoisomers.

Individual stereoisomers of a compound of formula (I), or a pharmaceutically acceptable salt thereof, which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

In one embodiment, a compound of the invention is capable of inhibiting the binding of one or more of the four known BET family bromodomain containing proteins (e.g. BRD2, BRD3, BRD4 and BRDt) to, for example, an acetylated lysine residue. In a further embodiment, the compound of formula (I), or a pharmaceutically acceptable salt thereof, is capable of inhibiting the binding of BRD4 to its cognate acetylated lysine residue. The compounds of the invention may possess an improved profile over known BET inhibitors, for example, certain compounds may have one or more of the following properties:
(i) potent BET inhibitory activity;
(ii) selectivity over other known bromodomain containing proteins outside of the BET family of proteins;
(iii) selectivity for a particular BET family member over other BET family members;
(iv) selectivity for one Binding Domain (i.e. BD1 over BD2 or vice versa) for any given BET family member;
(v) improved developability (e.g. desirable solubility profile, pharmacokinetics and pharmacodynamics); or
(vi) a reduced side-effect profile.

### STATEMENT OF USE

Compounds of formula (I), or pharmaceutically acceptable salts thereof, are BET inhibitors and thus may have therapeutic utility in the treatment of a variety of diseases or conditions related to systemic or tissue inflammation, inflammatory responses to infection or hypoxia, cellular activation and proliferation, lipid metabolism, fibrosis and in the prevention and treatment of viral infections.

BET inhibitors may be useful in the treatment of a wide variety of acute or chronic autoimmune or inflammatory conditions such as rheumatoid arthritis, osteoarthritis, acute gout, psoriasis, psoriatic arthritis, spondyloarthritis, systemic lupus erythematosus, pulmonary arterial hypertension (PAH), multiple sclerosis, inflammatory bowel disease (Crohn's disease and ulcerative colitis), asthma, chronic obstructive airways disease, pneumonitis, myocarditis, pericarditis, myositis, eczema, dermatitis (including atopic dermatitis), alopecia, vitiligo, bullous skin diseases, nephritis, vasculitis, hypercholesterolemia, atherosclerosis, Alzheimer's disease, depression, Sjögren's syndrome, sialoadenitis, central retinal vein occlusion, branched retinal vein occlusion, Irvine-Gass syndrome (post cataract and post-surgical), retinitis pigmentosa, pars planitis, birdshot retinochoroidopathy, epiretinal membrane, cystic macular edema, parafoveal telengiectasis, tractional maculopathies, vitreomacular traction syndromes, retinal detachment, neuroretinitis, idiopathic macular edema, retinitis, dry eye (keratoconjunctivitis Sicca), vernal keratoconjunctivitis, atopic keratoconjunctivitis, uveitis (such as anterior uveitis, pan uveitis, posterior uveitis, uveitis-associated macular edema), scleritis, diabetic retinopathy, diabetic macular edema, age-related macular dystrophy, hepatitis, pancreatitis, primary biliary cirrhosis, sclerosing cholangitis, acute alcoholic hepatitis, chronic alcoholic hepatitis, alcoholic steato-hepatitis, non-alcoholic steato-hepatitis (NASH), cirrhosis, Childs-Pugh cirrhosis, autoimmune hepatitis, fulminant hepatitis, chronic viral hepatitis, alcoholic liver disease, systemic sclerosis, sarcoidosis, neurosarcoidosis, Addison's disease, hypophysitis, thyroiditis, type I diabetes, giant cell arteritis, nephritis including lupus nephritis, vasculitis with organ involvement such as glomerulonephritis, vasculitis including giant cell arteritis, Wegener's granulomatosis, Polyarteritis nodosa, Behcet's disease, Kawasaki disease, Takayasu's arteritis, pyoderma gangrenosum, vasculitis with organ involvement, chronic organ transplant rejection and acute rejection of transplanted organs. The use of BET inhibitors for the treatment of rheumatoid arthritis and NASH are of particular interest.

In one embodiment, the acute or chronic autoimmune or inflammatory condition is a disorder of lipid metabolism via the regulation of APO-A1 such as hypercholesterolemia, atherosclerosis and Alzheimer's disease.

In another embodiment, the acute or chronic autoimmune or inflammatory condition is a respiratory disorder such as asthma or chronic obstructive airways disease.

In another embodiment, the acute or chronic autoimmune or inflammatory condition is a systemic inflammatory disorder such as rheumatoid arthritis, osteoarthritis, acute gout, psoriasis, systemic lupus erythematosus, multiple sclerosis or inflammatory bowel disease (Crohn's disease and ulcerative colitis).

In another embodiment, the acute or chronic autoimmune or inflammatory condition is multiple sclerosis.

In a further embodiment, the acute or chronic autoimmune or inflammatory condition is type I diabetes.

BET inhibitors may be useful in the treatment of diseases or conditions which involve inflammatory responses to infections with bacteria, viruses, fungi, parasites or their toxins, such as sepsis, acute sepsis, sepsis syndrome, septic shock, endotoxaemia, systemic inflammatory response syndrome (SIRS), multi-organ dysfunction syndrome, toxic shock syndrome, acute lung injury, ARDS (adult respiratory distress syndrome), acute renal failure, fulminant hepatitis, burns, acute pancreatitis, post-surgical syndromes, sarcoidosis, Herxheimer reactions, encephalitis, myelitis, meningitis, malaria and SIRS associated with viral infections such as influenza, herpes zoster, herpes simplex and coronavirus. In one embodiment, the disease or condition which involves an inflammatory response to an infection with bacteria, a virus, fungi, a parasite or their toxins is acute sepsis.

BET inhibitors may be useful in the treatment of conditions associated with ischaemia-reperfusion injury such as myocardial infarction, cerebro-vascular ischaemia (stroke), acute coronary syndromes, renal reperfusion injury, organ transplantation, coronary artery bypass grafting, cardio-pulmonary bypass procedures, pulmonary, renal, hepatic, gastro-intestinal or peripheral limb embolism.

BET inhibitors may be useful in the treatment of fibrotic conditions such as idiopathic pulmonary fibrosis, renal fibrosis, liver fibrosis, post-operative stricture, keloid scar formation, scleroderma (including morphea), cardiac fibrosis and cystic fibrosis.

BET inhibitors may be useful in the treatment of viral infections such as herpes simplex infections and reactivations, cold sores, herpes zoster infections and reactivations, chickenpox, shingles, human papilloma virus (HPV), human immunodeficiency virus (HIV), cervical neoplasia, adenovirus infections, including acute respiratory disease, poxvirus infections such as cowpox and smallpox and African swine fever virus. In one embodiment, the viral infection is a HPV infection of skin or cervical epithelia. In another embodiment, the viral infection is a latent HIV infection.

BET inhibitors may be useful in the treatment of cancer, including hematological (such as leukaemia, lymphoma and multiple myeloma), epithelial including lung, breast and colon carcinomas, midline carcinomas, mesenchymal, hepatic, renal and neurological tumours.

BET inhibitors may be useful in the treatment of one or more cancers selected from brain cancer (gliomas), glioblastomas, Bannayan-Zonana syndrome, Cowden disease, Lhermitte-Duclos disease, breast cancer, inflammatory breast cancer, colorectal cancer, Wilm's tumor, Ewing's sarcoma, rhabdomyosarcoma, ependymoma, medulloblastoma, colon cancer, head and neck cancer, kidney cancer, lung cancer, liver cancer, melanoma, squamous cell carcinoma, ovarian cancer, pancreatic cancer, prostate cancer, sarcoma cancer, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukaemia, erythroleukemia, malignant lymphoma, Hodgkins lymphoma, non-Hodgkins lymphoma, lymphoblastic T-cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), NUT-midline carcinoma and testicular cancer.

In one embodiment, the cancer is a leukaemia, for example a leukaemia selected from acute monocytic leukemia, acute myelogenous leukemia, chronic myelogenous leukemia, chronic lymphocytic leukemia and mixed lineage leukaemia (MLL). In another embodiment, the cancer is NUT-midline carcinoma. In another embodiment, the cancer is multiple myeloma. In another embodiment, the cancer is a lung cancer such as small cell lung cancer (SCLC). In another embodiment, the cancer is a neuroblastoma. In another embodiment, the cancer is Burkitt's lymphoma. In another embodiment, the cancer is cervical cancer. In another embodiment, the cancer is esophageal cancer. In another embodiment, the cancer is ovarian cancer. In another embodiment, the cancer is breast cancer. In another embodiment, the cancer is colorectal cancer. In another embodiment, the cancer is prostate cancer. In another embodiment, the cancer is castration-resistant prostate cancer.

In one embodiment, the disease or condition for which a BET inhibitor is indicated is selected from diseases associated with systemic inflammatory response syndrome, such as sepsis, burns, pancreatitis, major trauma, haemorrhage and ischaemia. In this embodiment, the BET inhibitor would be administered at the point of diagnosis to reduce the incidence of SIRS, the onset of shock, multi-organ dysfunction syndrome, which includes the onset of acute lung injury, ARDS, acute renal, hepatic, cardiac or gastro-intestinal injury and mortality. In another embodiment, the BET inhibitor would be administered prior to surgical or other procedures associated with a high risk of sepsis, haemorrhage, extensive tissue damage, SIRS or MODS (multiple organ dysfunction syndrome). In a particular embodiment, the disease or condition for which a BET inhibitor is indicated is sepsis, sepsis syndrome, septic shock and endotoxaemia. In another embodiment, the BET inhibitor is indicated for the treatment of acute or chronic pancreatitis. In another embodiment, the BET inhibitor is indicated for the treatment of burns.

In a further aspect, the present invention also provides a compound of formula (I) or a pharmaceutically acceptable salt thereof for use in therapy.

In a further aspect, the present invention provides a compound of formula (I) or a pharmaceutically acceptable salt thereof for use in the treatment of diseases or conditions for which a bromodomain inhibitor, in particular a BET inhibitor, is indicated, including each and all of the above listed indications.

In a further aspect, the present invention also provides a compound of formula (I), or a pharmaceutically acceptable salt thereof, for use in the treatment of autoimmune and inflammatory diseases, and cancer.

In a further aspect, the present invention provides a compound of formula (I), or a pharmaceutically acceptable salt thereof, for use in the treatment of rheumatoid arthritis.

In a further aspect, the present invention is directed to a method of treatment of an autoimmune or inflammatory disease or cancer, which comprises administering to a subject in need thereof, a therapeutically effective amount of a compound of formula (I), or a pharmaceutically acceptable salt thereof.

In yet a further aspect, the present invention is directed to a method of treating rheumatoid arthritis, which comprises administering to a subject in need thereof, a therapeutically effective amount of a compound of formula (I), or a pharmaceutically acceptable salt thereof.

In a further aspect, the present invention is directed to the use of a compound of formula (I), or a pharmaceutically acceptable salt thereof, in the manufacture of a medicament for use in the treatment of an autoimmune or inflammatory disease, or cancer.

In a further aspect, the present invention is directed to the use of a compound of formula (I), or a pharmaceutically acceptable salt thereof, in the manufacture of a medicament for use in the treatment of rheumatoid arthritis.

### Pharmaceutical Compositions/Routes of Administration/Dosages

While it is possible that for use in therapy, a compound of formula (I) as well as pharmaceutically acceptable salts thereof may be administered as the raw chemical, it is common to present the active ingredient as a pharmaceutical composition.

In a further aspect, there is provided a pharmaceutical composition comprising a compound of formula (I), or a pharmaceutically acceptable salt thereof, and one or more pharmaceutically acceptable excipients. In a further aspect, there is provided a pharmaceutical composition comprising a compound of formula (I), or a pharmaceutically acceptable salt thereof, and a pharmaceutically acceptable excipient.

The excipient(s) must be pharmaceutically acceptable and be compatible with the other ingredients of the composition. In accordance with another aspect of the invention there is also provided a process for the preparation of a pharmaceutical composition including admixing a compound of formula (I), or a pharmaceutically acceptable salt thereof, with one or more pharmaceutically acceptable excipients. The pharmaceutical composition can be used in the treatment of any of the diseases described herein.

Since the compounds of formula (I) are intended for use in pharmaceutical compositions it will be readily understood that they are each preferably provided in substantially pure form, for example, at least 85% pure, especially at least 98% pure (% in a weight for weight basis).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day.

Pharmaceutical compositions may be adapted for administration by any appropriate route, for example by the oral (including buccal or sublingual), rectal, inhaled, intranasal, topical (including buccal, sublingual or transdermal), ocular (including topical, intraocular, subconjunctival, episcleral, sub-Tenon), vaginal or parenteral (including subcutaneous, intramuscular, intravenous or intradermal) route. Such compositions may be prepared by any method known in the art of pharmacy, for example by bringing into association the active ingredient with the excipient(s).

In one aspect, the pharmaceutical composition is adapted for oral administration.

Pharmaceutical compositions adapted for oral administration may be presented as discrete units such as tablets or capsules; powders or granules; solutions or suspensions in aqueous or non-aqueous liquids; edible foams or whips; or oil-in-water liquid emulsions or water-in-oil liquid emulsions.

Powders suitable for incorporating into tablets or capsules may be prepared by reducing the compound to a suitable fine size (e.g. by micronisation) and mixing with a similarly prepared pharmaceutical excipient such as an edible carbohydrate, for example, starch or mannitol. Flavoring, preservative, dispersing and coloring agents, for example, may also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like.

Disintegrators include starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of formula (I) and pharmaceutically acceptable salts thereof can also be combined with a free flowing inert excipient and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Compositions for oral administration may be designed to provide a modified release profile so as to sustain or otherwise control the release of the therapeutically active agent.

Where appropriate, dosage unit compositions for oral administration can be microencapsulated. The composition may be prepared to prolong or sustain the release as for example by coating or embedding particulate material in polymers, wax or the like.

Pharmaceutical compositions for nasal or inhaled administration may conveniently be formulated as aerosols, solutions, suspensions, gels or dry powders.

For pharmaceutical compositions suitable for and/or adapted for inhaled administration, it is preferred that a compound of formula (I) or a pharmaceutically acceptable salt thereof, is in a particle-size-reduced form e.g. obtained by micronisation. The preferable particle size of the size-reduced (e.g. micronised) compound or salt is defined by a D50 value of about 0.5 to about 10 microns (for example as measured using laser diffraction).

For pharmaceutical compositions suitable for and/or adapted for inhaled administration, the pharmaceutical composition may be a dry powder composition or an aerosol formulation, comprising a solution or fine suspension of the active substance in a pharmaceutically acceptable aqueous or non-aqueous solvent. Dry powder compositions can comprise a powder base such as lactose, glucose, trehalose, mannitol or starch, the compounds of formula (I) or a pharmaceutically acceptable salt thereof (preferably in particle-size-reduced form, e.g. in micronised form), and optionally a performance modifier such as L-leucine or another amino acid and/or metal salt of stearic acid such as magnesium or calcium stearate. Preferably, the dry powder inhalable composition comprises a dry powder blend of lactose e.g. lactose monohydrate and the compound of formula (I) or a salt thereof.

In one embodiment, a dry powder composition suitable for inhaled administration may be incorporated into a plurality of sealed dose containers provided on medicament pack(s) mounted inside a suitable inhalation device. The containers may be rupturable, peelable or otherwise openable one-at-a-time and the doses of the dry powder composition administered by inhalation on a mouthpiece of the inhalation device, as known in the art. The medicament pack may take a number of different forms, for instance a disk-shape or an elongate strip. Representative inhalation devices are the DISKHALER™ inhaler device, the DISKUS™ inhalation device, and the ELLIPTA™ inhalation device, marketed by GlaxoSmithKline. The DISKUS™ inhalation device is, for example, described in GB 2242134A, and the ELLIPTA™ inhalation device is, for example, described in WO 2003/061743 A1, WO 2007/012871 A1 and/or WO 2007/068896 A1.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical compositions adapted for topical administration may be formulated as ointments, creams, suspensions, emulsions, lotions, powders, solutions, pastes, gels, foams, sprays, aerosols or oils. Such pharmaceutical compositions may include conventional additives which include, but are not limited to, preservatives, solvents to assist drug penetration, co-solvents, emollients, propellants, viscosity modifying agents (gelling agents), surfactants and carriers. In one embodiment there is provided a pharmaceutical composition adapted for topical administration which comprises between 0.01-10%, or between 0.01-1% of a compound of formula (I)-(XVI), or a pharmaceutically acceptable salt thereof, by weight of the composition.

For treatments of the eye or other external tissues, for example mouth and skin, the compositions are preferably applied as a topical ointment, cream, gel, spray or foam. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administrations to the eye include eye drops wherein the active ingredient is dissolved or suspended in a suitable carrier, especially an aqueous solvent.

A therapeutically effective amount of a compound of formula (I) or a pharmaceutically acceptable salt thereof, will depend upon a number of factors including, for example, the age and weight of the subject, the precise condition requiring treatment and its severity, the nature of the formulation, and the route of administration, and will ultimately be at the discretion of the attendant physician or veterinarian. In the pharmaceutical composition, each dosage unit for oral administration preferably contains from 0.01 to 1000 mg, more preferably 0.5 to 100 mg, of a compound of formula (I) or a pharmaceutically acceptable salt thereof, calculated as the free base.

The compounds of formula (I) and pharmaceutically acceptable salts thereof may be employed alone or in combination with other therapeutic agents. Combination therapies according to the present invention thus comprise the administration of at least one compound of formula (I) or a pharmaceutically acceptable salt thereof, and the use of at least one other therapeutically active agent. A compound of formula (I) or pharmaceutically acceptable salt thereof, and the other therapeutically active agent(s) may be administered together in a single pharmaceutical composition or separately and, when administered separately this may occur simultaneously or sequentially in any order.

In a further aspect, there is provided a combination product comprising a compound of formula (I) or a pharmaceutically acceptable salt thereof, together with one or more other therapeutically active agents, and optionally one or more pharmaceutically acceptable excipients.

It will be clear to a person skilled in the art that, where appropriate, the other therapeutic ingredient(s) may be used in the form of salts, for example as alkali metal or amine salts or as acid addition salts, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable excipient.

### General Synthetic Routes

The compounds of formula (I) and salts thereof may be prepared by the methodology described hereinafter, constituting further aspects of this invention:

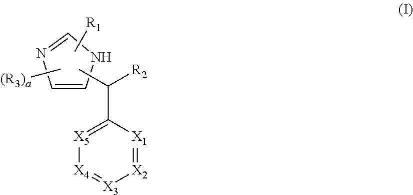

wherein R₁, R₂, R₃, X₁, X₂, X₃, X₄, X₅, and a are as hereinbefore defined.

Accordingly, there is provided a process for the preparation of a compound of formula (IIa):

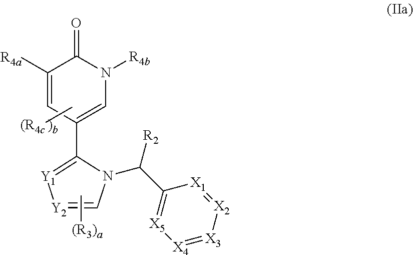

which process comprises the cross-coupling of a compound of formula (III):

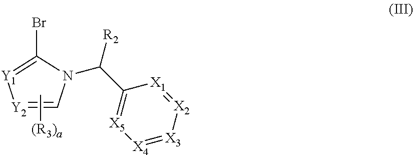

wherein R₂, R₃, X₁, X₂, X₃, X₄, X₅, and a are as defined hereinbefore for a compound of formula (I). Y₁ and Y₂ represent CH or N provided that when Y₁ is CH, Y₂ is N and vica versa. For example, a compound of formula (III) could be dissolved in a suitable solvent mixture, such as 1,4-dioxane/water, then treated with a suitable coupling partner of formula (IV) in the presence of a palladium catalyst with heating at a suitable temperature for an appropriate time to give, after purification, compounds of the formula (IIa). The coupling partners are of general formula (IV) wherein R₄ₐ, R_{4b}, R_{4c} and b are as defined for a compound of formula (I).

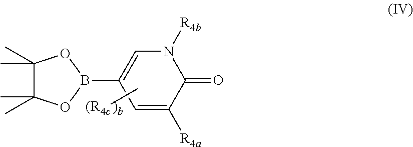

There is further provided a process for the preparation of a compound of formula (V):

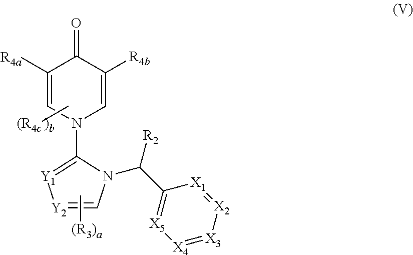

which process comprises the cross-coupling of a compound of formula (III) above. A compound of formula (III) could, for example, be dissolved in a suitable solvent, such as dimethyl sulfoxide, and then treated with a suitable coupling partner of formula (VI) in the presence of a copper catalyst with heating at a suitable temperature for an appropriate time to give, after purification, a compound of the formula (V).

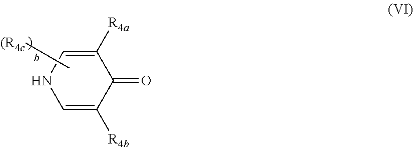

There is provided a process for the preparation of a compound of formula (III), which process comprises the bromination of a compound of formula (VII):

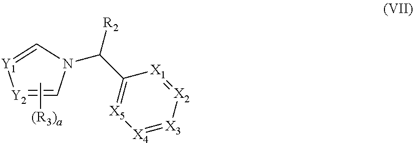

wherein R₂, R₃, X₁, X₂, X₃, X₄, X₅, and a are as defined hereinbefore for a compound of formula (I). Y₁ and Y₂ represent CH or N provided that when Y₁ is CH, Y₂ is N and vica versa. For example, a compound of formula (VII) could be dissolved in a suitable solvent, such as THF, then treated with a suitable base, for example TMPMgCl.LiCl, followed by a brominating agent, such as CBr₄. The mixture would then be stirred at a suitable temperature for an appropriate time to give, after purification, compounds of the formula (III).

Alternatively, there is provided a process for the preparation of a compound of formula (IIa)

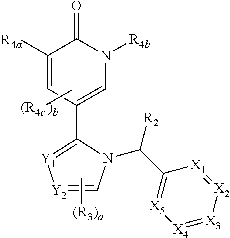

which process comprises the alkylation of a compound of formula (VIII):

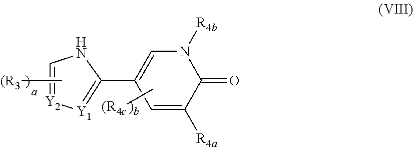

Wherein R₃, R₄ and a are as defined hereinbefore for a compound of formula (I). Y₁ and Y₂ represent CH or N provided that when Y₁ is CH, Y₂ is N and vica versa. For example, a compound of formula (VIII) could be dissolved in a suitable solvent, such as DMF, then treated with a suitable base, such as sodium hydride, in the presence of an benzyl halide or heteroarylmethyl halide and heated at a suitable temperature for an appropriate time to give, after purification, compounds of the formula (II) wherein R₂, R₃, R₄ₐ, R_{4b}, R_{4c}, X₁, X₂, X₃, X₄, X₅, a and b are as defined hereinbefore for a compound of formula (I).

There is further provided a process for the preparation of a compound of formula (IIb):

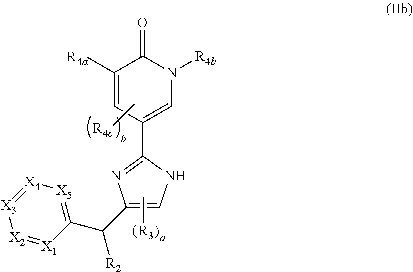

which process comprises the alkylation of a compound of formula (VIII):

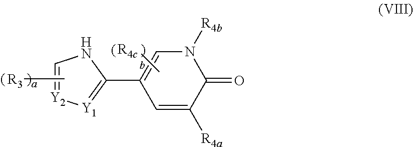

wherein R₃, R₄ₐ, R_{4b}, R_{4c}, b and a are as defined hereinbefore for a compound of formula (I), and Y₁ is N and Y₂ is CH. For example, a compound of formula (VIII) is dissolved in a suitable solvent, such as dimethylsulfoxide, then treated with suitable reagents, such as Ir(ppy)₂(dtbbpy)PF₆, tosic acid and methyl thioglycolate in the presence of an alcohol and irradiated with blue light at a suitable temperature for an appropriate time to give, after purification, compounds of formula (IIb) wherein R₂, R₃, R₄ₐ, R_{4b}, R_{4c}, b and a are as defined hereinbefore for a compound of formula (I).

There is further provided a process for the preparation of a compound of formula (IIc):

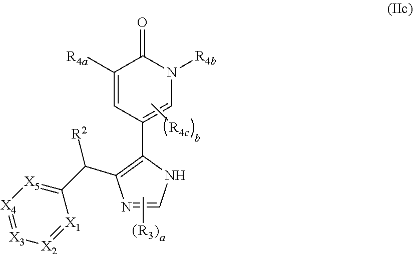

which process comprises the alkylation of a compound of formula (VIII):

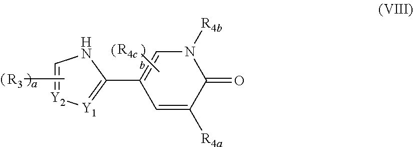

wherein R₃, R₄ₐ, R_{4b}, R_{4c}, b and a are as defined hereinbefore for a compound of formula (I), and Y₁ is CH and Y₂ is N. For example, a compound of formula (VIII) is dissolved in a suitable solvent, such as dimethylsulfoxide, then treated with suitable reagents, such as Ir(ppy)₂(dtbbpy)PF₆, tosic acid and methyl thioglycolate in the presence of an alcohol and irradiated with blue light at a suitable temperature for an appropriate time to give, after purification, compounds of the formula (IIc) wherein R₂, R₃, R₄ₐ, R_{4b}, R_{4c}, b and a are as defined hereinbefore for a compound of formula (I).

There is further provided a process for the preparation of a compound of formula (IId):

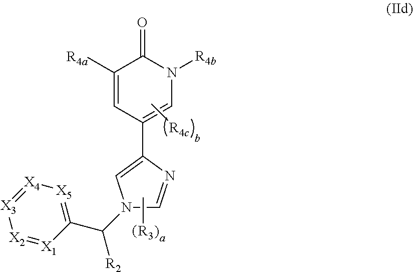

which process comprises the alkylation of a compound of formula (VIII):

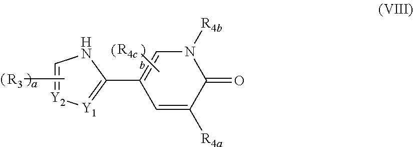

wherein R₃, R₄ₐ, R_{4b}, R_{4c}, b and a are as defined hereinbefore for a compound of formula (I), and Y₁ is CH and Y₂ is N. For example, a compound of formula (VIII) is dissolved in a suitable solvent, such as N,N-dimethylformamide, then treated with a suitable base in the presence of an alkyl halide and heated at a suitable temperature for an appropriate time to give, after purification, compounds of the formula (IId) wherein R₂, R₃, R₄ₐ, R_{4b}, R_{4c}, b and a are as defined hereinbefore for a compound of formula (I).

There is provided a process for the preparation of a compound of formula (VIII), which process comprises cross-coupling of a compound of formula (III):

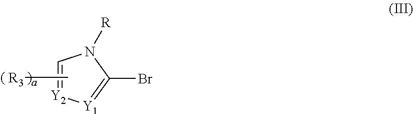

wherein R₃ and a are defined hereinbefore for a compound of formula (I) and R is optionally a proton or suitable protecting group (for example [2-(trimethylsilyl)ethoxy]methyl acetal). Y₁ and Y₂ represent CH or N provided that when Y₁ is CH, Y₂ is N and vica versa. For example, a compound of formula (III) could be dissolved in a suitable solvent mixture, such as 1,4-dioxane/water, then treated with a suitable coupling partner of formula (IV) in the presence of a palladium catalyst with heating at a suitable temperature for an appropriate time to give, after purification, compounds of the formula (VIII) post suitable deprotection (for example using TBAF) as appropriate. The coupling partners mentioned above are of general formula (IV) wherein R₄ is defined for a compound of formula (I).

There is provided a process for the preparation of a compound of formula (I), which process comprises functionalisation of a compound of the formula (I) where R₃ is a suitable functional group (for example an ester). Such compounds may be functionalised, for example by hydrolysis and, where appropriate, further coupling with, for example a suitable amine in the presence of a suitable coupling agent, such as HATU, to give compounds of formula (I) wherein R₂, R₃ and R₄ are as defined hereinbefore for a compound of formula (I).

Compounds of formula III, IV, VI and VII are commercially available from, for example, Sigma Aldrich.

### EXPERIMENTAL

### Abbreviations

CV Column volumes
DCM Dichloromethane
DIPEA N,N-diisopropylethylamine
DMF N,N-dimethylformamide
DMSO Dimethylsulfoxide
g Grammes
h Hour(s)
EtOAc Ethyl acetate
HATU N,N,N′,N′-Tetramethyl-O-(7-azabenzotriazol-1-yl)uronium hexafluorophosphate
HPLC High-performance liquid chromatography
L Litre
LCMS Liquid chromatography-mass spectrometry
MDAP Mass-Directed Auto-Preparative HPLC
MeOH Methanol
min Minutes
mg Milligrammes
MHz Megahertz
mL Millilitre
mM Millimolar
nm Nanometre
ppm Parts per million
THF Tetrahydrofuran
TMPMgCl.LiCl 2,2,6,6-Tetramethylpiperidinylmagnesium chloride lithium chloride complex
t_{RET} Retention time
μm Micrometre

### Experimental Details

### LCMS

### System A:

The UPLC analysis was conducted on an Acquity UPLC CSH C18 column (50 mm×2.1 mm i.d. 1.7 μm packing diameter) at 40° C.
The solvents employed were:
A=0.1% v/v solution of formic acid in water.
B=0.1% v/v solution of formic acid in MeCN.
The gradient employed was:

| Time (min) | Flow (mL/min) | % A | % B |
|---|---|---|---|
| 0 | 1 | 97 | 3 |
| 1.5 | 1 | 5 | 95 |
| 1.9 | 1 | 5 | 95 |
| 2.0 | 1 | 97 | 3 |

The UV detection was a summed signal from wavelength of 210 nm to 350 nm.
Injection volume: 0.5 μL

### MS Conditions

### MS: Waters ZQ

Ionisation mode: Alternate-scan Positive and Negative Electrospray

### Scan Range: 100 to 1000 AMU

### Scan Time: 0.27 s

Inter scan Delay: 0.10 s

### System B:

The UPLC analysis was conducted on an Acquity UPLC CSH C18 column (50 mm×2.1 mm i.d. 1.7 μm packing diameter) at 40° C.
The solvents employed were:
A=10 mM ammonium bicarbonate in water adjusted to pH 10 with ammonia solution.

### B=MeCN.

The gradient employed was:

| Time (min) | Flow (mL/min) | % A | % B |
|---|---|---|---|
| 0 | 1 | 97 | 3 |
| 0.05 | 1 | 97 | 3 |
| 1.50 | 1 | 5 | 95 |
| 1.90 | 1 | 5 | 95 |
| 2.00 | 1 | 97 | 3 |

The UV detection was a summed signal from wavelength of 210 nm to 350 nm.
Injection volume: 0.3 μL

### MS Conditions

### MS: Waters ZQ

Ionisation mode: Alternate-scan Positive and Negative Electrospray

### Scan Range: 100 to 1000 AMU

### Scan Time: 0.27 s

Inter scan Delay: 0.10 s

### System C:

The UPLC analysis was conducted on an Acquity UPLC CSH C18 column (50 mm×2.1 mm i.d. 1.7 μm packing diameter) at 40° C.
The solvents employed were:
A=0.1% v/v trifluoroacetic acid in water.
B=0.1% v/v trifluoroacetic acid in MeCN.
The gradient employed was:

| Time (min) | Flow (mL/min) | % A | % B |
|---|---|---|---|
| 0 | 1 | 95 | 5 |
| 1.50 | 1 | 5 | 95 |
| 1.90 | 1 | 5 | 95 |
| 2.00 | 1 | 95 | 5 |

The UV detection was a summed signal from wavelength of 210 nm to 350 nm.
Injection volume: 0.5 μL

### MS Conditions

### MS: Waters ZQ

Ionisation mode: Positive Electrospray

### Scan Range: 100 to 1000 AMU

### Scan Time: 0.27 s

Inter scan Delay: 0.05 s

### System D:

The UPLC analysis was conducted on an Xbridge C18 column (50 mm×4.6 mm i.d. 2.5 μm packing diameter) at 35° C.
The solvents employed were:
A=5 mM Ammonium Bicarbonate in water (pH 10).

### B=Acetonitrile

The gradient employed was:

| Time (min) | Flow (mL/min) | % A | % B |
|---|---|---|---|
| 0 | 1.3 | 95 | 5 |
| 0.5 | 1.3 | 95 | 5 |
| 1.0 | 1.3 | 85 | 15 |
| 3.3 | 1.3 | 2 | 98 |
| 5.2 | 1.3 | 2 | 98 |
| 5.5 | 1.3 | 95 | 5 |
| 6.0 | 1.3 | 95 | 5 |

The UV detection was a summed signal from wavelength of 200 nm to 400 nm.
Injection volume: 3.0 μL

### MS Conditions

MS: Waters Quattro micro
Ionisation mode: Alternate-scan Positive and Negative Electrospray

### Scan Range: 100 to 1000 AMU

### Scan Time: 0.50 s

Inter scan Delay: 0.10 s

### System E:

The UPLC analysis was conducted on an Acquity BEH C18 column (100 mm×2.1 mm i.d. 1.7 μm packing diameter) at 35° C.
The solvents employed were:
A=0.05% v/v solution of formic acid in acetonitrile
B=0.05% v/v solution of formic acid in water
The gradient employed was:

| Time (min) | Flow (mL/min) | % A | % B |
|---|---|---|---|
| 0 | 0.45 | 3 | 97 |
| 0.4 | 0.45 | 3 | 97 |
| 7.5 | 0.45 | 98 | 2 |
| 9.5 | 0.45 | 98 | 2 |
| 9.6 | 0.45 | 3 | 97 |
| 10.0 | 0.45 | 3 | 97 |

The UV detection was a summed signal from wavelength of 200 nm to 400 nm.
Injection volume: 0.5 μL

### MS Conditions

### MS: Waters XEVO-TQS

Ionisation mode: Alternate-scan Positive and Negative Electrospray

### Scan Range: 100 to 1000 AMU

### Scan Time: 0.50 s

Inter scan Delay: Automatic

### Mass Directed Autopreparative HPLC (MDAP)

Mass directed autopreparative HPLC was undertaken under the conditions given below. The UV detection was an averaged signal from wavelength of 210 nm to 350 nm and mass spectra were recorded on a mass spectrometer using alternate-scan positive and negative mode electrospray ionization.

### Method A

Method A was conducted on an Xselect CSH C18 column (typically 150 mm×30 mm i.d. 5 μm packing diameter) at ambient temperature. The solvents employed were:
A=0.1% v/v solution of formic acid in water.
B=0.1% v/v solution of formic acid in acetonitrile.

### Method B

Method B was conducted on an Xselect CSH C18 column (typically 150 mm×30 mm i.d. 5 μm packing diameter) at ambient temperature. The solvents employed were:
A=10 mM Ammonium bicarbonate in water adjusted to pH 10 with Ammonia

### B=Acetonitrile.

### Method C

Method C was conducted on an Xselect CSH column (typically 150 mm×30 mm i.d. 5 μm packing diameter) at ambient temperature. The solvents employed were:
A=0.1% v/v solution of TFA in water
B=0.1% v/v solution of TFA in acetonitrile.

### Intermediate Preparation

Unless otherwise stated, starting materials for the preparation of Intermediates and Examples are commercially available from, for example, Milestone PharmaTech and Sigma Aldrich.

### Intermediate 1: 3-((2-bromo-1H-imidazol-1-yl)methyl)pyridine

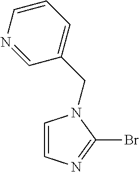 [SMILES: Brc1nccn1Cc1cccnc1]

To 2-bromo-1H-imidazole (0.073 g, 0.5 mmol), 3-(bromomethyl)pyridine hydrobromide (0.139 g, 0.550 mmol) and potassium carbonate (0.223 g, 1.614 mmol) was added acetone (15 mL). This mixture was stirred for 18 h at RT. The solvent was then removed in vacuo yielding a brown residue. This residue was then partitioned between ethyl acetate (35 mL) and water (35 mL) and the phases separated. The aqueous phase was then extracted twice with ethyl acetate (2×35 mL), the organics combined and then dried using a hydrophobic frit. The solvent was then removed in vacuo yielding a yellow oil as the title compound (107 mg, 0.449 mmol, 90%). LCMS (System B): t_{RET}=0.56 min; MH⁺ 238, 240.

### Intermediate 2: 4-((2-bromo-1H-imidazol-1-yl)methyl)pyridine

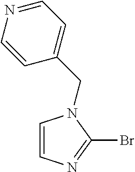 [SMILES: Brc1nccn1Cc1ccncc1]

To 2-bromo-1H-imidazole (73.5 mg, 0.5 mmol), 4-(bromomethyl)pyridine hydrobromide (139 mg, 0.550 mmol) and potassium carbonate (207 mg, 1.500 mmol) was added acetone (15 mL). This mixture was stirred for 18 h at RT. The solvent was then removed in vacuo yielding a brown residue. This residue was then partitioned between ethyl acetate (35 mL) and water (35 mL) and the phases separated. The aqueous phase was then extracted twice with ethyl acetate (2×35 mL), the organics combined and then dried using a hydrophobic frit. The solvent was then removed in vacuo yielding a dark brown oil determined to be the title compound (108 mg, 0.454 mmol, 91%). LCMS (System B): t_{RET}=0.57 min; MH⁺ 238, 240.

### Intermediate 3: 2-((2-bromo-1H-imidazol-1-yl)methyl)pyridine

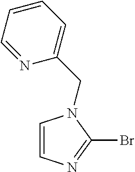 [SMILES: Brc1nccn1Cc1ccccn1]

2-Bromo-1H-imidazole (100 mg, 0.680 mmol) was dissolved in DMF (2 mL) in a microwave vial in an ice bath and purged with nitrogen for 10 min. With the solution at 0° C., sodium hydride (60% dispersion in mineral oil) (62.6 mg, 1.565 mmol) was added and the solution was stirred for 10 min under nitrogen. A solution of 2-(bromomethyl)pyridine hydrobromide (207 mg, 0.816 mmol) in DMF (2 mL) was added dropwise to the reaction mixture. Once all reagents were added, the reaction mixture was allowed to warm to RT and stirred for 2 days under an atmosphere of nitrogen. The solvent was removed under reduced pressure. The crude product was dissolved in ethyl acetate (20 mL) and washed with brine (20 mL). The solvent was again removed from the organic layer under reduced pressure. The samples were dissolved in 1:1 MeOH:DMSO (3 mL) and purified by MDAP (Method B). The solvent was evaporated in vacuo to give the title compound. LCMS (System B): t_{RET}=0.63 min; MH⁺ 238, 240.

### Intermediate 4: 5-(1H-imidazol-2-yl)-1,3-dimethylpyridin-2(1H)-one

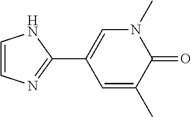

To a degassed solution of 2-bromo-1H-imidazole (21.0 g, 143 mmol), 1,3-dimethyl-5-(4,4,5,5-tetramethyl-1,3,2-dioxaborolan-2-yl)pyridin-2(1H)-one (commercially available from, for example, Milestone PharmaTech, 38.0 g, 152 mmol) and potassium carbonate (57.4 g, 415 mmol) in 1,4-dioxane (200 mL) and water (60 mL) stirred under nitrogen at RT was added solid Tetrakis(triphenylphosphine)palladium(0) (8.00 g, 6.92 mmol) in one charge. The reaction mixture was stirred at 100° C. for 16 h. The reaction mixture was filtered through a Celite® pad and the filterate was separated. The aqueous layer was re-extracted with 10% MeOH in DCM (2×100 mL). The combined organic layers were washed with brine solution (100 mL), dried over sodium sulphate, filtered and evaporated in vacuo to give the crude product as a brown gum. The crude product was triturated with 10% DCM in diethyl ether (2×50 mL). The resultant solid was filtered and dried under reduced pressure to afford crude compound as cream solid. This compound was triturated with diethylether and filtered through a Celite® pad and dried under reduced pressure to afford the title compound (23.0 g, 121.7 mmol, 85%) as cream colored solid. LCMS (System D): t_{RET}=2.14 min; MH⁺ 190.

### Intermediate 5: methyl 1-((2-(trimethylsilyl)ethoxy)methyl)-1H-imidazole-4-carboxylate

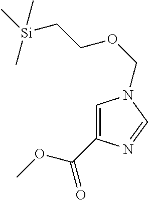 [SMILES: COC(=O)c1cn(COCC[Si](C)(C)C)cn1]

Methyl 1H-imidazole-4-carboxylate (2 g, 15.86 mmol) and potassium carbonate (4.38 g, 31.7 mmol) were added to a round bottomed flask containing a stirrer bar and placed under an atmosphere of nitrogen by evacuation-refill. Acetone (20 mL) was added, evacuation-refill of the vessel repeated, and the mixture stirred prior to addition of (2-(chloromethoxy)ethyl)trimethylsilane (3.37 mL, 19.03 mmol). The reaction vessel was placed under an atmosphere of nitrogen and left stirring overnight at RT. A further 0.33 equivalents of (2-(chloromethoxy)ethyl)trimethylsilane (0.926 mL, 5.23 mmol) were added and the reaction left to continue for a further 4 h. The reaction mixture was quenched with addition of 40 mL water and extracted with EtOAc (40 mL), with the addition of 10 mL brine to prevent formation of a triphasic solution. The aqueous layer was extracted with a further 3×40 mL EtOAc. The organic layers were combined, passed through a hydrophobic frit, and the solvent removed under reduced pressure. The sample was dissolved in DCM and purified by flash chromatography using a silica 120 g cartridge, using a solvent system of 10-75% ethyl acetate-cyclohexane over 25 CV. The appropriate fractions were combined and evaporated in vacuo to give:

methyl 1-((2-(trimethylsilyl)ethoxy)methyl)-1H-imidazole-5-carboxylate (1.45 g). LCMS (System B): t_{RET}=1.10 min; MH⁺ 257.

methyl 1-((2-(trimethylsilyl)ethoxy)methyl)-1H-imidazole-4-carboxylate (the title compound) (1.34 g). LCMS (System B): t_{RET}=1.02 min; MH⁺ 257.

### Intermediate 6: methyl 2-bromo-1-((2-(trimethylsilyl)ethoxy)methyl)-1H-imidazole-4-carboxylate

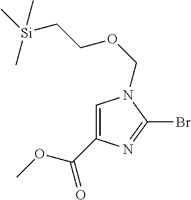 [SMILES: COC(=O)c1cn(COCC[Si](C)(C)C)c(Br)n1]

Methyl 1-((2-(trimethylsilyl)ethoxy)methyl)-1H-imidazole-4-carboxylate (for an example preparation, see Intermediate 5, 297 mg, 1.158 mmol) was added to a round bottomed flask containing trifluorotoluene (6 mL). Once dissolved, azobisisobutyronitrile (9.51 mg, 0.058 mmol) and N-bromosuccinimide (227 mg, 1.274 mmol) were added, and the flask placed under an atmosphere of nitrogen. The reaction mixture was stirred at 65° C. overnight. The reaction mixture was quenched with saturated sodium hydrogencarbonate solution (20 mL) and extracted with EtOAc (2×20 mL). The organic layers were combined and the solvent removed under reduced pressure. The sample was loaded in DCM and purified by column chromatography using a silica cartridge (80 g) with an ethyl acetate-cyclohexane solvent system [10-20%, 1CV; 20%, 7CV; 20-100%, 3CV; 100%, 3CV]. The appropriate fractions were combined and the solvent removed in vacuo to afford the title compound as a white solid (206 mg, 0.61 mmol, 53%). LCMS (System B): t_{RET}=1.16 min; MH⁺ 335.

### Intermediate 7: methyl 2-(1,5-dimethyl-6-oxo-1,6-dihydropyridin-3-yl)-1-((2-(trimethylsilyl)ethoxy)methyl)-1H-imidazole-4-carboxylate

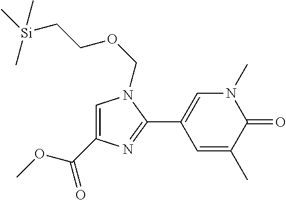 [SMILES: COC(=O)c1cn(COCC[Si](C)(C)C)c(-c2cc(C)c(=O)n(C)c2)n1]

1,3-Dimethyl-5-(4,4,5,5-tetramethyl-1,3,2-dioxaborolan-2-yl)pyridin-2(1H)-one (1.739 g, 6.98 mmol), methyl 2-bromo-1-((2-(trimethylsilyl)ethoxy)methyl)-1H-imidazole-4-carboxylate (for an example preparation, see Intermediate 6, 1.56 g, 4.65 mmol), and potassium carbonate (1.929 g, 13.96 mmol) were added to a 5 mL microwave vial containing a stirrer bar. 1,4-Dioxane (15 mL) and methanol (5 mL) were added to the vial, which was purged with nitrogen for 5 mins prior to the addition of tetrakis(triphenylphosphine)palladium(0) (0.161 g, 0.140 mmol). After a further 5 min purge with nitrogen, the vial was capped and heated in the microwave at 100° C. for 1 h. A further 0.5 equivalents of 1,3-dimethyl-5-(4,4,5,5-tetramethyl-1,3,2-dioxaborolan-2-yl)pyridin-2(1H)-one (commercially available from, for example, Milestone PharmaTech, 0.580 g, 2.326 mmol) and 1 mol % of tetrakis(triphenylphosphine)palladium(0) (0.054 g, 0.047 mmol) were added to the microwave vial, which was purged with nitrogen for a further 10 min and returned to the microwave for another 1 h of heating at 100° C. The solvent from the reaction mixture was removed by evaporation under reduced pressure. The residue was redissolved in ethyl acetate and filtered through Celite®. The solvent was removed under reduced pressure. The sample was loaded in DCM and purified by column chromatography using a silica cartridge (120 g) with an ethyl acetate-cyclohexane solvent system [25-75%, 15CV; 75%, 10CV]. The appropriate fractions were combined and the solvent removed in vacuo to give the crude product. The crude product was redissolved in ethyl acetate (30 mL) and washed with 8 portions of water/brine (30 mL/10 mL) until all traces of impurity had been removed from the organic layer. The organic layer was passed through a hydrophobic frit and the solvent removed under reduced pressure to yield the title compound as a beige solid, (1.76 g). LCMS (System B): t_{RET}=1.06 min; MH⁺ 378.

### Intermediate 8: methyl 2-(1,5-dimethyl-6-oxo-1,6-dihydropyridin-3-yl)-1H-imidazole-4-carboxylate

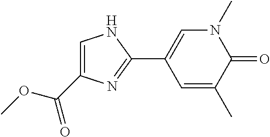 [SMILES: COC(=O)c1c[nH]c(-c2cc(C)c(=O)n(C)c2)n1]

Methyl 2-(1,5-dimethyl-6-oxo-1,6-dihydropyridin-3-yl)-1-((2-(trimethylsilyl)ethoxy)methyl)-1H-imidazole-4-carboxylate (for an example preparation, see Intermediate 7, 1.76 g, 4.66 mmol) was added to a round bottomed flask containing a stirrer bar and dissolved in anhydrous methanol (20 mL). The flask was purged with nitrogen by evacuation-refill, and trimethylsilylchloride (11.92 mL, 93 mmol) added to the reaction mixture. The reaction mixture was stirred at 40° C. for 18 h under an atmosphere of nitrogen. The solvent was removed under reduced pressure, and the crude product twice redissolved in methanol (30 mL) and the solvent removed in vacuo. The crude product was loaded in methanol and purified by SPE using a 20 g sulphonic acid (SCX) catridge, with sequential solvent elution of methanol followed by 2M ammonia in methanol. The appropriate fractions were combined and the solvent removed in vacuo to give the title compound as a white solid, (773 mg, 3.13 mmol, 67%). LCMS (System B): t_{RET}=0.56 min; MH⁺ 248.

### Intermediate 9: 4-chloro-1-((2-(trimethylsilyl)ethoxy)methyl)-1H-imidazole

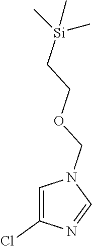

4-Chloro-1H-imidazole (2 g, 19.51 mmol) and potassium carbonate (5.39 g, 39.0 mmol) were added to a round bottomed flask containing a stirrer bar, followed by addition of DMF (25 mL). The reaction mixture was placed under an atmosphere of nitrogen by evacuation-refill, and a solution of (2-(chloromethoxy)ethyl)trimethylsilane (6.91 mL, 39.0 mmol) in DMF (25 mL) added dropwise, before stirring at RT for 3.5 h under an atmosphere of nitrogen. The reaction mixture was quenched with 20 mL water, and the solvents removed under reduced pressure. The residue was dissolved in 50 mL EtOAc, and washed with 30 mL water, then 30 mL brine. The organic layer was passed through a hydrophobic frit and the solvent removed under reduced pressure. The sample was loaded in a minimum of DCM and purified by gradient elution column chromatography using a 120 g silica (Si) cartridge eluting with an ethyl acetate-cyclohexane solvent system [0%, 1CV; 0-30%, 15CV]. The appropriate fractions were combined and the solvent removed in vacuo to give the title compound as a pale yellow oil (2.37 g, 10.2 mmol, 52%). LCMS (System B): t_{RET}=1.15 min; MH⁺ 233, 235.

### Intermediate 10: 2-bromo-4-chloro-1-((2-(trimethylsilyl)ethoxy)methyl)-1H-imidazole

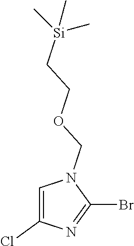

A solution of 4-chloro-1-((2-(trimethylsilyl)ethoxy)methyl)-1H-imidazole (for an example preparation, see Intermediate 9, 2 g, 8.59 mmol) in THF (22 mL) was placed under an atmosphere of nitrogen and cooled to 0° C. A solution of TMPMgCl.LiCl (1M in THF/toluene, 12.89 mL, 12.89 mmol) was added dropwise and the reaction mixture stirred for 1 h at 0° C. under nitrogen. A solution of CBr₄ (5.70 g, 17.18 mmol) in THF (20 mL) was added dropwise over 5 min, and the reaction mixture was allowed to warm to RT. Stirring was continued for a further 3 h under nitrogen. The reaction was quenched by the addition of saturated sodium hydrogen carbonate solution (5 mL) and extracted with DCM (3×5 mL). The combined organic layers were passed through a hydrophobic frit and the solvent removed in vacuo. The crude sample was dissolved in DCM (10 mL) and purified by flash column chromatography using an elution gradient of 0-30% EtOAc in cyclohexane over 30 CV (120 g silica cartridge). The appropriate fractions were combined and the solvent removed in vacuo to afford the title compound as a light brown oil (1.86 g, 5.96 mmol, 6669% yield). LCMS (System A): t_{RET}=1.30 min; MH⁺ 311, 313, 315.

### Intermediate 11: 5-(4-chloro-1-((2-(trimethylsilyl)ethoxy)methyl)-1H-imidazol-2-yl)-1,3-dimethylpyridin-2(1H)-one

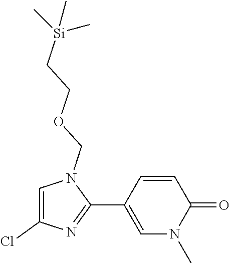 [SMILES: Cn1cc(-c2nc(Cl)cn2COCC[Si](C)(C)C)ccc1=O]

Potassium carbonate (1 g, 7.24 mmol), 1,3-dimethyl-5-(4,4,5,5-tetramethyl-1,3,2-dioxaborolan-2-yl)pyridin-2(1H)-one (0.863 g, 3.47 mmol) and 2-bromo-4-chloro-1-((2-(trimethylsilyl)ethoxy)methyl)-1H-imidazole (for an example preparation, see Intermediate 10, 0.9 g, 2.89 mmol), 1,4-dioxane (10 mL) and water (2.5 mL) were each added to 2×20 mL microwave vials and the reaction mixtures purged with nitrogen for 5 min. Tetrakis(triphenylphosphine)palladium(0) (0.100 g, 0.087 mmol) was added to each, the vials sealed, and purged with nitrogen for a further 5 min. The microwave vials were each heated to 110° C. in a Biotage I60 microwave reactor for 1 h. The two vials were combined and the solvent was removed in vacuo. The crude residue was redissolved in EtOAc (20 mL) and filtered through Celite®, washing with further EtOAc (3×20 mL). The solvent was removed in vacuo once more. The crude sample was dissolved in DCM (10 mL) and purified by flash column chromatography using an elution gradient of 0-100% EtOAc in cyclohexane over 30 CV (120 g silica cartridge). The appropriate fractions were combined and the solvent removed in vacuo to afford the title compound in two batches, both as light yellow solids (921 mg, 2.60 mmol, 45%; 538 mg, 1.44 mmol, 25%). LCMS (System A): t_{RET}=1.16 min; MH⁺ 354, 356.

### Intermediate 12: 5-(4-chloro-1H-imidazol-2-yl)-1,3-dimethylpyridin-2(1H)-one

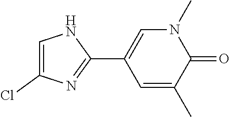

5-(4-chloro-1-((2-(trimethylsilyl)ethoxy)methyl)-1H-imidazol-2-yl)-1,3-dimethylpyridin-2(1H)-one (for an example preparation, see Intermediate 11, 538 mg, 1.52 mmol) was added to a round bottomed flask containing a stirrer bar and dissolved in anhydrous methanol (6 mL). The reaction mixture was purged with nitrogen by evacuation-refill, and trimethylsilylchloride (3.8 mL, 29.7 mmol) was added. The reaction mixture was stirred at 40° C. overnight. Further trimethylsilylchloride (3.8 mL, 29.7 mmol) was added to the reaction mixture and the reaction was left to stir at 40° C. for a further day. The solvent was removed from the reaction mixture under reduced pressure. The crude product was loaded in methanol and purified by SPE using a 2 g sulphonic acid (SCX) catridge, with sequential solvent elution of methanol followed by 2M ammonia in methanol. The appropriate fractions were combined and the solvent removed in vacuo to give the title compound (324 mg, 1.45 mmol, 95%). LCMS (System A): t_{RET}=0.57 min; MH⁺ 224, 226.

### Intermediate 13: lithium 1-benzyl-2-(1,5-dimethyl-6-oxo-1,6-dihydropyridin-3-yl)-1H-imidazole-4-carboxylate

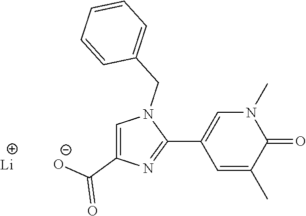 [SMILES: [Li+] ; Cc1cc(-c2nc(C(=O)[O-])cn2Cc2ccccc2)cn(C)c1=O]

Methyl 1-benzyl-2-(1,5-dimethyl-6-oxo-1,6-dihydropyridin-3-yl)-1H-imidazole-4-carboxylate (for an example preparation, see Example 10, 450 mg, 1.334 mmol) was dissolved in methanol (10 mL) prior to addition of lithium hydroxide (55 mg, 2.297 mmol). The reaction mixture was stirred at RT overnight under an atmosphere of nitrogen. The reaction temperature was increased to 50° C., and the reaction mixture stirred for a further 8 h. The reaction temperature was increased to 60° C., and the reaction mixture stirred overnight. The solvent was removed under reduced pressure to afford the title compound as an off-white solid (427 mg, 1.30 mmol, 97%). LCMS (System B): t_{RET}=0.51 min; MH±(free acid) 324.

### Intermediate 14: 1-benzyl-2-bromo-4-nitro-1H-imidazole

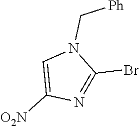 [SMILES: O=[N+]([O-])c1cn(Cc2ccccc2)c(Br)n1]

To a stirred solution of 2-bromo-5-nitro-1H-imidazole (1380 mg, 7.19 mmol) in DMF (10 mL) at 0° C. was added sodium hydride (60% dispersion in mineral oil) (431 mg, 10.78 mmol). The reaction was stirred for 30 min, then benzyl bromide (1.283 mL, 10.78 mmol) was added. The reaction was allowed to warm to RT and stirred for a further 16 h. The reaction was quenched with MeOH (5 mL) and the solvent removed in vacuo. The residue was suspended in EtOAc (20 mL) and filtered through Celite®, washing with EtOAc (3×20 mL), and the solvent was removed in vacuo. Purification by MDAP (Method B) afforded the title compound as a light yellow solid (1237 mg, 4.39 mmol, 61%). LCMS (System B): t_{RET}=1.00 min; MH⁺ 282, 284.

### Intermediate 15: 1-((2-(trimethylsilyl)ethoxy)methyl)-1H-imidazole-4-carbonitrile

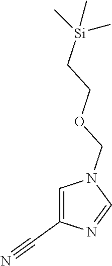

1H-imidazole-4-carbonitrile (1 g, 10.74 mmol) and potassium carbonate (2.97 g, 21.49 mmol) were added to a round bottomed flask and placed under an atmosphere of nitrogen by evacuation-refill. Acetone (10 mL) was added, evacuation-refill of the vessel repeated, and the mixture stirred prior to addition of (2-(chloromethoxy)ethyl)trimethylsilane (2.091 mL, 11.82 mmol). The reaction vessel was placed under an atmosphere of nitrogen and stirred at RT for 48 h. The solvent was removed under reduced pressure, and the residue redissolved in 30 mL EtOAc and washed sequentially with 20 mL water and 20 mL brine. The combined aqueous layers were extracted with further EtOAc (2×30 mL). The organic layers were combined and passed through a hydrophobic frit, and the solvent was removed under reduced pressure. The sample was dissolved in DCM and purified by column chromatography using a silica cartridge (120 g) with an ethyl acetate-cyclohexane solvent system [3CV, 10-20%; 3CV, 20%; 5CV, 20-50%; 9CV, 50%]. The appropriate fractions were combined and the solvent removed in vacuo to afford the title compound in a 2:1 ratio of the 1-((2-(trimethylsilyl)ethoxy)methyl)-1H-imidazole-4-carbonitrile and 1-((2-(trimethylsilyl)ethoxy)methyl)-1H-imidazole-5-carbonitrile regioisomers, as a pale yellow oil (1.71 g, 7.66 mmol, 71%). LCMS (System B): t_{RET}=1.08 min; MH⁺ 224 (both regioisomers).

### Intermediate 16: 2-bromo-1-((2-(trimethylsilyl)ethoxy)methyl)-1H-imidazole-4-carbonitrile

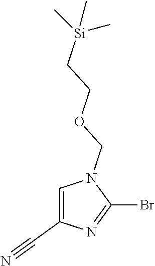 [SMILES: C[Si](C)(C)CCOCn1cc(C#N)nc1Br]

1-((2-(Trimethylsilyl)ethoxy)methyl)-1H-imidazole-4-carbonitrile (for an example preparation, see Intermediate 15, 1.68 g, 7.52 mmol (2:1 ratio of 1-((2-(trimethylsilyl)ethoxy)methyl)-1H-imidazole-4-carbonitrile and 1-((2-(trimethylsilyl)ethoxy)methyl)-1H-imidazole-5-carbonitrile)) was added to a round bottomed flask containing THF (40 mL). Once dissolved, N-bromosuccinimide (1.473 g, 8.27 mmol) was added, and the flask placed under an atmosphere of nitrogen. The reaction mixture was stirred at 60° C. overnight. Further 0.2 equivalents of N-bromosuccinimide (0.268 g, 1.504 mmol) was added to the reaction mixture and the reaction left stirring at 60° C. for a further 8 h. The reaction mixture was quenched with saturated sodium hydrogencarbonate solution (40 mL) and brine (40 mL) and extracted with EtOAc (3×40 mL). The combined organic layers were passed through a hydrophobic frit and the solvent removed under reduced pressure. The sample was loaded as a neat liquid and purified by column chromatography using a silica cartridge (80 g) with an ethyl acetate-cyclohexane solvent system [3CV, 0%; 7CV, 0-10%; 3CV, 10%]. The appropriate fractions were combined and the solvent removed in vacuo to give the crude product. The crude product was dissolved in diethyl ether and filtered through Celite®, the solvent was removed from the filtrate under reduced pressure to afford the title compound as a pale yellow oil, (1.24 g, 4.10 mmol, 55%). LCMS (System A): t_{RET}=1.23 min; MH⁺ 302, 304.

### Intermediate 17: 2-(1,5-dimethyl-6-oxo-1,6-dihydropyridin-3-yl)-1-((2-(trimethylsilyl)ethoxy)methyl)-1H-imidazole-4-carbonitrile

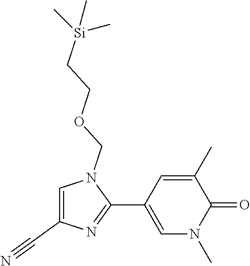 [SMILES: Cc1cc(-c2nc(C#N)cn2COCC[Si](C)(C)C)cn(C)c1=O]

1,3-Dimethyl-5-(4,4,5,5-tetramethyl-1,3,2-dioxaborolan-2-yl)pyridin-2(1H)-one (1.385 g, 5.56 mmol), 2-bromo-1-((2-(trimethylsilyl)ethoxy)methyl)-1H-imidazole-4-carbonitrile (for an example preparation, see Intermediate 16, 1.12 g, 3.71 mmol), and potassium carbonate (1.536 g, 11.12 mmol) were added to a microwave vial. 1,4-Dioxane (15 mL) and water (5 mL) were added to the vial, which was purged with nitrogen for 5 min prior to the addition of tetrakis(triphenylphosphine)palladium(0) (0.128 g, 0.111 mmol). After a further 5 min purge with nitrogen, the vial was capped and heated in the microwave at 110° C. for 1 h. The solvent was removed by evaporation under reduced pressure. The residue was redissolved in ethyl acetate and filtered through Celite®, the solvent again removed under reduced pressure. The sample was loaded in DCM and purified by column chromatography using a silica cartridge (80 g) with an ethyl acetate-cyclohexane solvent system [0-50%, 10CV; 50%, 15CV; 50-100%, 10CV; 100%, 5CV]. The appropriate fractions were combined and the solvent removed in vacuo to afford the title compound as a white solid, (933 mg, 2.71 mmol, 73%). LCMS (System A): t_{RET}=1.11 min; MH⁺ 345.

### Intermediate 18: 2-(1,5-dimethyl-6-oxo-1,6-dihydropyridin-3-yl)-1H-imidazole-4-carbonitrile

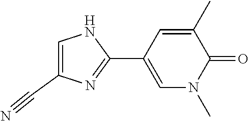 [SMILES: Cc1cc(-c2nc(C#N)c[nH]2)cn(C)c1=O]

2-(1,5-Dimethyl-6-oxo-1,6-dihydropyridin-3-yl)-1-((2-(trimethylsilyl)ethoxy)methyl)-1H-imidazole-4-carbonitrile (for an example preparation, see Intermediate 17, 933 mg, 2.71 mmol) was placed in a vial to which a solution of tetrabutylammonium fluoride in THF (1 M, 12 ml, 12.00 mmol) was added dropwise, the reaction vessel placed under an atmosphere of nitrogen by evacuation-refill. The reaction was stirred at 45° C. overnight, and the solvent removed under reduced pressure. The residue was redissolved in EtOAc (10 mL) and washed with brine solution (10 mL). The aqueous layer was extracted with further EtOAc (2×10 mL). The combined organic layers were passed through a hydrophobic frit and the solvent removed under reduced pressure. The crude product was redissolved in EtOAc and filtered through Celite®, washing with further EtOAc. The solvent was removed under reduced pressure to afford the crude product (908 mg) with ˜80% wt tetrabutylammonium fluoride contaminant. LCMS (System B): t_{RET}=0.49 min; MH⁺ 215.

### Intermediate 19: 1-benzyl-4-chloro-1H-imidazole

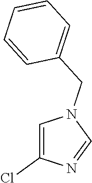 [SMILES: Clc1cn(Cc2ccccc2)cn1]

4-Chloro-1H-imidazole (100 mg, 0.975 mmol) was dissolved in DMF (2 mL) in a vial in an ice bath, and purged with nitrogen for 10 min. With the solution at 0° C., sodium hydride (60% dispersion in mineral oil) (50.7 mg, 1.268 mmol) was added and the solution stirred for 30 min. A solution of benzyl bromide (0.139 mL, 1.170 mmol) in DMF (2 mL) was added dropwise. Once all the reactant was added, the reaction mixture was allowed to warm to RT and stirred overnight under an atmosphere of nitrogen. The mixture was quenched with MeOH (0.5 mL) and the solvent removed under a stream of nitrogen. The samples were dissolved in 1:1 MeOH:DMSO (3 mL) and purified by MDAP (Method C). The solvent was removed in vacuo to afford the title compound as the minor regioisomer, an off-white solid (38 mg, 0.20 mmol, 20%). LCMS (System B): t_{RET}=0.92 min; MH⁺ 193, 195.

### Intermediate 20: 1-benzyl-2,4-dibromo-5-chloro-1H-imidazole

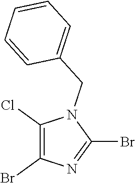 [SMILES: Clc1c(Br)nc(Br)n1Cc1ccccc1]

1-Benzyl-5-chloro-1H-imidazole (for an example preparation, see Intermediate 19, 38 mg, 0.197 mmol) was added to a round bottomed flask containing THF (1 mL). Once dissolved, N-bromosuccinimide (45.6 mg, 0.256 mmol) was added, and the flask placed under an atmosphere of nitrogen. The reaction mixture was heated to 60° C. and stirred overnight. The solvent was removed from the reaction mixture under reduced pressure, and the residue was dissolved in 1:1 MeOH:DMSO 0.9 mL and purified by MDAP (Method B). The solvent was evaporated in vacuo to afford the title compound as a white solid (13.8 mg, 0.04 mmol, 20%). LCMS (System B): t_{RET}=1.25 min; MH⁺ 349, 351, 353.

### Intermediate 21: 2-(1,5-dimethyl-6-oxo-1,6-dihydropyridin-3-yl)-1H-imidazole-4-carboxamide

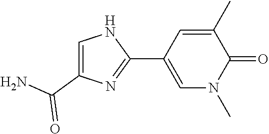 [SMILES: Cc1cc(-c2nc(C(N)=O)c[nH]2)cn(C)c1=O]

2-(1,5-Dimethyl-6-oxo-1,6-dihydropyridin-3-yl)-1-((2-(trimethylsilyl)ethoxy)methyl)-1H-imidazole-4-carbonitrile (for an example preparation, see Intermediate 17, 848 mg, 2.462 mmol) was added to a round bottomed flask and dissolved in anhydrous methanol (10 mL). The flask was purged with nitrogen by evacuation-refill, and trimethylsilylchloride (6.29 mL, 49.2 mmol) was added to the reaction mixture. The reaction mixture was stirred at 40° C. overnight under an atmosphere of nitrogen. The solvent was removed in vacuo and twice the residue was redissolved in methanol (30 mL) and solvent evaporated. The crude product was loaded in methanol and purified by SPE on a sulphonic acid (SCX) cartridge (20 g) with sequential solvent elution of methanol and 2M ammonia in methanol. Product containing fractions were combined and the solvent was removed under reduced pressure. The sample was dissolved in MeOH:DMSO (3 mL) and filtered, the filtrate purified by MDAP (Method B). The particulate residue was dissolved in dilute HCl (6 mL) and purified by MDAP (Method A). Appropriate fractions from the purification were combined and the solvent removed under a stream of nitrogen to afford the title compound as a white solid (290 mg, 1.25 mmol, 51%). LCMS (System B): t_{RET}=0.45 min; MH⁺ 233.

### Intermediate 22: 1-benzyl-4-fluoro-1H-imidazole

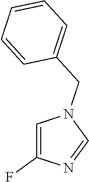 [SMILES: Fc1cn(Cc2ccccc2)cn1]

To a stirred solution of 4-fluoro-1H-imidazole (71 mg, 0.825 mmol) in DMF (3 mL) at 0° C. was added sodium hydride (60% dispersion in mineral oil) (49.5 mg, 1.237 mmol). The reaction mixture was stirred for 30 min, then benzyl bromide (0.147 mL, 1.237 mmol) was added. The reaction was allowed to warm to RT and stirred for a further 16 h. The solvent was removed in vacuo and the crude residue dissolved in 1:1 DMSO:MeOH (1.8 mL) and purified by MDAP (Method A) to afford the title compound as a colourless oil (71.6 mg, 0.386 mmol, 47%). LCMS (System A): t_{RET}=0.83 min; MH⁺ 177.

### Intermediate 23: 1-benzyl-2,4-dibromo-1H-imidazole

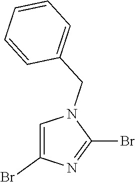 [SMILES: Brc1cn(Cc2ccccc2)c(Br)n1]

To a stirred solution of 2,4-dibromo-1H-imidazole (1.00 g, 4.43 mmol) in DMF (15 mL) at 0° C. was added sodium hydride (60% dispersion in mineral oil) (266 mg, 6.64 mmol). The reaction was stirred for 30 min, then benzyl bromide (0.789 mL, 6.64 mmol) was added. The reaction was allowed to warm to RT and stirred for a further 16 h. The solvent was removed in vacuo and the crude residue redissolved in MeOH (6 mL) and purified by MDAP (Method B) to afford the title compound as a light brown oil (1.16 g, 3.30 mmol, 75%). LCMS (System B): t_{RET}=1.10 min; MH⁺ 315, 317, 319.

### Intermediate 24: methyl 2-(3-(bromomethyl)phenyl)acetate

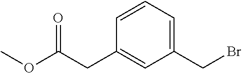

2-(3-(Bromomethyl)phenyl)acetic acid (10 g, 43.7 mmol) was added to methanol (35 mL) and toluene (70 mL) and the solution cooled to 0° C. Thionyl chloride (2.87 mL, 39.3 mmol) was added dropwise, and the reaction mixture stirred at 0° C. for 3 h. A solution of sodium hydrogencarbonate (3M, 90 mL) was added to toluene (45 mL), and this mixture cooled to 0° C. prior to addition to the reaction mixture. The organic layer was separated and washed with water (2×50 mL), then dried over sodium sulfate and filtered. The solvent was removed from the filtrate under reduced pressure to afford the title compound as a pale yellow liquid (7.6 g, 31.3 mmol, 72%).

### Intermediate 25: methyl 2-(3-((2-(1,5-dimethyl-6-oxo-1,6-dihydropyridin-3-yl)-1H-imidazol-1-yl)methyl)phenyl)acetate

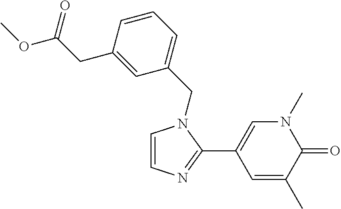 [SMILES: COC(=O)Cc1cccc(Cn2ccnc2-c2cc(C)c(=O)n(C)c2)c1]

To a mixture of 5-(1H-imidazol-2-yl)-1,3-dimethylpyridin-2(1H)-one (for an example preparation, see Intermediate 4, 3.0 g, 15.17 mmol) and methyl 2-(3-(bromomethyl)phenyl)acetate (for an example preparation, see Intermediate 24, 4.80 g, 19.73 mmol) in DMF (30 mL) was added potassium carbonate (4.19 g, 30.3 mmol). The resulting reaction mixture was stirred at RT for 15 h, after which the solvent was removed under reduced pressure. The residue was redissolved in 10% MeOH in DCM (250 mL) and water (250 mL), and the organic layer was separated and washed sequentially with water (3×150 mL) and brine (150 mL). The organic layer was then dried over sodium sulfate, filtered, and the filtrate concentrated under reduced pressure to give the crude product. The crude sample was purified by flash column chromatography using an elution gradient of 0-15% MeOH in DCM (12 g silica cartridge). The appropriate fractions were combined and the solvent removed in vacuo to afford the title compound as a brown gum (3.05 g, 8.68 mmol, 57%). LCMS (System E): t_{RET}=2.55 min; MH⁺ 352.

### Intermediate 26: 1-benzyl-5-bromo-4-chloro-1H-imidazole

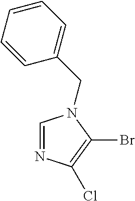 [SMILES: Clc1ncn(Cc2ccccc2)c1Br]

1-Benzyl-4-chloro-1H-imidazole (for an example preparation, see Intermediate 19, 165 mg, 0.856 mmol) was added to a round bottomed flask containing THF (4 mL) and a stirrer bar. Once dissolved, NBS (198 mg, 1.113 mmol) was added, the flask placed under an atmosphere of nitrogen. The reaction mixture was heated to 60° C. and left overnight, with stirring. The solvent was removed from the reaction mixture under reduced pressure, and the residue was dissolved in 1:1 MeOH:DMSO (3.6 mL) and purified by MDAP (Method A). The solvent was evaporated in vacuo to give the title compound as a white solid (12.7 mg). LCMS (System B): t_{RET}=1.07 min; MH⁺ 271, 273, 275.

### Example Preparation

### Example 1: 5-(1-benzyl-1H-imidazol-2-yl)-1,3-dimethylpyridin-2(1H)-one

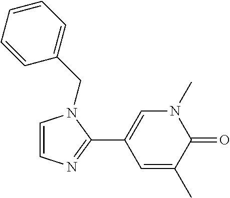 [SMILES: Cc1cc(-c2nccn2Cc2ccccc2)cn(C)c1=O]

To a stirred solution of 5-(1H-imidazol-2-yl)-1,3-dimethylpyridin-2(1H)-one (for an example preparation, see Intermediate 4, 300 mg, 1.586 mmol) in DMF (16 mL) at 0° C. was added sodium hydride (60% dispersion in mineral oil) (95 mg, 2.378 mmol). The reaction was stirred for 30 min, then benzyl bromide (0.283 mL, 2.378 mmol) was added. The reaction was allowed to warm to RT and stirred for a further 90 min. The solvent was removed in vacuo, and the crude residue was redissolved in EtOAc and filtered through a pad of Celite® (washing with 3×20 mL EtOAc). The solvent was removed in vacuo to afford a crude mixture. The crude residue was redissolved in DCM (5 mL) and loaded onto a 40 g silica column, and purified by flash column chromatography using a gradient of EtOAc to 30% EtOH in EtOAc over 20 CV to afford the title compound (311 mg, 1.113 mmol, 70.2%) as a light brown gum. LCMS (System B): t_{RET}=0.81 min; MH⁺ 280.

### Example 2: 1,3-dimethyl-5-(1-(pyridin-3-ylmethyl)-1H-imidazol-2-yl)pyridin-2(1H)-one

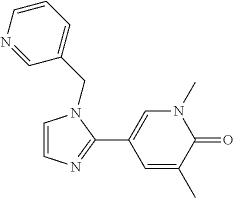 [SMILES: Cc1cc(-c2nccn2Cc2cccnc2)cn(C)c1=O]

3-((2-Bromo-1H-imidazol-1-yl)methyl)pyridine (for an example preparation, see Intermediate 1, 49.5 mg, 0.208 mmol), 1,3-dimethyl-5-(4,4,5,5-tetramethyl-1,3,2-dioxaborolan-2-yl)pyridin-2(1H)-one (51.8 mg, 0.208 mmol), potassium carbonate (144 mg, 1.040 mmol) and bis(triphenylphosphine)palladium(II) chloride (14.74 mg, 0.021 mmol) were added to a microwave vial along with ethanol (2 mL) and toluene (2 mL). The mixture was heated in a microwave at 120° C. for 1 h. The cooled mixture was then diluted in ethyl acetate (25 mL) and filtered. The solvents were then removed in vacuo. The sample was then loaded onto a 10 g silica cartridge and was purified by chromatography using the following method: 100% ethyl acetate for 2 CVs with a gradient run from 0%-25% ethanol in ethyl acetate run over 15 CVs 25% ethanol in ethyl acetate for 3 CV. Mixed fractions were obtained and combined, and the solvent was removed in vacuo The residue was then dissolved in DMSO (0.8 mL) and methanol (0.8 mL). The resulting mixture was split into two equal portions and purified by MDAP (Method B). Appropriate fractions were then combined and solvents removed in vacuo to give the title compound (3.3 mg, 0.012 mmol, 5.7%) which appeared as a yellow oil. LCMS (System B): t_{RET}=0.54 min; MH⁺ 281.

### Example 3: 1,3-dimethyl-5-(1-(pyridin-4-ylmethyl)-1H-imidazol-2-yl)pyridin-2(1H)-one

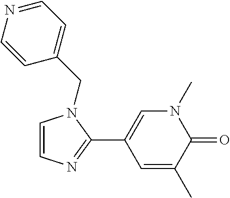 [SMILES: Cc1cc(-c2nccn2Cc2ccncc2)cn(C)c1=O]

4-((2-Bromo-1H-imidazol-1-yl)methyl)pyridine (for an example preparation, see Intermediate 2, 50 mg, 0.210 mmol), 1,3-dimethyl-5-(4,4,5,5-tetramethyl-1,3,2-dioxaborolan-2-yl)pyridin-2(1H)-one (commercially available from, for example, Milestone PharmaTech, 52.3 mg, 0.210 mmol), bis(triphenylphosphine)palladium(II) chloride (14.89 mg, 0.021 mmol) and potassium carbonate (145 mg, 1.050 mmol) were added to a microwave vial along with ethanol (2.0 mL) and toluene (2 mL). The mixture was heated in a microwave at 120° C. for 1 h. The cooled mixture was then diluted with ethyl acetate (25 mL) and filtered. The solvents were then removed in vacuo. The residue was then dissolved in DMSO (0.8 mL) and methanol (0.8 mL), then split into two equal portions and purified by 2×MDAP (Method B). Appropriate fractions were then combined and solvents removed in vacuo. The residue was determined to be the title compound (9.1 mg, 0.032 mmol, 15%) and appeared as a yellow solid. LCMS (System B): t_{RET}=0.40 min; MH⁺ 281.

### Example 4: 1,3-dimethyl-5-(1-(pyridin-2-ylmethyl)-1H-imidazol-2-yl)pyridin-2(1H)-one

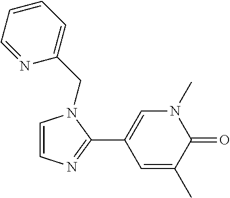 [SMILES: Cc1cc(-c2nccn2Cc2ccccn2)cn(C)c1=O]

1,3-Dimethyl-5-(4,4,5,5-tetramethyl-1,3,2-dioxaborolan-2-yl)pyridin-2(1H)-one (120 mg, 0.481 mmol), 2-((2-bromo-1H-imidazol-1-yl)methyl)pyridine (for an example preparation, see Intermediate 3, 76.3 mg, 0.320 mmol), and potassium carbonate (111 mg, 0.801 mmol) were added to a 5 mL microwave vial containing a stirrer bar. 1,4-Dioxane (1.5 mL) and water (0.5 mL) were added to the vial, which was purged with nitrogen for 5 min prior to the addition of tetrakis(triphenylphosphine)palladium(0) (11.11 mg, 9.61 μmol). After a further 5 minute purge with nitrogen, the vial was capped and heated in a microwave at 110° C. for 1 h. The solvents were removed by evaporation under reduced pressure. The crude product was dissolved in ethyl acetate and filtered through Celite®. The ethyl acetate was removed under reduced pressure and the sample was dissolved in 1:1 MeOH:DMSO (3 mL) and purified by MDAP (Method B). The solvent was evaporated in vacuo. NMR showed significant impurities. The samples were dissolved in 1:1 MeOH:DMSO (1 mL) and purified by MDAP (Method A). The solvent was evaporated in vacuo. The sample was dissolved in 1:1 MeOH:DMSO (1 mL) and purified by MDAP (Method B). The solvent was evaporated in vacuo to give the title compound as a white solid (10.5 mg). LCMS (System B): t_{RET}=0.62 min; MH⁺ 281.

### Example 5: 1,3-dimethyl-5-(1-(quinolin-8-ylmethyl)-1H-imidazol-2-yl)pyridin-2(1H)-one

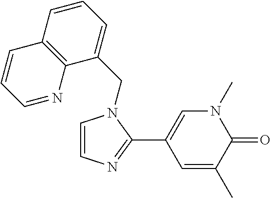 [SMILES: Cc1cc(-c2nccn2Cc2cccc3cccnc23)cn(C)c1=O]

5-(1H-Imidazol-2-yl)-1,3-dimethylpyridin-2(1H)-one (for an example preparation, see Intermediate 4, 0.312 g, 1.65 mmol) was dissolved in DMF (6.6 mL). 0.6 mL of the solution (0.15 mmol) was added to 8-(bromomethyl)quinoline (0.044 g, 0.2 mmol). Potassium carbonate (0.041 g, 0.300 mmol) and DMSO (0.2 mL) was added. The reaction vessel was sealed and heated in microwave using initial 600 W to 90° C. for 30 min. After cooling the reaction to rt, the samples in the reaction solvent (DMF, DMSO) were purified by MDAP (Method B). The solvent was dried under a stream of nitrogen give the title compound (5.3 mg, 0.016 mmol, 10%). LCMS (System A): t_{RET}=0.52 min; MH⁺ 331.

### Example 6: 4-((2-(1,5-dimethyl-6-oxo-1,6-dihydropyridin-3-yl)-1H-imidazol-1-yl)methyl)benzonitrile

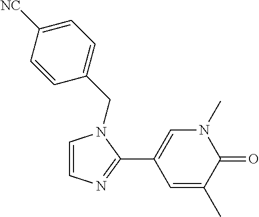 [SMILES: Cc1cc(-c2nccn2Cc2ccc(C#N)cc2)cn(C)c1=O]

5-(1H-Imidazol-2-yl)-1,3-dimethylpyridin-2(1H)-one (for an example preparation, see Intermediate 4, 0.312 g, 1.65 mmol) was dissolved in DMF (6.6 mL). 0.6 mL of the solution (0.15 mmol) was added to to 4-(bromomethyl)benzonitrile (0.2 mmol). Potassium carbonate (0.041 g, 0.300 mmol) and DMSO (0.2 mL) was added. The reaction vessel was sealed and heated in microwave using initial 600 W to 90° C. for 30 min. After cooling the reaction to RT, the sample in the reaction solvent (DMF, DMSO) was purified by MDAP (Method). The solvent was dried under a stream of nitrogen to give impure product. The sample were dissolved in DMSO (0.8 mL) and purified by MDAP (Method B). The solvent was dried under a stream of nitrogen to give the title compound (2.8 mg, 6%). LCMS (System A): t_{RET}=0.42 min; MH⁺ 305.

### Example 7: 5-((2-(1,5-dimethyl-6-oxo-1,6-dihydropyridin-3-yl)-1H-imidazol-1-yl)methyl)picolinonitrile

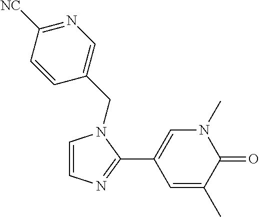 [SMILES: Cc1cc(-c2nccn2Cc2ccc(C#N)nc2)cn(C)c1=O]

5-(1H-Imidazol-2-yl)-1,3-dimethylpyridin-2(1H)-one (for an example preparation, see Intermediate 4, 0.312 g, 1.65 mmol) was dissolved in DMF (6.6 mL). 0.6 mL of the solution (0.15 mmol) was added to to 5-(bromomethyl)picolinonitrile (0.2 mmol). Potassium carbonate (0.041 g, 0.300 mmol) and dimethyl sulfoxide (DMSO) (0.2 mL) was added. The reaction vessel was sealed and heated in microwave using initial 600 W to 90° C. for 30 min. After cooling the reaction to rt, the sample in the reaction solvent (DMF, DMSO) was purified by MDAP (Method B). The solvent was dried under a stream of nitrogen to give impure product. The sample were dissolved in DMSO (0.8 mL) and purified by MDAP (Method B). The solvent was dried under a stream of nitrogen to give the title compound (2 mg, 4%). LCMS (System A): t_{RET}=0.35 min; MH⁺ 306.

### Example 8: 5-(1-(3-methoxybenzyl)-1H-imidazol-2-yl)-1,3-dimethylpyridin-2(1H)-one

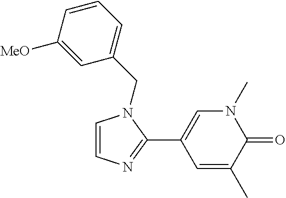 [SMILES: COc1cccc(Cn2ccnc2-c2cc(C)c(=O)n(C)c2)c1]

sodium hydride (60% dispersion in mineral oil) (0.053 g, 1.32 mmol) and 5-(1H-imidazol-2-yl)-1,3-dimethylpyridin-2(1H)-one (for an example preparation, see Intermediate 4, 0.114 g, 0.6 mmol) was dissolved in DMF (2.4 mL) and the mixture stirred at 22° C. for 15 min. 0.6 mL of the mixture (0.15 mmol core, 0.33 mmol sodium hydride) was then added to 1-(bromomethyl)-3-methoxybenzene (0.2 mmol). The reaction vessel was sealed and left stirring at 22° C. for 18 h. The reaction was quenched with 0.3 mL MeOH. The samples in DMF/MeOH were purified by MDAP (Method B). The solvent was dried under a stream of nitrogen to give the title compound (6.9 mg, 13%). LCMS (System A): t_{RET}=0.45 min; MH⁺ 310.

### Example 9: 1,3-dimethyl-5-(1-(4-(methylsulfonyl)benzyl)-1H-imidazol-2-yl)pyridin-2(1H)-one

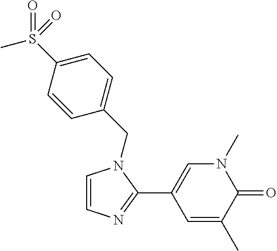 [SMILES: Cc1cc(-c2nccn2Cc2ccc(S(C)(=O)=O)cc2)cn(C)c1=O]

60% Sodium hydride (80 mg, 2 mmol) and 5-(1H-imidazol-2-yl)-1,3-dimethylpyridin-2(1H)-one (for an example preparation, see Intermediate 4, 0.284 g, 1.5 mmol) were dissolved in DMF (6 mL) and the mixture stirred at 22° C. for 15 min. 0.6 mL of the mixture was then added to 1-(bromomethyl)-4-(methylsulfonyl)benzene (0.15 mmol). The reaction vessels were sealed and left stirring at 22° C. for 18 hr. After 18 h further equivalent of sodium hydride (60% dispersion in mineral oil) (0.008 g, 0.200 mmol) was added to the reaction and the reaction was left stirring for 2 h at 22° C. The reaction was quenched with 0.3 mL MeOH. The sample was purified by MDAP (Method B). The solvent was removed under a stream of nitrogen to give the product (3.5 mg, 9.8 μmol, 6%). LCMS (System B): t_{RET}=0.64 min; MH⁺ 358.

### Example 10: Methyl 1-benzyl-2-(1,5-dimethyl-6-oxo-1,6-dihydropyridin-3-yl)-1H-imidazole-4-carboxylate

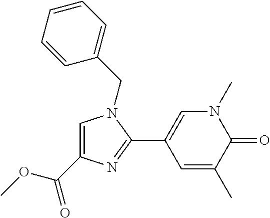 [SMILES: COC(=O)c1cn(Cc2ccccc2)c(-c2cc(C)c(=O)n(C)c2)n1]

Methyl 2-(1,5-dimethyl-6-oxo-1,6-dihydropyridin-3-yl)-1H-imidazole-4-carboxylate (for an example preparation, see Intermediate 8, 778 mg, 3.15 mmol) was dissolved in DMF (10 mL) in a round bottomed flask and placed in an ice bath. With the solution at 0° C., sodium hydride (60% dispersion in mineral oil) (164 mg, 4.09 mmol) was added and the solution stirred for 30 min. A solution of benzyl bromide (0.449 mL, 3.78 mmol) in DMF (10 mL) was added dropwise to the reaction mixture. The vessel was allowed to warm to RT and the reaction mixture stirred overnight. The mixture was quenched with MeOH (0.5 mL) and the solvent evaporated under reduced pressure. The crude product was dissolved in 30 mL EtOAc and washed with 20 mL water; the organic layer passed through a hydrophobic frit and the solvent removed under reduced pressure. The sample was loaded in DCM and purified by gradient elution column chromatography using an 80 g silica (Si) catridge with a 3:1 ethyl acetate:ethanol-cyclohexane solvent system [30-60%, 8CV; 60%, 12CV]. The appropriate fractions were combined and the solvent removed in vacuo. The main product batch was dissolved in 30 mL EtOAc and washed with brine solution (3×10 mL) and dilute HCl (0.2 M, 10 mL). The solvent was removed from the organic layer in vacuo to yield the title compound as a white solid (450 mg, 1.33 mmol, 42%). LCMS (System B): t_{RET}=0.83 min; MH⁺ 338. A second batch was obtained by dissolving less pure product fractions in 3 mL 1:1 MeOH:DMSO and purified by MDAP (Method B). The solvent was evaporated in vacuo to give the title compound as a colourless oil (164 mg, 0.49 mmol, 15%). LCMS (System B): t_{RET}=0.82 min; MH⁺ 338.

### Example 11: Methyl 1-benzyl-2-(1,5-dimethyl-6-oxo-1,6-dihydropyridin-3-yl)-1H-imidazole-5-carboxylate

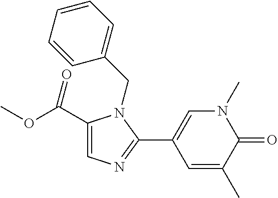 [SMILES: COC(=O)c1cnc(-c2cc(C)c(=O)n(C)c2)n1Cc1ccccc1]

Methyl 2-(1,5-dimethyl-6-oxo-1,6-dihydropyridin-3-yl)-1H-imidazole-4-carboxylate (for an example preparation, see Intermediate 8, 778 mg, 3.15 mmol) was dissolved in DMF (10 mL) in a round bottomed flask and placed in an ice bath. With the solution at 0° C., sodium hydride (60% dispersion in mineral oil) (164 mg, 4.09 mmol) was added and the solution stirred for 30 min. A solution of benzyl bromide (0.449 mL, 3.78 mmol) in DMF (10 mL) was added dropwise to the reaction mixture. The vessel was allowed to warm to RT and the reaction mixture stirred overnight. The mixture was quenched with MeOH (0.5 mL) and the solvent evaporated under reduced pressure. The crude product was dissolved in 30 mL EtOAc and washed with 20 mL water; the organic layer passed through a hydrophobic frit and the solvent removed under reduced pressure. The sample was loaded in DCM and purified by gradient elution column chromatography using an 80 g silica (Si) catridge with a 3:1 ethyl acetate:ethanol-cyclohexane solvent system [30-60%, 8CV; 60%, 12CV]. The appropriate fractions were combined and the solvent removed in vacuo. The product dissolved in 3 mL 1:1 MeOH:DMSO and purified further by MDAP (Method B). The solvent was evaporated in vacuo to give the title compound as a colourless oil (80 mg, 0.24 mmol, 8%). LCMS (System B): t_{RET}=0.89 min; MH⁺ 338.

### Example 12: 5-(1-benzyl-4-chloro-1H-imidazol-2-yl)-1,3-dimethylpyridin-2(1H)-one

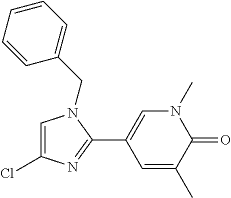 [SMILES: Cc1cc(-c2nc(Cl)cn2Cc2ccccc2)cn(C)c1=O]

5-(4-Chloro-1H-imidazol-2-yl)-1,3-dimethylpyridin-2(1H)-one (for an example preparation, see Intermediate 12, 74 mg, 0.331 mmol) was dissolved in DMF (1 mL) in a round bottomed flask and placed in an ice bath. With the solution at 0° C., sodium hydride (60% dispersion in mineral oil) (17.20 mg, 0.430 mmol) was added and the solution stirred for 30 min. (Bromomethyl)benzene (0.047 mL, 0.397 mmol) in DMF (1 mL) was added dropwise to the reaction mixture. The vessel was allowed to warm to RT and the reaction mixture stirred overnight. The reaction mixture was quenched with MeOH (0.5 mL) and the solvent removed under a stream of nitrogen. The samples were dissolved in 3 mL 1:1 MeOH:DMSO and purified by MDAP (Method B). The solvent was evaporated in vacuo and the desired fractions redissolved in 1 mL 1:1 MeOH:DMSO and purified further by MDAP (Method A) to yield the title compound as a pale oil, (38 mg, 0.12 mmol, 36%). LCMS (System A): t_{RET}=0.92 min; MH⁺ 314, 316.

### Example 13: 1,3-dimethyl-5-(1-(1-phenylethyl)-1H-imidazol-2-yl)pyridin-2(1H)-one

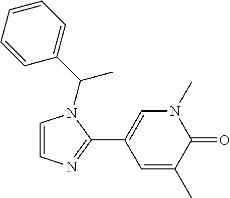 [SMILES: Cc1cc(-c2nccn2C(C)c2ccccc2)cn(C)c1=O]

To a stirred solution of 5-(1H-imidazol-2-yl)-1,3-dimethylpyridin-2(1H)-one (for an example preparation, see Intermediate 4, 50 mg, 0.264 mmol) in DMF (2 mL) at 0° C. was added sodium hydride (60% dispersion in mineral oil) (15.85 mg, 0.396 mmol). The reaction mixture was stirred at this temperature for 30 min prior to addition of (1-bromoethyl)benzene (0.054 mL, 0.396 mmol). The reaction was allowed to warm to RT and stirred for a further 3 h. Further sodium hydride (60% dispersion in mineral oil) (15.85 mg, 0.396 mmol) was added, and the reaction mixture stirred for 30 min, after which further (1-bromoethyl)benzene (0.054 mL, 0.396 mmol) was added and the reaction allowed to stir at RT for a further 2 h. The solvent was removed in vacuo, and the crude residue taken up in EtOAc (10 mL). The reaction mixture was filtered through Celite®, washed with EtOAc (3×10 mL), and the solvent removed in vacuo. The crude product was purified by MDAP (Method B) and the solvent removed in vacuo. The sample was dissolved in MeOH, and loaded onto a 1 g SCX cartridge, washing sequentially with MeOH (10 column volumes), and ammonia in MeOH (2M, 10 column volumes). The appropriate fractions were combined, and the solvent removed in vacuo to give the title compound as a colourless solid (29.7 mg, 0.10 mmol, 38%). LCMS (System A): t_{RET}=0.86 min; MH⁺ 294.

### Example 14: 1,3-dimethyl-5-(1-(pyrimidin-2-ylmethyl)-1H-imidazol-2-yl)pyridin-2(1H)-one

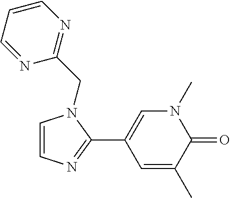 [SMILES: Cc1cc(-c2nccn2Cc2ncccn2)cn(C)c1=O]

To a stirred solution of 5-(1H-imidazol-2-yl)-1,3-dimethylpyridin-2(1H)-one (for an example preparation, see Intermediate 4, 50 mg, 0.264 mmol) in DMF (2 mL) at 0° C. was added sodium hydride (60% dispersion in mineral oil) (15.85 mg, 0.396 mmol). The reaction was stirred for 30 min, then 2-(chloromethyl)pyrimidine (0.041 mL, 0.396 mmol) was added. The reaction was allowed to warm to RT and stirred for a further 3 h. Further sodium hydride (60% dispersion in mineral oil) (15.85 mg, 0.396 mmol) was added, and the reaction stirred for 30 min, after which further 2-(chloromethyl)pyrimidine (0.041 mL, 0.396 mmol) was added and the reaction allowed to stir at RT for a further 62 h. The solvent was removed in vacuo, and the crude residue redissolved in EtOAc (10 mL) and filtered through a pad of Celite®, washing with EtOAc (3×10 mL). The solvent was removed in vacuo and the crude residue purified by MDAP (Method B) to give the title compound as a colourless solid (30.3 mg, 0.10 mmol, 39% yield). LCMS (System B): t_{RET}=0.53 min; MH⁺ 282.

### Example 15: 1,3-dimethyl-5-(1-(pyrimidin-5-ylmethyl)-1H-imidazol-2-yl)pyridin-2(1H)-one

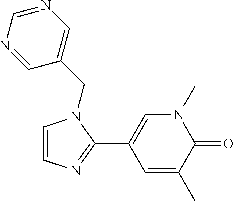 [SMILES: Cc1cc(-c2nccn2Cc2cncnc2)cn(C)c1=O]

To a stirred solution of 5-(1H-imidazol-2-yl)-1,3-dimethylpyridin-2(1H)-one (for an example preparation, see Intermediate 4, 50 mg, 0.264 mmol) in DMF (2 mL) at 0° C. was added sodium hydride (60% dispersion in mineral oil) (15.85 mg, 0.396 mmol). The reaction was stirred for 30 min, then 5-(chloromethyl)pyrimidine (51.0 mg, 0.396 mmol) was added. The reaction was allowed to warm to RT and stirred for a further 3 h. Further sodium hydride (60% dispersion in mineral oil) (15.85 mg, 0.396 mmol) was added, and the reaction stirred for 30 min, after which 5-(chloromethyl)pyrimidine (51.0 mg, 0.396 mmol) was added and the reaction allowed to stir at RT for a further 62 h. The solvent was removed in vacuo, and the crude residue redissolved in EtOAc (10 mL) and filtered through a pad of Celite®, washing with EtOAc (3×10 mL). The solvent was removed in vacuo and the crude residue purified by MDAP (Method B) to give the title compound as a colourless solid (16.1 mg, 0.05 mmol, 21%). LCMS (System B): t_{RET}=0.49 min; MH⁺ 282.

### Example 16: 1,3-dimethyl-5-(1-(3-(trifluoromethyl)benzyl)-1H-imidazol-2-yl)pyridin-2(1H)-one

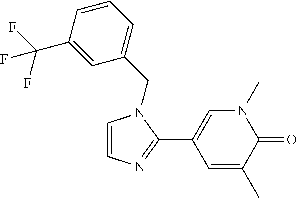 [SMILES: Cc1cc(-c2nccn2Cc2cccc(C(F)(F)F)c2)cn(C)c1=O]

A stock solution of 5-(1H-imidazol-2-yl)-1,3-dimethylpyridin-2(1H)-one (for an example preparation, see Intermediate 4, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 1-(bromomethyl)-3-(trifluoromethyl)benzene (0.15 mmol) which was sealed and left to stir for 3 h at RT. Methanol (50 uL) was added to the reaction mixture, and the sample loaded directly for purification by MDAP (Method B). The solvent was removed under a stream of nitrogen to give the title compound (29.1 mg, 0.08 mmol, 50%). LCMS (System A): t_{RET}=0.56 min; MH⁺ 348.

### Example 17: 5-(1-(2-chlorobenzyl)-1H-imidazol-2-yl)-1,3-dimethylpyridin-2(1H)-one

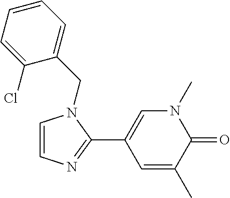 [SMILES: Cc1cc(-c2nccn2Cc2ccccc2Cl)cn(C)c1=O]

A stock solution of 5-(1H-imidazol-2-yl)-1,3-dimethylpyridin-2(1H)-one (for an example preparation, see Intermediate 7, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 1-(bromomethyl)-2-chlorobenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to give the title compound (21.8 mg, 0.07 mmol, 42%). LCMS (System A): t_{RET}=0.50 min; MH⁺ 314.

### Example 18: 1,3-dimethyl-5-(1-(4-methylbenzyl)-1H-imidazol-2-yl)pyridin-2(1H)-one

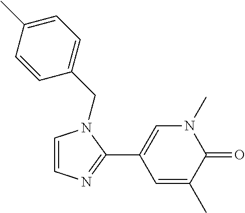 [SMILES: Cc1ccc(Cn2ccnc2-c2cc(C)c(=O)n(C)c2)cc1]

A stock solution of 5-(1H-imidazol-2-yl)-1,3-dimethylpyridin-2(1H)-one (for an example preparation, see Intermediate 4, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 1-(bromomethyl)-4-methylbenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to give the title compound (28.3 mg, 0.10 mmol, 58%). LCMS (System A): t_{RET}=0.51 min; MH⁺ 294.

### Example 19: 5-(1-(3,4-dichlorobenzyl)-1H-imidazol-2-yl)-1,3-dimethylpyridin-2(1H)-one

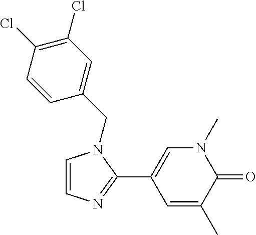 [SMILES: Cc1cc(-c2nccn2Cc2ccc(Cl)c(Cl)c2)cn(C)c1=O]

A stock solution of 5-(1H-imidazol-2-yl)-1,3-dimethylpyridin-2(1H)-one (for an example preparation, see Intermediate 4, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 4-(bromomethyl)-1,2-dichlorobenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to give the title compound (25.1 mg, 0.07 mmol, 43%). LCMS (System A): t_{RET}=0.60 min; MH⁺ 348, 350.

### Example 20: 5-(1-(4-methoxybenzyl)-1H-imidazol-2-yl)-1,3-dimethylpyridin-2(1H)-one

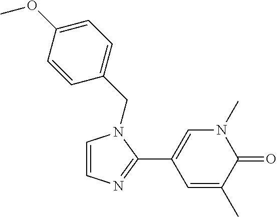 [SMILES: COc1ccc(Cn2ccnc2-c2cc(C)c(=O)n(C)c2)cc1]

A stock solution of 5-(1H-imidazol-2-yl)-1,3-dimethylpyridin-2(1H)-one (for an example preparation, see Intermediate 4, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 1-(bromomethyl)-4-methoxybenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to give the title compound (22.7 mg, 0.07 mmol, 44%). LCMS (System A): t_{RET}=0.46 min; MH⁺ 310.

### Example 21: 5-(1-(3,5-difluorobenzyl)-1H-imidazol-2-yl)-1,3-dimethylpyridin-2(1H)-one

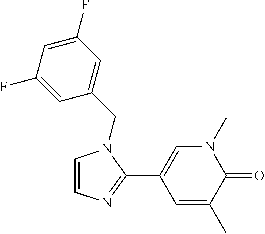 [SMILES: Cc1cc(-c2nccn2Cc2cc(F)cc(F)c2)cn(C)c1=O]

A stock solution of 5-(1H-imidazol-2-yl)-1,3-dimethylpyridin-2(1H)-one (for an example preparation, see Intermediate 4, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 1-(bromomethyl)-3,5-difluorobenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to give the title compound (23.4 mg, 0.07 mmol, 45%). LCMS (System A): t_{RET}=0.48 min; MH⁺ 316.

### Example 22: 1,3-dimethyl-5-(1-(4-(trifluoromethyl)benzyl)-1H-imidazol-2-yl)pyridin-2(1H)-one

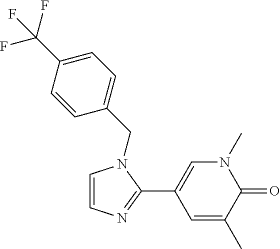 [SMILES: Cc1cc(-c2nccn2Cc2ccc(C(F)(F)F)cc2)cn(C)c1=O]

A stock solution of 5-(1H-imidazol-2-yl)-1,3-dimethylpyridin-2(1H)-one (for an example preparation, see Intermediate 4, 1.5 mmol, 284 mg), and sodium hydride (120 mg) was prepared in DMF (7.0 mL), and this was stirred for 15 min. 0.7 mL of the solution was then transferred to a 3 mL vial containing 1-(bromomethyl)-4-(trifluoromethyl)benzene (0.15 mmol) which was sealed and left to stir for 3 h at RT. Methanol (50 μL) was added to the reaction mixture, and the sample loaded directly for purification by CAT MDAP (Method B). The solvent was removed under a stream of nitrogen to give the title compound (22.8 mg, 0.07 mmol, 39%). LCMS (System A): t_{RET}=0.58 min; MH⁺ 348.

### Example 23: 5-(1-(2-methoxybenzyl)-1H-imidazol-2-yl)-1,3-dimethylpyridin-2(1H)-one

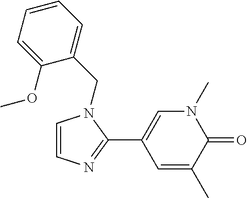 [SMILES: COc1ccccc1Cn1ccnc1-c1cc(C)c(=O)n(C)c1]

A stock solution of 5-(1H-imidazol-2-yl)-1,3-dimethylpyridin-2(1H)-one (for an example preparation, see Intermediate 4, 1.5 mmol, 284 mg), and sodium hydride (120 mg) was prepared in DMF (7.0 mL), and this was stirred for 15 min. 0.7 mL of the solution was then transferred to a 3 mL vial containing 1-(bromomethyl)-2-methoxybenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to give the title compound (27.7 mg, 0.09 mmol, 54%). LCMS (System A): t_{RET}=0.48 min; MH⁺ 310.

### Example 24: 5-(1-(3-chloro-5-fluoro-4-methoxybenzyl)-1H-imidazol-2-yl)-1,3-dimethylpyridin-2(1H)-one

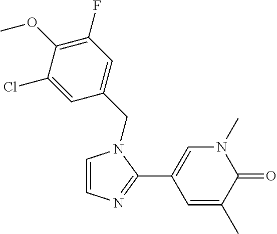 [SMILES: COc1c(F)cc(Cn2ccnc2-c2cc(C)c(=O)n(C)c2)cc1Cl]

A stock solution of 5-(1H-imidazol-2-yl)-1,3-dimethylpyridin-2(1H)-one (for an example preparation, see Intermediate 4, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 5-(bromomethyl)-1-chloro-3-fluoro-2-methoxybenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to give the title compound (28.3 mg, 0.08 mmol, 47%). LCMS (System A): t_{RET}=0.55 min; MH⁺ 362, 364.

### Example 25: 5-(1-(3-fluoro-4-methoxybenzyl)-1H-imidazol-2-yl)-1,3-dimethylpyridin-2(1H)-one

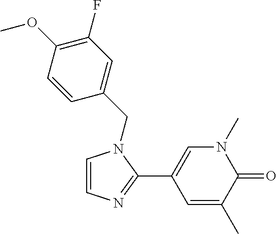 [SMILES: COc1ccc(Cn2ccnc2-c2cc(C)c(=O)n(C)c2)cc1F]

A stock solution of 5-(1H-imidazol-2-yl)-1,3-dimethylpyridin-2(1H)-one (for an example preparation, see Intermediate 4, 1.5 mmol, 284 mg), and sodium hydride (60% dispersion in mineral oil) (120 mg) was prepared in DMF (7.0 mL), and this was stirred for 15 min. 0.7 mL of the solution was then transferred to a 3 mL vial containing 4-(bromomethyl)-2-fluoro-1-methoxybenzene (0.15 mmol) which was sealed and left to stir for 3 h at RT. Methanol (50 μL) was added to the reaction mixture, and the sample loaded directly for purification by MDAP (Method B). The solvent was removed under a stream of nitrogen to afford the title compound (24.6 mg, 0.08 mmol, 45%). LCMS (System A): t_{RET}=0.46 min; MH⁺ 328.

### Example 26: (S)-5-(1-benzyl-4-(2-(hydroxymethyl)pyrrolidine-1-carbonyl)-1H-imidazol-2-yl)-1,3-dimethylpyridin-2(1H)-one

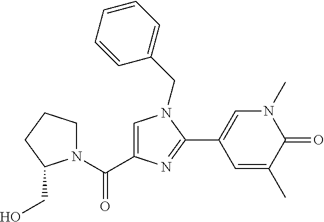 [SMILES: Cc1cc(-c2nc(C(=O)N3CCC[C@H]3CO)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 18 mg, 0.055 mmol) was added to a vial containing a stirrer bar, and dissolved in DMF (1 mL). HATU (22.86 mg, 0.060 mmol) was added, and the reaction mixture stirred under an atmosphere of nitrogen at RT for 1 h. A solution of (S)-pyrrolidin-2-ylmethanol (6.08 mg, 0.060 mmol) and DIPEA (9.55 μl, 0.055 mmol) in DMF (0.5 mL) was added, and the reaction stirred for a further 1 h. The solvent was removed under a stream of nitrogen. The sample was dissolved in 1:1 MeOH:DMSO 0.9 mL and purified by MDAP (Method B). The solvent was evaporated in vacuo to give the title compound as a white solid (18.5 mg, 0.04 mmol, 74%). LCMS (System B): t_{RET}=0.77 min; MH⁺ 407.

### Example 27: 1,3-dimethyl-5-(1-((6-methylpyridin-2-yl)methyl)-1H-imidazol-2-yl)pyridin-2(1H)-one

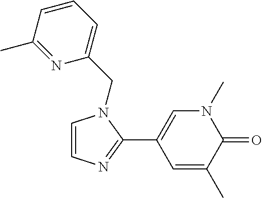 [SMILES: Cc1cccc(Cn2ccnc2-c2cc(C)c(=O)n(C)c2)n1]

To a stirred solution of 5-(1H-imidazol-2-yl)-1,3-dimethylpyridin-2(1H)-one (for an example preparation, see Intermediate 4, 50 mg, 0.264 mmol) in DMF (2 mL) at 0° C. was added sodium hydride (60% dispersion in mineral oil) (15.85 mg, 0.396 mmol). The reaction was stirred for 30 min, then 2-(bromomethyl)-6-methylpyridine (73.7 mg, 0.396 mmol) was added. The reaction was allowed to warm to RT and stirred for a further 18 h. The solvent was removed in vacuo and the crude residue redissolved in EtOAc (10 mL). The suspension was filtered through a pad of Celite®, washing with EtOAc (3×10 mL) and the solvent removed in vacuo. The compound was purified by MDAP (Method B) and the dried product redissolved in MeOH and loaded onto a 1 g SCX column. This was washed with 5 column volumes of MeOH, followed by 5 column volumes of 2M ammonia in MeOH. The appropriate fractions were combined and the solvent removed in vacuo to afford the title compound as a colourless solid (46 mg, 0.15 mmol, 58%). LCMS (System B): t_{RET}=0.69 min; MH⁺ 295.

### Example 28: 5-(1-(2-fluorobenzyl)-1H-imidazol-2-yl)-1,3-dimethylpyridin-2(1H)-one

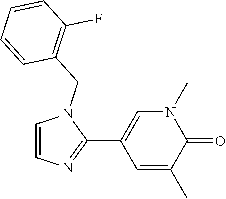 [SMILES: Cc1cc(-c2nccn2Cc2ccccc2F)cn(C)c1=O]

A stock solution of 5-(1H-imidazol-2-yl)-1,3-dimethylpyridin-2(1H)-one (for an example preparation, see Intermediate 4, 1.05 mmol, 199 mg), and sodium hydride (60% dispersion in mineral oil) (56 mg) was prepared in DMF (4.9 mL), and this was stirred for 15 min. 0.7 mL of the solution was then transferred to a 3 mL vial containing 1-(bromomethyl)-2-fluorobenzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen to afford the title compound (23 mg, 0.08 mmol, 46%). LCMS (System A): t_{RET}=0.44 min; MH⁺ 298.

### Example 29: 5-(1-(4-fluorobenzyl)-1H-imidazol-2-yl)-1,3-dimethylpyridin-2(1H)-one

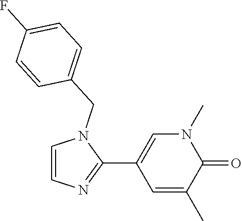 [SMILES: Cc1cc(-c2nccn2Cc2ccc(F)cc2)cn(C)c1=O]

A stock solution of 5-(1H-imidazol-2-yl)-1,3-dimethylpyridin-2(1H)-one (for an example preparation, see Intermediate 4, 1.05 mmol, 199 mg), and sodium hydride (60% dispersion in mineral oil) (56 mg) was prepared in DMF (4.9 mL), and this was stirred for 15 min. 0.7 mL of the solution was then transferred to a 3 mL vial containing 1-(bromomethyl)-4-fluorobenzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen to afford the title compound (22.7 mg, 0.08 mmol, 46%). LCMS (System A): t_{RET}=0.45 min; MH⁺ 298.

### Example 30: 1,3-dimethyl-5-(1-(4-(trifluoromethoxy)benzyl)-1H-imidazol-2-yl)pyridin-2(1H)-one

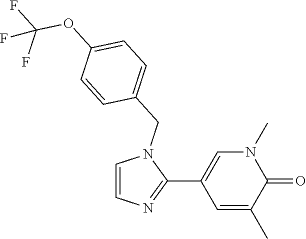 [SMILES: Cc1cc(-c2nccn2Cc2ccc(OC(F)(F)F)cc2)cn(C)c1=O]

A stock solution of 5-(1H-imidazol-2-yl)-1,3-dimethylpyridin-2(1H)-one (for an example preparation, see Intermediate 4, 1.05 mmol, 199 mg), and sodium hydride (60% dispersion in mineral oil) (56 mg) was prepared in DMF (4.9 mL), and this was stirred for 15 min. 0.7 mL of the solution was then transferred to a 3 mL vial containing 1-(bromomethyl)-4-(trifluoromethoxy)benzene (0.15 mmol) which was sealed and left to stir for 3 h at RT. Methanol (200 uL) was added to the reaction mixture, and the sample loaded directly for purification by MDAP (Method B). The solvent was removed under a stream of nitrogen to afford the title compound (32.4 mg, 0.09 mmol, 54%). LCMS (System A): t_{RET}=0.61 min; MH⁺ 364.

### Example 31: 5-(1-(4-(difluoromethoxy)benzyl)-1H-imidazol-2-yl)-1,3-dimethylpyridin-2(1H)-one

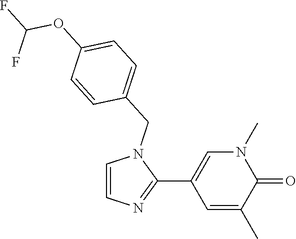 [SMILES: Cc1cc(-c2nccn2Cc2ccc(OC(F)F)cc2)cn(C)c1=O]

A stock solution of 5-(1H-imidazol-2-yl)-1,3-dimethylpyridin-2(1H)-one (for an example preparation, see Intermediate 4, 1.05 mmol, 199 mg), and sodium hydride (60% dispersion in mineral oil) (56 mg) was prepared in DMF (4.9 mL), and this was stirred for 15 min. 0.7 mL of the solution was then transferred to a 3 mL vial containing 1-(bromomethyl)-4-(difluoromethoxy)benzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen to afford the title compound (27.5 mg, 0.08 mmol, 48%). LCMS (System A): t_{RET}=0.51 min; MH⁺ 346.

### Example 32: 5-(1-(2,3-dichlorobenzyl)-1H-imidazol-2-yl)-1,3-dimethylpyridin-2(1H)-one

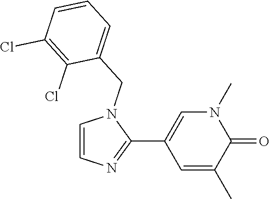 [SMILES: Cc1cc(-c2nccn2Cc2cccc(Cl)c2Cl)cn(C)c1=O]

A stock solution of 5-(1H-imidazol-2-yl)-1,3-dimethylpyridin-2(1H)-one (for an example preparation, see Intermediate 4, 1.05 mmol, 199 mg), and sodium hydride (60% dispersion in mineral oil) (56 mg) was prepared in DMF (4.9 mL), and this was stirred for 15 min. 0.7 mL of the solution was then transferred to a 3 mL vial containing 1-(bromomethyl)-2,3-dichlorobenzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen and the resulting sample dissolved in DMSO (1 mL) before repurification by MDAP (Method A). The solvent was removed under a stream of nitrogen to afford the title compound (16.3 mg, 0.05 mmol, 28%). LCMS (System A): t_{RET}=0.57 min; MH⁺ 348, 350.

### Example 33: 5-(1-(3-chlorobenzyl)-1H-imidazol-2-yl)-1,3-dimethylpyridin-2(1H)-one

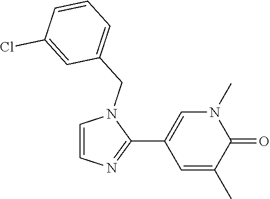 [SMILES: Cc1cc(-c2nccn2Cc2cccc(Cl)c2)cn(C)c1=O]

A stock solution of 5-(1H-imidazol-2-yl)-1,3-dimethylpyridin-2(1H)-one (for an example preparation, see Intermediate 4, 1.05 mmol, 199 mg), and sodium hydride (60% dispersion in mineral oil) (56 mg) was prepared in DMF (4.9 mL), and this was stirred for 15 min. 0.7 mL of the solution was then transferred to a 3 mL vial containing 1-(bromomethyl)-3-chlorobenzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen to afford the title compound (27.5 mg, 0.08 mmol, 48%). LCMS (System A): t_{RET}=0.51 min; MH⁺ 314, 316.

### Example 34: 1-benzyl-2-(1,5-dimethyl-6-oxo-1,6-dihydropyridin-3-yl)-1H-imidazole-4-carboxylic acid

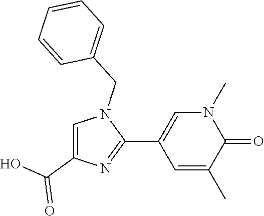 [SMILES: Cc1cc(-c2nc(C(=O)O)cn2Cc2ccccc2)cn(C)c1=O]

A sample of lithium 1-benzyl-2-(1,5-dimethyl-6-oxo-1,6-dihydropyridin-3-yl)-1H-imidazole-4-carboxylate (for an example preparation, see Intermediate 13, 10 mg, 0.03 mmol) was dissolved in 1:1 MeOH:DMSO (0.9 mL) and purified by MDAP (Method A). The solvent was evaporated in vacuo to afford the title compound as a colourless oil (5.4 mg, 0.02 mmol, 55%). LCMS (System A): t_{RET}=0.59 min; MH⁺ 324.

### Example 35: 1,3-dimethyl-5-(1-((5-methylpyridin-3-yl)methyl)-1H-imidazol-2-yl)pyridin-2(1H)-one

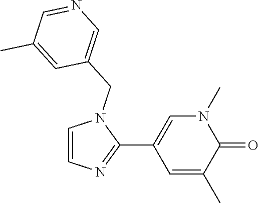 [SMILES: Cc1cncc(Cn2ccnc2-c2cc(C)c(=O)n(C)c2)c1]

To a stirred solution of 5-(1H-imidazol-2-yl)-1,3-dimethylpyridin-2(1H)-one (for an example preparation, see Intermediate 4, 50 mg, 0.264 mmol) in DMF (2 mL) at 0° C. was added sodium hydride (60% dispersion in mineral oil) (26.4 mg, 0.661 mmol). The reaction was stirred for 30 min, then 3-(chloromethyl)-5-methylpyridine hydrochloride salt (70.6 mg, 0.396 mmol) was added. The reaction was allowed to warm to RT and stirred for a further 18 h. The solvent was removed in vacuo and the crude residue redissolved in EtOAc (10 mL). The suspension was filtered through a pad of Celite®, washing with EtOAc (3×10 mL) and the solvent removed in vacuo. The compound was purified by MDAP (Method B) to afford the title compound as a light yellow solid (41 mg, 0.13 mmol, 50%). LCMS (System B): t_{RET}=0.63 min; MH⁺ 295.

### Example 36: 1,3-dimethyl-5-(1-(3-methylbenzyl)-1H-imidazol-2-yl)pyridin-2(1H)-one

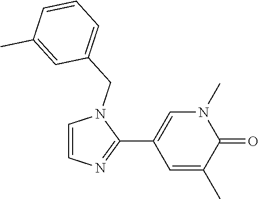 [SMILES: Cc1cccc(Cn2ccnc2-c2cc(C)c(=O)n(C)c2)c1]

A stock solution of 5-(1H-imidazol-2-yl)-1,3-dimethylpyridin-2(1H)-one (for an example preparation, see Intermediate 4, 0.45 mmol, 85 mg), and sodium hydride (60% dispersion in mineral oil) (24 mg) was prepared in DMF (2.1 mL), and this was stirred for 15 min. 0.7 mL of the solution was then transferred to a 3 mL vial containing 1-(bromomethyl)-3-methylbenzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen to afford the title compound (39.2 mg, 0.13 mmol, 80%). LCMS (System A): t_{RET}=0.50 min; MH⁺ 294.

### Example 37: 5-(1-(3-fluorobenzyl)-1H-imidazol-2-yl)-1,3-dimethylpyridin-2(1H)-one

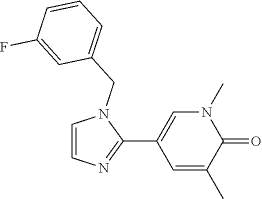 [SMILES: Cc1cc(-c2nccn2Cc2cccc(F)c2)cn(C)c1=O]

A stock solution of 5-(1H-imidazol-2-yl)-1,3-dimethylpyridin-2(1H)-one (for an example preparation, see Intermediate 4, 0.45 mmol, 85 mg), and sodium hydride (60% dispersion in mineral oil) (24 mg) was prepared in DMF (2.1 mL), and this was stirred for 15 min. 0.7 mL of the solution was then transferred to a 3 mL vial containing 1-(bromomethyl)-3-fluorobenzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen to afford the title compound (42.5 mg, 0.14 mmol, 86%). LCMS (System A): t_{RET}=0.45 min; MH⁺ 298.

### Example 38: 5-(1-(4-chlorobenzyl)-1H-imidazol-2-yl)-1,3-dimethylpyridin-2(1H)-one

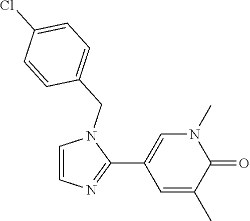 [SMILES: Cc1cc(-c2nccn2Cc2ccc(Cl)cc2)cn(C)c1=O]

A stock solution of 5-(1H-imidazol-2-yl)-1,3-dimethylpyridin-2(1H)-one (for an example preparation, see Intermediate 4, 0.45 mmol, 85 mg), and sodium hydride (60% dispersion in mineral oil) (24 mg) was prepared in DMF (2.1 mL), and this was stirred for 15 min. 0.7 mL of the solution was then transferred to a 3 mL vial containing 1-(bromomethyl)-4-chlorobenzene (0.15 mmol) which was sealed and left to stir for 3 h at RT. Methanol (200 μL) was added to the reaction mixture, and the sample loaded directly for purification by MDAP (Method B). The solvent was removed under a stream of nitrogen to afford the title compound (27.9 mg, 0.09 mmol, 53%). LCMS (System A): t_{RET}=0.52 min; MH⁺ 314.

### Example 39: 1,3-dimethyl-5-(1-(pyridazin-3-ylmethyl)-1H-imidazol-2-yl)pyridin-2(1H)-one

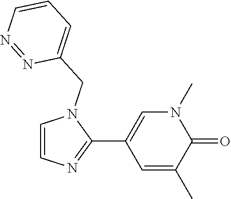 [SMILES: Cc1cc(-c2nccn2Cc2cccnn2)cn(C)c1=O]

To a stirred solution of 5-(1H-imidazol-2-yl)-1,3-dimethylpyridin-2(1H)-one (for an example preparation, see Intermediate 4, 50 mg, 0.264 mmol) in DMF (2 mL) at 0° C. was added sodium hydride (60% dispersion in mineral oil) (26.4 mg, 0.661 mmol). The reaction was stirred for 30 min, then 3-(bromomethyl)pyridazine hydrobromide salt (101 mg, 0.396 mmol) was added. The reaction was allowed to warm to RT and stirred for a further 3 h. The solvent was removed in vacuo and the crude residue redissolved in EtOAc (10 mL). The suspension was filtered through a pad of Celite®, washing with EtOAc (3×10 mL) and the solvent removed in vacuo. The compound was purified by MDAP (Method B) to afford the title compound as a light yellow solid (48 mg, 0.16 mmol, 61%). LCMS (System B): t_{RET}=0.48 min; MH⁺ 282.

### Example 40: 5-(1-benzyl-4-nitro-1H-imidazol-2-yl)-1,3-dimethylpyridin-2(1H)-one

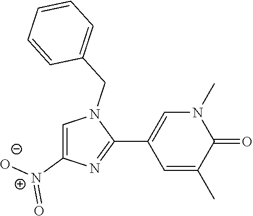 [SMILES: Cc1cc(-c2nc([N+](=O)[O-])cn2Cc2ccccc2)cn(C)c1=O]

A solution of 1,3-dimethyl-5-(4,4,5,5-tetramethyl-1,3,2-dioxaborolan-2-yl)pyridin-2(1H)-one (Available form Milestone PharmaTech, 550 mg, 2.207 mmol), 1-benzyl-2-bromo-4-nitro-1H-imidazole (for an example preparation, see Intermediate 14, 415 mg, 1.471 mmol), tetrakis(triphenylphosphine)palladium(0) (85 mg, 0.074 mmol) and potassium carbonate (610 mg, 4.41 mmol) in 1,4-dioxane (8 mL) and water (2 mL) was heated to 110° C. for 90 min. The solvent was removed in vacuo and the crude residue suspended in EtOAc (10 mL) and filtered through Celite®, washing with EtOAc (3×10 mL) and the solvent removed in vacuo. Purification by MDAP (Method B) afforded the title compound as a light yellow solid (344 mg, 1.01 mmol, 69%). LCMS (System B): t_{RET}=0.87 min; MH⁺ 325.

### Example 41: rac-5-(5-chloro-1-(1-phenylethyl)-1H-imidazol-2-yl)-1,3-dimethylpyridin-2(1H)-one

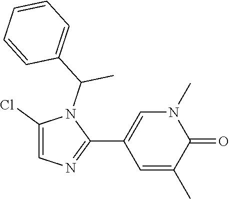 [SMILES: Cc1cc(-c2ncc(Cl)n2C(C)c2ccccc2)cn(C)c1=O]

To a stirred solution of 1,3-dimethyl-5-(1-(1-phenylethyl)-1H-imidazol-2-yl)pyridin-2(1H)-one (for an example preparation, see Example 13, 25 mg, 0.085 mmol) in chloroform (2 mL) was added 2-chloro-1,3-bis(methoxycarbonyl)guanidine (21.43 mg, 0.102 mmol), and the reaction mixture stirred for 18 h at RT. The solvent was removed in vacuo, and the reaction dissolved in MeOH (1.6 mL) and filtered through a syringe filter. Purification by MDAP (Method B) afforded the title compound as a colourless oil (9.4 mg, 0.027 mmol, 32%). LCMS (System B): t_{RET}=1.00 min; MH⁺ 328, 330.

### Example 42: 1-benzyl-2-(1,5-dimethyl-6-oxo-1,6-dihydropyridin-3-yl)-N,N-dimethyl-1H-imidazole-4-carboxamide

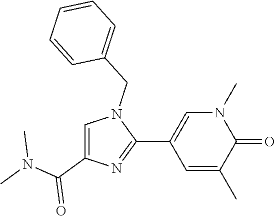 [SMILES: Cc1cc(-c2nc(C(=O)N(C)C)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of dimethylamine (0.017 mL, 0.033 mmol) and DIPEA (10.61 μl, 0.061 mmol) in DMF (0.25 mL) was added, and the reaction stirred for a further 1 h. The solvent was removed under a stream of nitrogen. The sample was dissolved in 1:1 MeOH:DMSO (0.9 mL) and purified by MDAP (Method B). The solvent was evaporated in vacuo to give the title compound as an orange oil (5.9 mg, 0.02 mmol, 55%). LCMS (System B): t_{RET}=0.75 min; MH⁺ 351.

### Example 43: 5-(1-benzyl-4-(morpholine-4-carbonyl)-1H-imidazol-2-yl)-1,3-dimethylpyridin-2(1H)-one

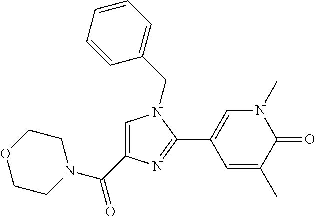 [SMILES: Cc1cc(-c2nc(C(=O)N3CCOCC3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture was stirred under an atmosphere of nitrogen at RT for 1 h. A solution of morpholine (2.91 mg, 0.033 mmol) and DIPEA (10.61 μl, 0.061 mmol) in DMF (0.25 mL) was added, and the reaction stirred for a further 1 h. The solvent was removed under a stream of nitrogen. The sample was dissolved in 1:1 MeOH:DMSO (0.9 mL) and purified by MDAP (Method B). The solvent was evaporated in vacuo to give the title compound as a white solid (6.0 mg, 0.02 mmol, 50%). LCMS (System B): t_{RET}=0.75 min; MH⁺ 393.

### Example 44: 5-(1-benzyl-4-(pyrrolidine-1-carbonyl)-1H-imidazol-2-yl)-1,3-dimethylpyridin-2(1H)-one

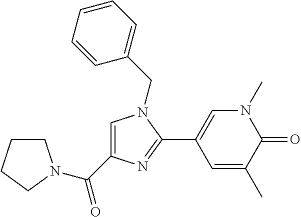 [SMILES: Cc1cc(-c2nc(C(=O)N3CCCC3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture was stirred under an atmosphere of nitrogen at RT for 1 h. A solution of pyrrolidine (4.32 mg, 0.061 mmol) and DIPEA (10.61 μl, 0.061 mmol) in DMF (0.25 mL) was added, and the reaction stirred for a further 1 h. The sample was diluted to 0.9 mL with methanol and purified by MDAP (Method B). The solvent was evaporated in vacuo to give the title compound as a colourless oil (5.6 mg, 0.02 mmol, 49%). LCMS (System B): t_{RET}=0.82 min; MH⁺ 377.

### Example 45: 5-(4-(azetidine-1-carbonyl)-1-benzyl-1H-imidazol-2-yl)-1,3-dimethylpyridin-2(1H)-one

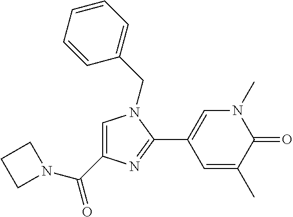 [SMILES: Cc1cc(-c2nc(C(=O)N3CCC3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture was stirred under an atmosphere of nitrogen at RT for 1 h. A solution of azetidine (3.47 mg, 0.061 mmol) and DIPEA (10.61 μl, 0.061 mmol) in DMF (0.25 mL) was added, and the reaction stirred for a further 1 h. The sample was diluted to 0.9 mL with methanol and purified by MDAP (Method B). The solvent was evaporated in vacuo to give the title compound as a colourless oil (6.2 mg, 0.02 mmol, 56%). LCMS (System B): t_{RET}=0.76 min; MH⁺ 363.

### Example 46: 1-benzyl-2-(1,5-dimethyl-6-oxo-1,6-dihydropyridin-3-yl)-N-methyl-1H-imidazole-4-carboxamide

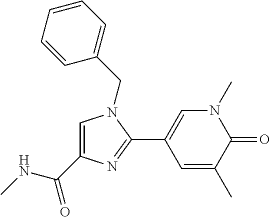 [SMILES: CNC(=O)c1cn(Cc2ccccc2)c(-c2cc(C)c(=O)n(C)c2)n1]

Lithium 1-benzyl-2-(1,5-dimethyl-6-oxo-1,6-dihydropyridin-3-yl)-1H-imidazole-4-carboxylate (for an example preparation, see Intermediate 13, 50 mg, 0.152 mmol) was added to a vial containing a stirrer bar, and dissolved in DMF (1.25 mL). HATU (63.5 mg, 0.167 mmol) was added, and the reaction mixture was stirred under an atmosphere of nitrogen at RT for 1 h. A solution of methylamine (0.152 mL, 0.304 mmol) and DIPEA (10.61 μl, 0.061 mmol) in DMF (1.25 mL) was added, and the reaction stirred for a further 1 h. The solvent was removed under reduced pressure. The sample was dissolved in 1:1 MeOH:DMSO (0.9 mL) and purified by MDAP (Method B). The solvent was evaporated in vacuo to give the title compound as a white solid (30.7 mg, 0.09 mmol, 60%). LCMS (System B): t_{RET}=0.72 min; MH⁺ 337.

### Example 47: 5-(1-benzyl-5-chloro-1H-imidazol-2-yl)-1,3-dimethylpyridin-2(1H)-one

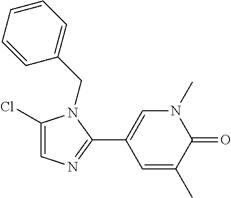 [SMILES: Cc1cc(-c2ncc(Cl)n2Cc2ccccc2)cn(C)c1=O]

5-(4-Chloro-1H-imidazol-2-yl)-1,3-dimethylpyridin-2(1H)-one (for an example preparation, see Intermediate 12, 74 mg, 0.331 mmol) was dissolved in DMF (1 mL) in a round bottomed flask and placed in an ice bath. With the solution at 0° C., sodium hydride (60% dispersion in mineral oil) (17.20 mg, 0.430 mmol) was added and the solution stirred for 30 min. Benzyl bromide (0.047 mL, 0.397 mmol) in DMF (1 mL) was added dropwise to the reaction mixture. The vessel was allowed to warm to RT and the reaction mixture stirred overnight. The reaction mixture was quenched with MeOH (0.5 mL) and the solvent removed under a stream of nitrogen. The samples were dissolved in 3 mL 1:1 MeOH:DMSO and purified by MDAP (Method B). The solvent was evaporated in vacuo and the desired fractions redissolved in 1 mL 1:1 MeOH:DMSO and purified further by MDAP (Method A). The solvent was evaporated in vacuo and the desired fractions redissolved in 1 mL 1:1 MeOH:DMSO and purified finally by MDAP (Method C) to yield the title compound as a pale oil, (9 mg, 0.03 mmol, 13%). LCMS (System C): t_{RET}=0.62 min; MH⁺ 314, 316.

### Example 48: 1-benzyl-2-(1,5-dimethyl-6-oxo-1,6-dihydropyridin-3-yl)-N-ethyl-1H-imidazole-4-carboxamide

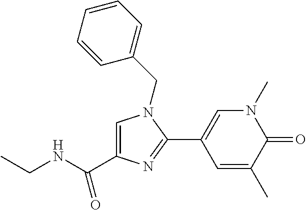 [SMILES: CCNC(=O)c1cn(Cc2ccccc2)c(-c2cc(C)c(=O)n(C)c2)n1]

Lithium 1-benzyl-2-(1,5-dimethyl-6-oxo-1,6-dihydropyridin-3-yl)-1H-imidazole-4-carboxylate (for an example preparation, see Intermediate 13, 50 mg, 0.152 mmol) was added to a vial containing a stirrer bar, and dissolved in DMF (1.25 mL). HATU (63.5 mg, 0.167 mmol) was added, and the reaction mixture stirred under an atmosphere of nitrogen at RT for 1 h. A solution of ethylamine (0.152 mL, 0.304 mmol) and DIPEA (10.61 μl, 0.061 mmol) in DMF (1.25 mL) was added, and the reaction stirred for a further 1 h. The solvent was removed under reduced pressure. The sample was dissolved in 1:1 MeOH:DMSO (0.9 mL) and purified by MDAP (Method B). The solvent was removed under a stream of nitrogen to give the title compound as a white solid (35.1 mg, 0.10 mmol, 66%). LCMS (System B): t_{RET}=0.78 min; MH⁺ 351.

### Example 49: rac-1-benzyl-2-(1,5-dimethyl-6-oxo-1,6-dihydropyridin-3-yl)-N-(tetrahydrofuran-3-yl)-1H-imidazole-4-carboxamide

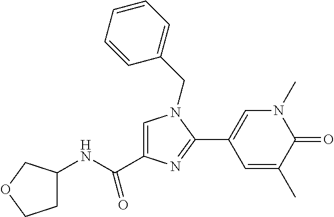 [SMILES: Cc1cc(-c2nc(C(=O)NC3CCOC3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of rac-tetrahydrofuran-3-amine (5.29 mg, 0.061 mmol) and DIPEA (10.61 μl, 0.061 mmol) in DMF (0.25 mL) was added, and the reaction stirred for a further 1 h. The sample was diluted to 0.9 mL with methanol and purified by MDAP (Method B). The solvent was evaporated in vacuo to give the title compound as a colourless oil (8.0 mg, 0.02 mmol, 67%). LCMS (System B): t_{RET}=0.75 min; MH⁺ 393.

### Example 50: 5-(1-benzyl-4-(piperidine-1-carbonyl)-1H-imidazol-2-yl)-1,3-dimethylpyridin-2(1H)-one

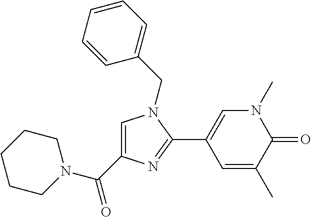 [SMILES: Cc1cc(-c2nc(C(=O)N3CCCCC3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of piperidine (5.17 mg, 0.061 mmol) and DIPEA (10.61 μl, 0.061 mmol) in DMF (0.25 mL) was added, and the reaction stirred for a further 1 h. The sample was diluted to 0.9 mL with methanol and purified by MDAP (Method B). The solvent was evaporated in vacuo to give the title compound as an off-white oil (10.0 mg, 0.03 mmol, 84%). LCMS (System B): t_{RET}=0.89 min; MH⁺ 391.

### Example 51: 1-benzyl-2-(1,5-dimethyl-6-oxo-1,6-dihydropyridin-3-yl)-N-(tetrahydro-2H-pyran-4-yl)-1H-imidazole-4-carboxamide

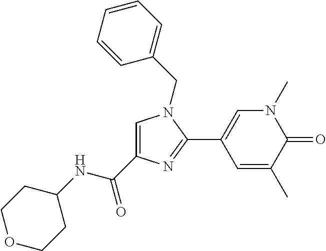 [SMILES: Cc1cc(-c2nc(C(=O)NC3CCOCC3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of tetrahydro-2H-pyran-4-amine, hydrochloride salt (8.36 mg, 0.061 mmol) and DIPEA (10.61 μl, 0.061 mmol) in DMF (0.25 mL) was added, and the reaction stirred for a further 1 h. The sample was diluted to 0.9 mL with methanol and purified by MDAP (Method B). The solvent was evaporated in vacuo to give the title compound as an colourless oil (4.5 mg, 0.01 mmol, 37%). LCMS (System B): t_{RET}=0.78 min; MH⁺ 407.

### Example 52: 1-benzyl-2-(1,5-dimethyl-6-oxo-1,6-dihydropyridin-3-yl)-N-phenethyl-1H-imidazole-4-carboxamide

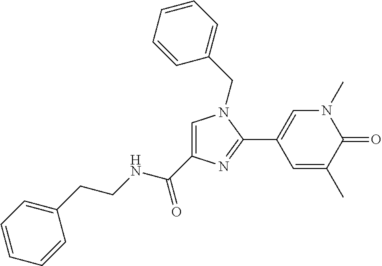 [SMILES: Cc1cc(-c2nc(C(=O)NCCc3ccccc3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of 2-phenethylamine (7.36 mg, 0.061 mmol) and DIPEA (10.61 μl, 0.061 mmol) in DMF (0.25 mL) was added, and the reaction stirred for a further 1 h. The sample was diluted to 0.9 mL with methanol and purified by MDAP (Method B). The solvent was evaporated in vacuo to give the title compound as an pale yellow oil (5.2 mg, 0.01 mmol, 40%). LCMS (System B): t_{RET}=1.00 min; MH⁺ 427.

### Example 53: N,1-dibenzyl-2-(1,5-dimethyl-6-oxo-1,6-dihydropyridin-3-yl)-1H-imidazole-4-carboxamide

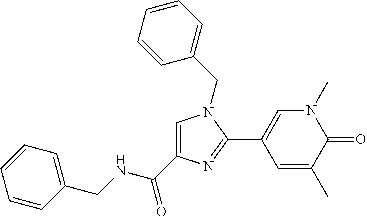 [SMILES: Cc1cc(-c2nc(C(=O)NCc3ccccc3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of benzylamine (6.51 mg, 0.061 mmol) and DIPEA (10.61 μl, 0.061 mmol) in DMF (0.25 mL) was added, and the reaction stirred for a further 1 h. The sample was diluted to 0.9 mL with methanol and purified by MDAP (Method B). The solvent was evaporated in vacuo to give the title compound as a white solid (4.3 mg, 0.01 mmol, 34%). LCMS (System B): t_{RET}=0.96 min; MH⁺ 413.

### Examples 54 and 55: 1-benzyl-2-(1,5-dimethyl-6-oxo-1,6-dihydropyridin-3-yl)-1H-imidazole-4-carbonitrile & 1-benzyl-2-(1,5-dimethyl-6-oxo-1,6-dihydropyridin-3-yl)-1H-imidazole-5-carbonitrile

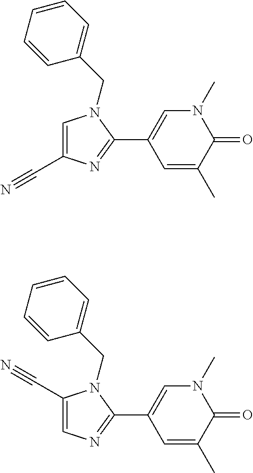 [SMILES: Cc1cc(-c2nc(C#N)cn2Cc2ccccc2)cn(C)c1=O ; Cc1cc(-c2ncc(C#N)n2Cc2ccccc2)cn(C)c1=O]

2-(1,5-dimethyl-6-oxo-1,6-dihydropyridin-3-yl)-1H-imidazole-4-carbonitrile (for an example preparation, see Intermediate 18, 428 mg, 0.380 mmol) was dissolved in DMF (2 mL) in a round bottomed flask in an ice bath. With the solution at 0° C., sodium hydride (60% dispersion in mineral oil) (19.74 mg, 0.493 mmol) was added and the solution stirred for 30 min. A solution of benzyl bromide (0.054 mL, 0.456 mmol) in DMF (1 mL) was added dropwise. Once all the reactant was added, the reaction mixture was allowed to warm to RT and stirred overnight. The reaction mixture was cooled to 0° C. and further sodium hydride (60% dispersion in mineral oil) (19.74 mg, 0.493 mmol) was added. After 30 min, a solution of benzyl bromide (78 mg, 0.456 mmol) in DMF (1 mL) was added and the reaction mixture allowed to warm to RT before stirring overnight under an atmosphere of nitrogen. The reaction mixture was again cooled to 0° C. and purged with nitrogen prior to the addition of further sodium hydride (60% dispersion in mineral oil) (45.5 mg, 1.139 mmol). The vessel was sealed, and, after 30 min, a solution of benzyl bromide (195 mg, 1.139 mmol) in DMF (0.5 mL) was added and the reaction mixture allowed to warm to RT with overnight stirring. The mixture was quenched with methanol (0.5 mL) and the solvent removed under a stream of nitrogen. The residue was dissolved in 6 mL 1:1 MeOH:DMSO and purified by MDAP (Method B). The solvent was evaporated in vacuo to give the crude product. The sample (160 mg) was purified further by HPLC. The sample was dissolved in 12 mL DMSO and 3000 uL injections were made onto a CSH C18 150×30 mm, 5 um column and eluted using a gradient of 30-99% MeOH in 0.1% formic acid/water over 41 min at a flow rate of 40 mL/min. Fractions containing each of the separated isomeric products (determined by diode array & mass spec) were combined and dried under a stream of nitrogen. The title compound (Example 54) was the major regioisomer formed in the reaction, and was afforded as a colourless oil (90 mg, 0.30 mmol, 78%). LCMS (System C,): t_{RET}=0.81 min; MH⁺ 305.

The alternative title compound (Example 55) was the minor regioisomer formed in the reaction, and was afforded as the formate salt, a white solid (10 mg, 0.03 mmol, 8%). LCMS (System C): t_{RET}=0.79 min; MH⁺ 305.

### Example 56: 5-(1-benzyl-4-bromo-5-chloro-1H-imidazol-2-yl)-1,3-dimethylpyridin-2(1H)-one

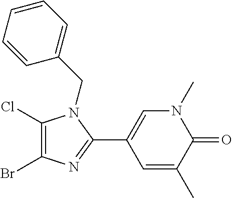 [SMILES: Cc1cc(-c2nc(Br)c(Cl)n2Cc2ccccc2)cn(C)c1=O]

1,3-Dimethyl-5-(4,4,5,5-tetramethyl-1,3,2-dioxaborolan-2-yl)pyridin-2(1H)-one (7.17 mg, 0.029 mmol), 1-benzyl-2,4-dibromo-5-chloro-1H-imidazole (for an example preparation, see Intermediate 20, 8.4 mg, 0.024 mmol), and potassium carbonate (8.28 mg, 0.060 mmol) were added to a microwave vial. 1,4-Dioxane (0.4 mL) and water (0.13 mL) were added to the vial, which was purged with nitrogen for 5 min prior to the addition of tetrakis(triphenylphosphine)palladium(0) (0.831 mg, 0.719 μmol). After a further 5 min purge with nitrogen, the vial was heated in a microwave at 110° C. for 1 h. The solvent was removed under a stream of nitrogen and the residue redissolved in EtOAc and filtered. The solvent was removed from the filtrate under reduced pressure. The sample was dissolved in 1:1 MeOH:DMSO (0.9 mL) and purified by MDAP (Method B). The solvent was removed under a stream of nitrogen to afford the title compound as an orange oil (3.5 mg, 8.91 μmol, 37%). LCMS (System B): t_{RET}=1.09 min; MH⁺ 392, 394, 396.

### Example 57: N-(1-benzyl-2-(1,5-dimethyl-6-oxo-1,6-dihydropyridin-3-yl)-1H-imidazol-4-yl)acetamide

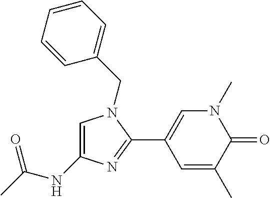 [SMILES: CC(=O)Nc1cn(Cc2ccccc2)c(-c2cc(C)c(=O)n(C)c2)n1]

Iron (517 mg, 9.25 mmol) and acetic acid (11 mL) were added to a vial containing 5-(1-benzyl-4-nitro-1H-imidazol-2-yl)-1,3-dimethylpyridin-2(1H)-one (for an example preparation, see Example 40, 300 mg, 0.925 mmol), and the reaction mixture was stirred at 75° C. for 5 h. The solvent was removed in vacuo, before the residue was dissolved in EtOAc (10 mL) and filtered through Celite®, washing with further EtOAc (3×10 mL). The solvent was removed in vacuo and the residue purified by MDAP (Method B) to afford the title compound as a colourless solid (129 mg, 0.364 mmol, 39%). LCMS (System B): t_{RET}=0.74 min; MH⁺ 337.

### Example 58: 5-(4-chloro-1-(pyridin-2-ylmethyl)-1H-imidazol-2-yl)-1,3-dimethylpyridin-2(1H)-one

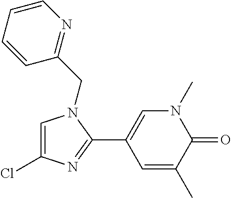 [SMILES: Cc1cc(-c2nc(Cl)cn2Cc2ccccn2)cn(C)c1=O]

To a stirred solution of 5-(4-chloro-1H-imidazol-2-yl)-1,3-dimethylpyridin-2(1H)-one (for an example preparation, see Intermediate 12, 40 mg, 0.179 mmol) in DMF (2 mL) at 0° C. was added sodium hydride (17.88 mg, 0.447 mmol). The reaction was stirred for 30 min, then 2-(bromomethyl)pyridine, hydrobromide salt (67.9 mg, 0.268 mmol) was added. The reaction mixture was allowed to warm to RT and stirred for a further 18 h. The solvent was removed in vacuo and the crude residue redissolved in 1:1 DMSO:MeOH (1.8 mL), filtered through a syringe filter, and purified by MDAP (Method A) to afford the title compound as a cream solid (35.4 mg, 0.107 mmol, 60%). LCMS (System A): t_{RET}=0.68 min; MH⁺ 315, 317.

### Example 59: 1-benzyl-2-(1,5-dimethyl-6-oxo-1,6-dihydropyridin-3-yl)-1H-imidazole-4-carboxamide

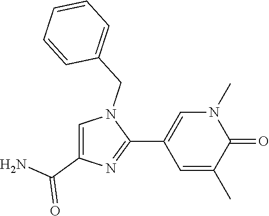 [SMILES: Cc1cc(-c2nc(C(N)=O)cn2Cc2ccccc2)cn(C)c1=O]

2-(1,5-dimethyl-6-oxo-1,6-dihydropyridin-3-yl)-1H-imidazole-4-carboxamide (for an example preparation, see Intermediate 21, 100 mg, 0.431 mmol) was dissolved in DMF (1 mL) in a vial in an ice bath, and purged with nitrogen for 10 min. With the solution at 0° C., sodium hydride (60% dispersion in mineral oil) (22.39 mg, 0.560 mmol) was added and the solution stirred for 30 min. A solution of benzyl bromide (0.061 mL, 0.517 mmol) in DMF (1 mL) was added dropwise to the reaction mixture. Once all reactant was added, the reaction mixture was allowed to warm to RT and stirred overnight under an atmosphere of nitrogen. The mixture was quenched with MeOH (0.5 mL) and the solvent removed under reduced pressure. The sample was dissolved in 1:1 MeOH:DMSO (1.8 mL) and purified by MDAP (Method B). The solvent was removed under a stream of nitrogen to afford the title compound as as a white solid (34 mg, 0.11 mmol, 24%). LCMS (System B): t_{RET}=0.69 min; MH⁺ 323.

### Examples 60 and 61: rac-5-(4-chloro-1-(1-(pyridin-2-yl)ethyl)-1H-imidazol-2-yl)-1,3-dimethylpyridin-2(1H)-one & rac-5-(5-chloro-1-(1-(pyridin-2-yl)ethyl)-1H-imidazol-2-yl)-1,3-dimethylpyridin-2(1H)-one

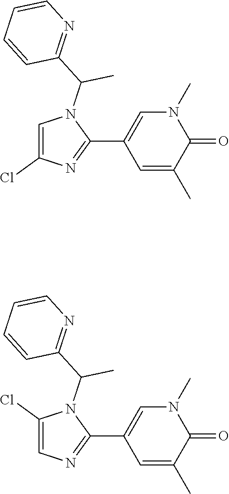 [SMILES: Cc1cc(-c2nc(Cl)cn2C(C)c2ccccn2)cn(C)c1=O ; Cc1cc(-c2ncc(Cl)n2C(C)c2ccccn2)cn(C)c1=O]

To a stirred solution of 5-(4-chloro-1H-imidazol-2-yl)-1,3-dimethylpyridin-2(1H)-one (for an example preparation, see Intermediate 12, 40 mg, 0.161 mmol) in DMF (2 mL) at 0° C. was added sodium hydride (60% dispersion in mineral oil) (25.8 mg, 0.644 mmol). The reaction was stirred for 30 min, then rac-2-(1-bromoethyl)pyridine, hydrobromide salt (64.5 mg, 0.241 mmol) was added. The reaction was allowed to warm to RT and stirred overnight. Further rac-2-(1-bromoethyl)pyridine, hydrobromide salt (80 mg, 0.299 mmol) and sodium hydride (60% dispersion in mineral oil) (25.8 mg, 0.644 mmol) were added and the reaction was left to stir at RT for a further day. The solvent was removed from the reaction mixture in vacuo. The sample was dissolved in 1:1 MeOH:DMSO (1 mL) and purified by MDAP (Method A). Fractions containing each of the separated isomeric products were combined and the solvent was removed in vacuo. The major isomer 60 was dissolved in MeOH and purified by solid phase extraction on a sulphonic acid (SCX) cartridge with sequential solvent elution of methanol and 2M ammonia in methanol. Product containing fractions were combined and the solvent removed in vacuo, then dried further under a stream of nitrogen to afford the title compound (Example 60) as the major regioisomer from the reaction (18 mg, 0.06 mmol, 34%). LCMS (System A): t_{RET}=0.77 min; MH⁺ 329. The minor isomer (Example 61) was dissolved in 1:1 MeOH:DMSO (1 mL) and purified further by MDAP (Method B). The solvent was removed in vacuo to afford the title compound 61 (2.6 mg, 7.91 μmol, 5%). LCMS (System A): t_{RET}=0.59 min; MH⁺ 329, 331.

### Example 62: 5-(1-benzyl-4-fluoro-1H-imidazol-2-yl)-1,3-dimethylpyridin-2(1H)-one

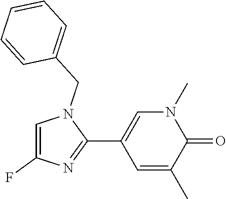 [SMILES: Cc1cc(-c2nc(F)cn2Cc2ccccc2)cn(C)c1=O]

A solution of TMPMgCl.LiCl (1 M in THF/toluene, 0.511 mL, 0.511 mmol) was added dropwise to a solution of 1-benzyl-4-fluoro-1H-imidazole (for an example preparation, see Intermediate 22, 45 mg, 0.255 mmol) in THF (1 mL) at 0° C. in a dried vial under nitrogen. The reaction mixture was stirred at 0° C. for 30 min, then a solution of zinc chloride (1.9M in 2-methyltetrahydrofuran) (0.403 mL, 0.766 mmol) was added, and the reaction stirred at 0° C. for a further 15 min. The reaction was allowed to warm to RT and added via syringe to a dried and nitrogen-purged vial containing 5-bromo-1,3-dimethylpyridin-2(1H)-one (103 mg, 0.511 mmol) and bis(triphenylphosphine)palladium dichloride (14.34 mg, 0.020 mmol). The reaction mixture was then heated at 110° C. in a Biotage I60 microwave reactor for 60 min. The reaction was quenched by the addition of MeOH (1 mL) and the solvent removed in vacuo. The crude residue was dissolved in MeOH (1.8 mL), filtered through a pipette filter, and purified by MDAP (Method B) to afford the title compound as a colourless film (26 mg, 0.08 mmol, 33%). LCMS (System A): t_{RET}=0.88 min; MH⁺ 298.

### Example 63: 5-(1-benzyl-4-bromo-1H-imidazol-2-yl)-1,3-dimethylpyridin-2(1H)-one

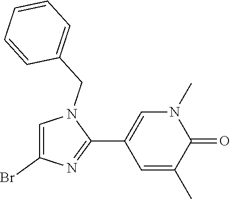 [SMILES: Cc1cc(-c2nc(Br)cn2Cc2ccccc2)cn(C)c1=O]

1-benzyl-2,4-dibromo-1H-imidazole (for an example preparation, see Intermediate 23, 1.16 g, 3.67 mmol), 1,3-dimethyl-5-(4,4,5,5-tetramethyl-1,3,2-dioxaborolan-2-yl)pyridin-2(1H)-one (commercially available from, for example, Milestone PharmaTech, 1.097 g, 4.41 mmol), potassium carbonate (1.268 g, 9.18 mmol), 1,4-dioxane (11.75 mL) and water (2.94 mL) were added to a microwave vial and purged with nitrogen for 5 min. Tetrakis(triphenylphosphine)palladium(0) (0.127 g, 0.110 mmol) was added, the vial sealed, and the reaction mixture purged with nitrogen for a further 5 min. The reaction was heated at 110° C. in a microwave reactor for 1 h. The solvent was removed in vacuo, the crude residue redissolved in EtOAc (20 mL) and filtered through Celite®, washing with further EtOAc (3×20 mL). The solvent was removed in vacuo, the residue dissolved in DCM (10 mL) and purified by flash column chromatography on a Combiflash Rf+ using an elution gradient of 0-100% EtOAc in cyclohexane over 30 CV (120 g silica cartridge). The appropriate fractions were combined and the solvent removed in vacuo to afford the title compound as a light yellow solid (638 mg, 1.60 mmol, 44%). 100 mg of this product was repurified by MDAP (Method B) to afford of the title compound 41 mg as a colourless solid. LCMS (System B): t_{RET}=0.95 min; MH⁺ 358, 360.

### Example 64: 5-(1-benzyl-4-methyl-1H-imidazol-2-yl)-1,3-dimethylpyridin-2(1H)-one

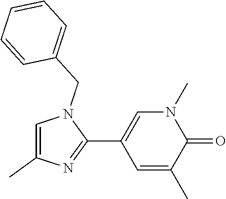 [SMILES: Cc1cn(Cc2ccccc2)c(-c2cc(C)c(=O)n(C)c2)n1]

5-(1-Benzyl-4-bromo-1H-imidazol-2-yl)-1,3-dimethylpyridin-2(1H)-one (for an example preparation, see Example 63, 50 mg, 0.140 mmol), tetrakis(triphenylphosphine)palladium(0) (16.13 mg, 0.014 mmol), and potassium carbonate (57.9 mg, 0.419 mmol) were dissolved in 1,4-dioxane (1 mL), to which trimethylboroxine (0.020 mL, 0.140 mmol) was added dropwise. The flask was evacuated and backfilled with nitrogen 3 times, and the reaction mixture heated at 110° C. in a microwave reactor for 1 h. The solvent was removed in vacuo and the residue dissolved in 1:1 DMSO:MeOH (1.8 mL) and purified by MDAP (Method B) to afford the title compound (19 mg, 0.06 mmol, 44%). LCMS (System B): t_{RET}=0.86 min; MH⁺ 294.

### Example 65: 5-(4-chloro-1-((5-methoxypyridin-2-yl)methyl)-1H-imidazol-2-yl)-1,3-dimethylpyridin-2(1H)-one

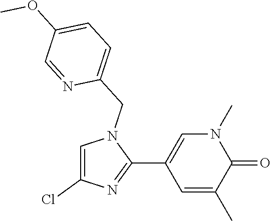 [SMILES: COc1ccc(Cn2cc(Cl)nc2-c2cc(C)c(=O)n(C)c2)nc1]

To a stirred solution of (5-methoxypyridin-2-yl)methanol (49.8 mg, 0.358 mmol) in DMF (0.5 mL) at 0° C. was added sodium hydride (60% dispersion in mineral oil) (10.73 mg, 0.447 mmol) and the reaction mixture stirred for 15 min prior to the addition of 4-methylbenzene-1-sulfonyl chloride (85 mg, 0.447 mmol). The reaction was stirred for a further 30 min at this temperature. In a separate flask, 5-(4-chloro-1H-imidazol-2-yl)-1,3-dimethylpyridin-2(1H)-one (for an example preparation, see Intermediate 12, 40 mg, 0.179 mmol) was dissolved in DMF (0.5 mL) at 0° C., to which sodium hydride (60% dispersion in mineral oil) (8.58 mg, 0.358 mmol) was added and the reaction stirred for a further 30 min. The solution containing the tosylated pyridine was added dropwise to the flask containing the imidazole, and the reaction stirred for a further 3 h. The reaction was quenched with MeOH and the solvent removed in vacuo. The crude product was purified by MDAP (Method C) to afford the title compound as the trifluoroacetic acid salt, a light brown oil (27 mg, 0.06 mmol, 33%). LCMS (System A): t_{RET}=0.75 min; MH⁺ 345.

### Example 66: 5-(4-chloro-1-((6-methoxypyridin-3-yl)methyl)-1H-imidazol-2-yl)-1,3-dimethylpyridin-2(1H)-one

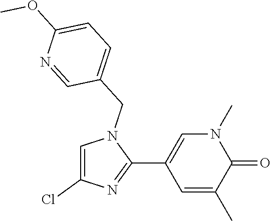 [SMILES: COc1ccc(Cn2cc(Cl)nc2-c2cc(C)c(=O)n(C)c2)cn1]

To a stirred solution of (6-methoxypyridin-3-yl)methanol (49.8 mg, 0.358 mmol) in DMF (0.5 mL) at 0° C. was added sodium hydride (60% dispersion in mineral oil) (10.73 mg, 0.447 mmol) and the reaction mixture stirred for 15 min prior to the addition of 4-methylbenzene-1-sulfonyl chloride (85 mg, 0.447 mmol). The reaction was stirred for a further 30 min at this temperature. In a separate flask, 5-(4-chloro-1H-imidazol-2-yl)-1,3-dimethylpyridin-2(1H)-one (for an example preparation, see Intermediate 12, 40 mg, 0.179 mmol) was dissolved in DMF (0.5 mL) at 0° C., to which sodium hydride (60% dispersion in mineral oil) (8.58 mg, 0.358 mmol) was added and the reaction stirred for a further 30 min. The solution containing the tosylated pyridine was added dropwise to the flask containing the imidazole, and the reaction stirred for a further 16 h. The solvent was removed in vacuo and the crude product was purified by MDAP (Method A) to afford the title compound as a colourless film (16 mg, 0.04 mmol, 23%). LCMS (System A): t_{RET}=0.79 min; MH⁺ 345.

### Example 67: 5-(1-benzyl-4-phenyl-1H-imidazol-2-yl)-1,3-dimethylpyridin-2(1H)-one

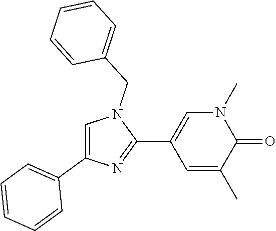 [SMILES: Cc1cc(-c2nc(-c3ccccc3)cn2Cc2ccccc2)cn(C)c1=O]

1,5-dimethyl-6-oxo-1,6-dihydropyridine-3-carbaldehyde (50 mg, 0.331 mmol), 2-bromo-1-phenylethanone (65.8 mg, 0.331 mmol), phenylmethanamine (35.4 mg, 0.331 mmol) and ammonium acetate (38.2 mg, 0.496 mmol) were added to a round bottomed flask, and the reaction mixture was stirred at 130° C. for 2 h. The reaction mixture was dissolved in 1:1 MeOH:DMSO (0.9 mL) and purified by MDAP (Method A). The solvent was evaporated in vacuo and the residue was redissolved up in 30 mL EtOAc and washed with brine solution (2×20 mL). The organic layer was passed through a hydrophobic frit and the solvent removed under reduced pressure. The residue was dissolved in 1:1 MeOH:DMSO (0.9 mL) and purified by MDAP (Method B). The solvent was dried under a stream of nitrogen to afford the title compound as a yellow oil (7.2 mg, 0.02 mmol, 6%). LCMS (System B): t_{RET}=1.08 min; MH⁺ 356.

### Example 68: 1-benzyl-N-cyclopropyl-2-(1,5-dimethyl-6-oxo-1,6-dihydropyridin-3-yl)-1H-imidazole-4-carboxamide

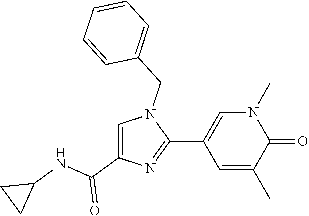 [SMILES: Cc1cc(-c2nc(C(=O)NC3CC3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of cyclopropylamine (3.47 mg, 0.061 mmol) and DIPEA (10.61 μl, 0.061 mmol) in DMF (0.25 mL) was added, and the reaction stirred for a further 1 h. The solvent was removed under reduced pressure. The sample was dissolved in 1:1 MeOH:DMSO (0.9 mL) and purified by MDAP (Method B). The solvent was removed under a stream of nitrogen to give the title compound as a pale yellow oil (6.8 mg, 0.02 mmol, 62%). LCMS (System B): t_{RET}=0.81 min; MH⁺ 363.

### Example 69: 1-benzyl-2-(1,5-dimethyl-6-oxo-1,6-dihydropyridin-3-yl)-N-isopropyl-1H-imidazole-4-carboxamide

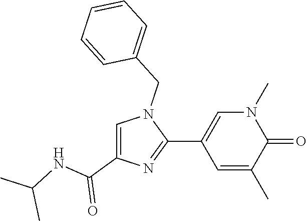 [SMILES: Cc1cc(-c2nc(C(=O)NC(C)C)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of propan-2-amine (3.59 mg, 0.061 mmol) and DIPEA (10.61 μl, 0.061 mmol) in DMF (0.25 mL) was added, and the reaction stirred for a further 1 h. The solvent was removed under reduced pressure. The sample was dissolved in 1:1 MeOH:DMSO (0.9 mL) and purified by MDAP (Method B). The solvent was removed under a stream of nitrogen to give the title compound as an off-white solid (6.6 mg, 0.02 mmol, 60%). LCMS (System B): t_{RET}=0.86 min; MH⁺ 365.

### Example 70: 1-benzyl-2-(1,5-dimethyl-6-oxo-1,6-dihydropyridin-3-yl)-N-((tetrahydrofuran-2-yl)methyl)-1H-imidazole-4-carboxamide

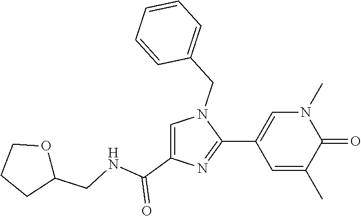 [SMILES: Cc1cc(-c2nc(C(=O)NCC3CCCO3)cn2Cc2ccccc2)cn(C)c1=O]

Lithium 1-benzyl-2-(1,5-dimethyl-6-oxo-1,6-dihydropyridin-3-yl)-1H-imidazole-4-carboxylate (for an example preparation, see Intermediate 13, 10 mg, 0.030 mmol) was added to a vial containing a stirrer bar, and dissolved in DMF (0.25 mL). HATU (12.70 mg, 0.033 mmol) was added, and the reaction mixture stirred under an atmosphere of nitrogen at RT for 1 h. A solution of (tetrahydrofuran-2-yl)methylamine (6.14 mg, 0.061 mmol) and DIPEA (10.61 μl, 0.061 mmol) in DMF (0.25 mL) was added, and the reaction stirred for a further 1 h. The solvent was removed under reduced pressure. The sample was dissolved in 1:1 MeOH:DMSO (0.9 mL) and purified by MDAP (Method B). The solvent was removed under a stream of nitrogen to give the title compound as an off-white solid (7.5 mg, 0.02 mmol, 61%). LCMS (System B): t_{RET}=0.78 min; MH⁺ 407.

### Example 71: 5-(4-chloro-1-((5-methylpyridin-2-yl)methyl)-1H-imidazol-2-yl)-1,3-dimethylpyridin-2(1H)-one

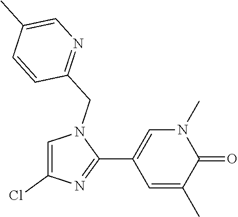 [SMILES: Cc1ccc(Cn2cc(Cl)nc2-c2cc(C)c(=O)n(C)c2)nc1]

To a stirred solution of (5-methylpyridin-2-yl)methanol (44.1 mg, 0.358 mmol) in DMF (0.5 mL) at 0° C. was added sodium hydride (10.73 mg, 0.447 mmol) and the reaction mixture stirred for 15 min prior to addition of 4-methylbenzene-1-sulfonyl chloride (85 mg, 0.447 mmol). The reaction was stirred for a further 30 min at this temperature. In a separate flask, 5-(4-chloro-1H-imidazol-2-yl)-1,3-dimethylpyridin-2(1H)-one (for an example preparation, see Intermediate 12, 40 mg, 0.179 mmol) was dissolved in DMF (0.5 mL) at 0° C., to which sodium hydride (8.58 mg, 0.358 mmol) was added and the reaction stirred for a further 30 min. The solution containing the tosylated pyridine was added dropwise to the flask containing the imidazole, and the reaction stirred for a further 16 h. The reaction was quenched with MeOH and the solvent removed in vacuo. The crude residue was dissolved in 1:1 DMSO:MeOH (0.9 mL) and purified by MDAP (Method C) to afford the title compound as a colourless solid (13.0 mg, 0.04 mmol, 22%). LCMS (System A): t_{RET}=0.75 min; MH⁺ 329, 331

### Example 72: 5-(4-chloro-1-((5-fluoropyridin-2-yl)methyl)-1H-imidazol-2-yl)-1,3-dimethylpyridin-2(1H)-one

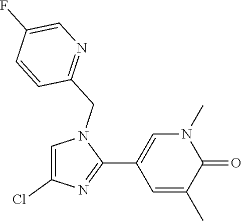 [SMILES: Cc1cc(-c2nc(Cl)cn2Cc2ccc(F)cn2)cn(C)c1=O]

To a stirred solution of (5-fluoropyridin-2-yl)methanol (45.5 mg, 0.358 mmol) in DMF (0.5 mL) at 0° C. was added sodium hydride (10.73 mg, 0.447 mmol) and the reaction mixture stirred for 15 min prior to addition of 4-methylbenzene-1-sulfonyl chloride (85 mg, 0.447 mmol). The reaction was stirred for a further 30 min at this temperature. In a separate flask, 5-(4-chloro-1H-imidazol-2-yl)-1,3-dimethylpyridin-2(1H)-one (for an example preparation, see Intermediate 12, 40 mg, 0.179 mmol) was dissolved in DMF (0.5 mL) at 0° C., to which sodium hydride (8.58 mg, 0.358 mmol) was added and the reaction stirred for a further 30 min. The solution containing the tosylated pyridine was added dropwise to the flask containing the imidazole, and the reaction stirred for a further 16 h. The reaction was quenched with MeOH and the solvent removed in vacuo. The crude residue was dissolved in 1:1 DMSO:MeOH (0.9 mL) and purified by MDAP (Method C) to afford the title compound as a colourless solid (10.5 mg, 0.03 mmol, 17%). LCMS (System A): t_{RET}=0.77 min; MH⁺ 333, 335.

### Example 73: 5-((4-chloro-2-(1,5-dimethyl-6-oxo-1,6-dihydropyridin-3-yl)-1H-imidazol-1-yl)methyl)picolinonitrile

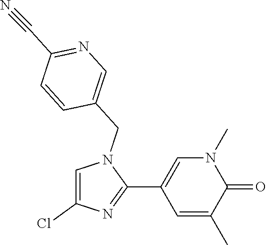 [SMILES: Cc1cc(-c2nc(Cl)cn2Cc2ccc(C#N)nc2)cn(C)c1=O]

To a stirred solution of 5-(4-chloro-1H-imidazol-2-yl)-1,3-dimethylpyridin-2(1H)-one (for an example preparation, see Intermediate 12, 65 mg, 0.262 mmol) in DMF (1 mL) at 0° C. under nitrogen was added sodium hydride (60% dispersion in mineral oil) (20.92 mg, 0.523 mmol). The reaction was stirred for 30 min prior to addition of 5-(bromomethyl)picolinonitrile (103 mg, 0.523 mmol). The reaction mixture was stirred for a further 16 h. The solvent was removed in vacuo and the crude residue redissolved in 1:1 DMSO:MeOH (0.9 mL). Purification by MDAP (Method A) afforded the title compound as the partial formic acid salt, a light brown film (8 mg, 0.02 mmol, 8%). LCMS (System A): t_{RET}=0.72 min; MH⁺ 340, 342.

### Example 74: 1-benzyl-2-(1,5-dimethyl-6-oxo-1,6-dihydropyridin-3-yl)-N-(2-(dimethylamino)ethyl)-1H-imidazole-4-carboxamide

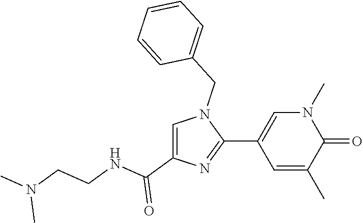 [SMILES: Cc1cc(-c2nc(C(=O)NCCN(C)C)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 198 mg, 0.60 mmol) and HATU (228 mg, 0.60 mmol) was prepared in DMF (3 mL), to which was added DIPEA (0.33 mL, 1.9 mmol). The solution was shaken and 0.5 mL was transferred to a vial containing N¹,N¹-dimethylethane-1,2-diamine (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (23.8 mg, 0.06 mmol, 54%). LCMS (System A): t_{RET}=0.50 min; MH⁺ 394.

### Example 75: 1-benzyl-2-(1,5-dimethyl-6-oxo-1,6-dihydropyridin-3-yl)-N-(2-(N-methylsulfamoyl)ethyl)-1H-imidazole-4-carboxamide

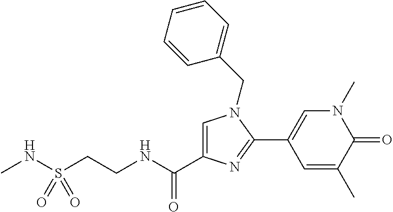 [SMILES: CNS(=O)(=O)CCNC(=O)c1cn(Cc2ccccc2)c(-c2cc(C)c(=O)n(C)c2)n1]

A stock solution of lithium 1-benzyl-2-(1,5-dimethyl-6-oxo-1,6-dihydropyridin-3-yl)-1H-imidazole-4-carboxylate (for an example preparation, see Intermediate 13, 198 mg, 0.60 mmol) and HATU (228 mg, 0.60 mmol) was prepared in DMF (3 mL), to which was added DIPEA (0.33 mL, 1.9 mmol). The solution was shaken and 0.5 mL was transferred to a vial containing 2-amino-N-methylethanesulfonamide (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (24.0 mg, 0.05 mmol, 49%). LCMS (System A): t_{RET}=0.72 min; MH⁺ 444.

### Example 76: 1-benzyl-2-(1,5-dimethyl-6-oxo-1,6-dihydropyridin-3-yl)-N-(2-(pyrrolidin-1-ylsulfonyl)ethyl)-1H-imidazole-4-carboxamide

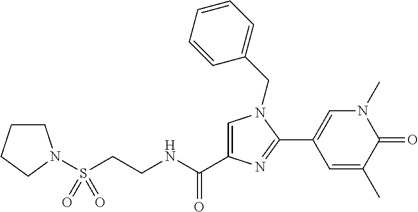 [SMILES: Cc1cc(-c2nc(C(=O)NCCS(=O)(=O)N3CCCC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 198 mg, 0.60 mmol) and HATU (228 mg, 0.60 mmol) was prepared in DMF (3 mL), to which was added DIPEA (0.33 mL, 1.9 mmol). The solution was shaken and 0.5 mL was transferred to a vial containing 2-(pyrrolidin-1-ylsulfonyl)ethanamine (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (24.9 mg, 0.05 mmol, 46%). LCMS (System A): t_{RET}=0.83 min; MH⁺ 484.

### Example 77: 1-benzyl-2-(1,5-dimethyl-6-oxo-1,6-dihydropyridin-3-yl)-N-(2-sulfamoylethyl)-1H-imidazole-4-carboxamide

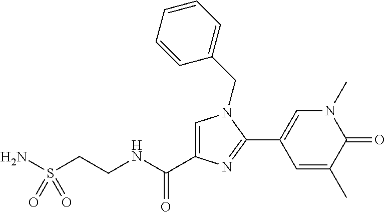 [SMILES: Cc1cc(-c2nc(C(=O)NCCS(N)(=O)=O)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 198 mg, 0.60 mmol) and HATU (228 mg, 0.60 mmol) was prepared in DMF (3 mL), to which was added DIPEA (0.33 mL, 1.9 mmol). The solution was shaken and 0.5 mL was transferred to a vial containing 2-aminoethanesulfonamide, hydrochloride salt (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (29.4 mg, 0.07 mmol, 62%). LCMS (System A): t_{RET}=0.67 min; MH⁺ 430.

### Example 78: 1-benzyl-2-(1,5-dimethyl-6-oxo-1,6-dihydropyridin-3-yl)-N-(2-(piperazin-1-yl)ethyl)-1H-imidazole-4-carboxamide

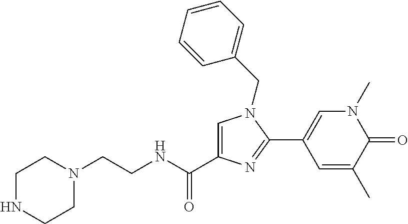 [SMILES: Cc1cc(-c2nc(C(=O)NCCN3CCNCC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 198 mg, 0.60 mmol) and HATU (228 mg, 0.60 mmol) was prepared in DMF (3 mL), to which was added DIPEA (0.33 mL, 1.9 mmol). The solution was shaken and 0.5 mL was transferred to a vial containing tert-butyl 4-(2-aminoethyl)piperazine-1-carboxylate (0.12 mmol). The vial was capped and stood at RT for 2 h. The solvent was removed and the sample redissolved in DMSO (0.5 mL) and purified by MDAP (Method B). The solvent was removed under a stream of nitrogen and the sample dissolved in a solution of DCM (0.5 mL) and 4M HCl in 1,4-dioxane (0.5 mL). The solution was capped and stood at RT for 1 h, after which the solvent was removed to afford the title compound as the hydrochloride salt (34.1 mg, 0.07 mmol, 72%). LCMS (System A): t_{RET}=0.44 min; MH⁺ 435.

### Example 79: 1-benzyl-2-(1,5-dimethyl-6-oxo-1,6-dihydropyridin-3-yl)-N-(piperidin-4-ylmethyl)-1H-imidazole-4-carboxamide

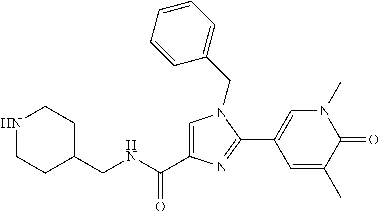 [SMILES: Cc1cc(-c2nc(C(=O)NCC3CCNCC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 198 mg, 0.60 mmol) and HATU (228 mg, 0.60 mmol) was prepared in DMF (3 mL), to which was added DIPEA (0.33 mL, 1.9 mmol). The solution was shaken and 0.5 mL was transferred to a vial containing tert-butyl 4-(aminomethyl)piperidine-1-carboxylate (0.12 mmol). The vial was capped and stood at RT for 2 h. The solvent was removed and the sample redissolved in DMSO (0.5 mL) and purified by MDAP (Method B). The solvent was removed under a stream of nitrogen and the sample dissolved in a solution of DCM (0.5 mL) and 4M HCl in 1,4-dioxane (0.5 mL). The solution was capped and stood at RT for 1 h, after which the solvent was removed to afford the title compound as the hydrochloride salt (26.5 mg, 0.06 mmol, 58%). LCMS (System A): t_{RET}=0.52 min; MH⁺ 420.

### Example 80: rac-1-benzyl-2-(1,5-dimethyl-6-oxo-1,6-dihydropyridin-3-yl)-N-(pyrrolidin-3-ylmethyl)-1H-imidazole-4-carboxamide

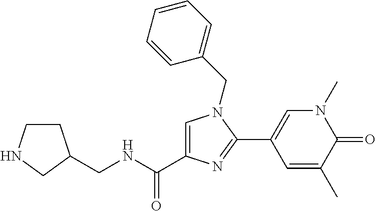 [SMILES: Cc1cc(-c2nc(C(=O)NCC3CCNC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing rac-tert-butyl 3-(aminomethyl)pyrrolidine-1-carboxylate (0.12 mmol). The vial was capped and stood at RT for 2 h. The solvent was removed and the sample redissolved in DMSO (0.5 mL) and purified by MDAP (Method B). The solvent was removed under a stream of nitrogen and the sample dissolved in a solution of DCM (0.5 mL) and HCl in 1,4-dioxane (4M, 0.5 mL). The solution was capped and stood at RT for 1 h, after which the solvent was removed to afford the title compound as the hydrochloride salt (19.4 mg, 0.04 mmol, 40%). LCMS (System A): t_{RET}=0.49 min; MH⁺ 406.

### Example 81: rac-1-benzyl-2-(1,5-dimethyl-6-oxo-1,6-dihydropyridin-3-yl)-N-((tetrahydrofuran-3-yl)methyl)-1H-imidazole-4-carboxamide

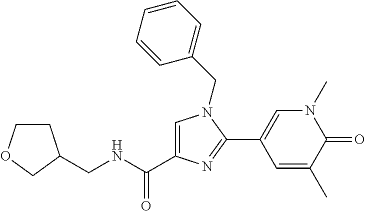 [SMILES: Cc1cc(-c2nc(C(=O)NCC3CCOC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing rac-(tetrahydrofuran-3-yl)methanamine, hydrochloride salt (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (14.3 mg, 0.04 mmol, 32%). LCMS (System A): t_{RET}=0.73 min; MH⁺ 407.

### Example 82: N-((4-(aminomethyl)cyclohexyl)methyl)-1-benzyl-2-(1,5-dimethyl-6-oxo-1,6-dihydropyridin-3-yl)-1H-imidazole-4-carboxamide

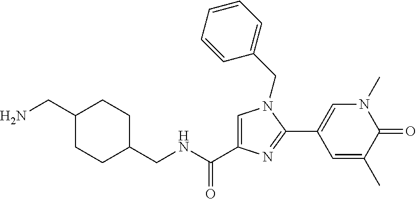 [SMILES: Cc1cc(-c2nc(C(=O)NCC3CCC(CN)CC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing tert-butyl ((4-(aminomethyl)cyclohexyl)methyl)carbamate (0.12 mmol). The vial was capped and stood at RT for 2 h. The solvent was removed and the sample redissolved in DMSO (0.5 mL) and purified by MDAP (Method B). The solvent was removed under a stream of nitrogen and the sample dissolved in a solution of DCM (0.5 mL) and 4M HCl in 1,4-dioxane (0.5 mL). The solution was capped and stood at RT for 1 h, after which the solvent was removed to afford the title compound as the hydrochloride salt (22.9 mg, 0.05 mmol, 43%). LCMS (System A): t_{RET}=0.55 min; MH⁺ 448.

### Example 83: 1-benzyl-2-(1,5-dimethyl-6-oxo-1,6-dihydropyridin-3-yl)-N-(2-morpholinoethyl)-1H-imidazole-4-carboxamide

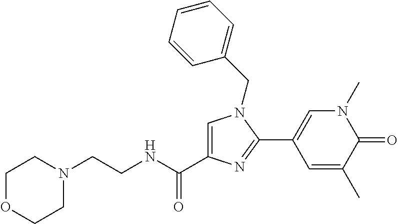 [SMILES: Cc1cc(-c2nc(C(=O)NCCN3CCOCC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing 2-morpholinoethanamine (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (19.8 mg, 0.05 mmol, 41%). LCMS (System A): t_{RET}=0.49 min; MH⁺ 436.

### Example 84: rac-1-benzyl-2-(1,5-dimethyl-6-oxo-1,6-dihydropyridin-3-yl)-N-((1-isopropyl pyrrolidin-3-yl)methyl)-1H-imidazole-4-carboxamide

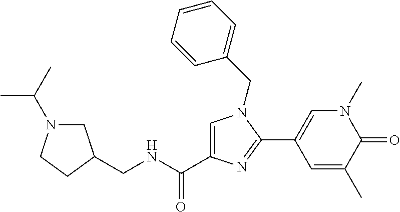 [SMILES: Cc1cc(-c2nc(C(=O)NCC3CCN(C(C)C)C3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.80 mmol). The solution was shaken and 0.5 mL was transferred to a vial containing (1-isopropylpyrrolidin-3-yl)methanamine (0.12 mmol). The vial was capped and stood at RT for 2 h. Propylphosphonic anhydride solution (50% w/w in EtOAc) (0.12 mL, 0.20 mmol) and further DIPEA (55 μL, 0.32 mmol) were added to the vial, which was capped, shaken, and stood at RT for 1 h. The solvent was removed and the sample redissolved in DMSO (0.5 mL) and purified by MDAP (Method B). The solvent was removed under a stream of nitrogen and the resulting sample dissolved in DMSO (1 mL) before repurification by MDAP (Method A). The solvent was removed under a stream of nitrogen to afford the title compound (2.3 mg, 5.1 μmol, 5%). LCMS (System A): t_{RET}=0.52 min; MH⁺ 448.

### Example 85: rac-1-benzyl-2-(1,5-dimethyl-6-oxo-1,6-dihydropyridin-3-yl)-N-(morpholin-2-ylmethyl)-1H-imidazole-4-carboxamide

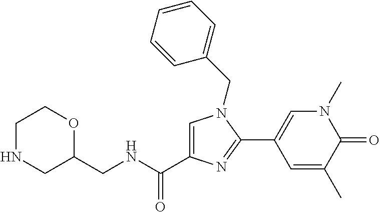 [SMILES: Cc1cc(-c2nc(C(=O)NCC3CNCCO3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing tert-butyl 2-(aminomethyl)morpholine-4-carboxylate (0.12 mmol). The vial was capped and stood at RT for 2 h. The solvent was removed and the sample redissolved in DMSO (0.5 mL) and purified by MDAP (Method B). The solvent was removed under a stream of nitrogen and the sample dissolved in a solution of DCM (0.5 mL) and 4M HCl in 1,4-dioxane (0.5 mL). The solution was capped and stood at RT for 1 h, after which the solvent was removed to afford the title compound (20.8 mg, 0.05 mmol, 44%). LCMS (System A): t_{RET}=0.49 min; MH⁺ 422.

### Example 86: 1-benzyl-2-(1,5-dimethyl-6-oxo-1,6-dihydropyridin-3-yl)-N-((tetrahydro-2H-pyran-4-ylmethyl)-1H-imidazole-4-carboxamide

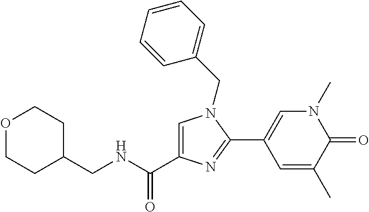 [SMILES: Cc1cc(-c2nc(C(=O)NCC3CCOCC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 132 mg, 0.40 mmol) and HATU (152 mg, 0.40 mmol) was prepared in DMF (2 mL), to which was added DIPEA (0.22 mL, 1.26 mmol). The solution was shaken and 0.5 mL was transferred to a vial containing (tetrahydro-2H-pyran-4-yl)methanamine (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (18.7 mg, 0.04 mmol, 40%). LCMS (System A): t_{RET}=0.76 min; MH⁺ 421.

### Example 87: rac-1-benzyl-2-(1,5-dimethyl-6-oxo-1,6-dihydropyridin-3-yl)-N-((1-methylpyrrolidin-3-yl)methyl)-1H-imidazole-4-carboxamide

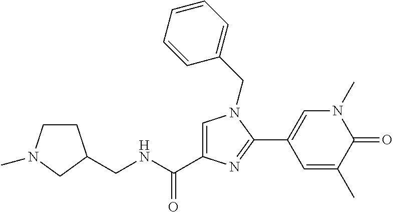 [SMILES: Cc1cc(-c2nc(C(=O)NCC3CCN(C)C3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 132 mg, 0.40 mmol) and HATU (152 mg, 0.40 mmol) was prepared in DMF (2 mL), to which was added DIPEA (0.22 mL, 1.26 mmol). The solution was shaken and 0.5 mL was transferred to a vial containing (1-methylpyrrolidin-3-yl)methanamine (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (19.4 mg, 0.05 mmol, 42%). LCMS (System A): t_{RET}=0.50 min; MH⁺ 420.

### Example 88: rac-1-benzyl-2-(1,5-dimethyl-6-oxo-1,6-dihydropyridin-3-yl)-N-((tetrahydro-2H-pyran-3-yl)methyl)-1H-imidazole-4-carboxamide

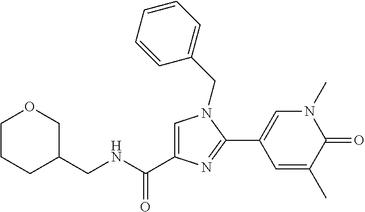 [SMILES: Cc1cc(-c2nc(C(=O)NCC3CCCOC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 132 mg, 0.40 mmol) and HATU (152 mg, 0.40 mmol) was prepared in DMF (2 mL), to which was added DIPEA (0.22 mL, 1.26 mmol). The solution was shaken and 0.5 mL was transferred to a vial containing (tetrahydro-2H-pyran-3-yl)methanamine hydrochloride salt (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (6.8 mg, 0.02 mmol, 15%). LCMS (System A): t_{RET}=0.79 min; MH⁺ 421.

### Example 89: 1-benzyl-2-(1,5-dimethyl-6-oxo-1,6-dihydropyridin-3-yl)-N-((1-methylpiperidin-4-yl)methyl)-1H-imidazole-4-carboxamide

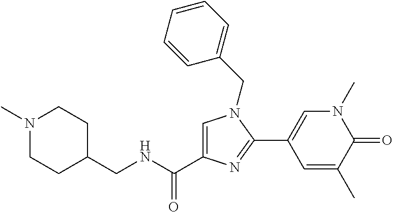 [SMILES: Cc1cc(-c2nc(C(=O)NCC3CCN(C)CC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 132 mg, 0.40 mmol) and HATU (152 mg, 0.40 mmol) was prepared in DMF (2 mL), to which was added DIPEA (0.22 mL, 1.26 mmol). The solution was shaken and 0.5 mL was transferred to a vial containing (1-methylpiperidin-4-yl)methanamine (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (18.5 mg, 0.04 mmol, 38%). LCMS (System A): t_{RET}=0.50 min; MH⁺ 434.

### Example 90: N-(2-(4-aminopiperidin-1-yl)ethyl)-1-benzyl-2-(1,5-dimethyl-6-oxo-1,6-dihydropyridin-3-yl)-1H-imidazole-4-carboxamide

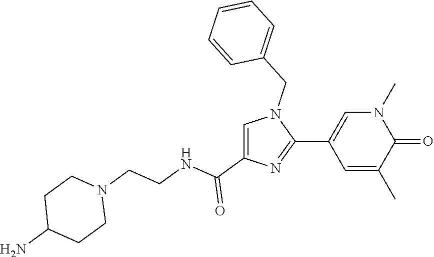 [SMILES: Cc1cc(-c2nc(C(=O)NCCN3CCC(N)CC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing tert-butyl (1-(2-aminoethyl)piperidin-4-yl)carbamate (0.12 mmol). The vial was capped and stood at RT for 2 h. The solvent was removed and the sample redissolved in DMSO (0.5 mL) and purified by MDAP (Method B). The solvent was removed under a stream of nitrogen and the sample dissolved in a solution of DCM (0.5 mL) and HCl in 1,4-dioxane (4M, 0.5 mL). The solution was capped and stood at RT for 1 h, after which the solvent was removed to afford the title compound as the dihydrochloride salt (25.0 mg, 0.05 mmol, 43%). LCMS (System A): t_{RET}=0.37 min; MH⁺ 449.

### Example 91: 1-benzyl-2-(1,5-dimethyl-6-oxo-1,6-dihydropyridin-3-yl)-N-(2-(4-methylpiperazin-1-yl)ethyl)-1H-imidazole-4-carboxamide

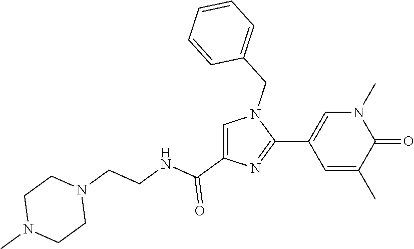 [SMILES: Cc1cc(-c2nc(C(=O)NCCN3CCN(C)CC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing 2-(4-methylpiperazin-1-yl)ethanamine (0.12 mmol). The vial was capped and stood at RT for 2 h. The solvent was removed and the sample redissolved in DMSO (0.5 mL) and purified by MDAP (Method B). The solvent was removed under a stream of nitrogen to afford the title compound (11.5 mg, 0.03 mmol, 23%). LCMS (System A): t_{RET}=0.46 min; MH⁺ 449.

### Example 92: N-(((1r,4r)-4-aminocyclohexyl)methyl)-1-benzyl-2-(1,5-dimethyl-6-oxo-1,6-dihydropyridin-3-yl)-1H-imidazole-4-carboxamide

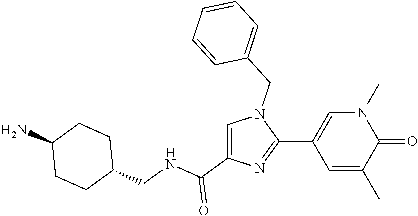 [SMILES: Cc1cc(-c2nc(C(=O)NC[C@H]3CC[C@H](N)CC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing tert-butyl ((1r,4r)-4-(aminomethyl)cyclohexyl)carbamate (0.12 mmol). The vial was capped and stood at RT for 2 h. The solvent was removed and the sample redissolved in DMSO (0.5 mL) and purified by MDAP (Method B). The solvent was removed under a stream of nitrogen and the sample dissolved in a solution of DCM (0.5 mL) and 4M HCl in 1,4-dioxane (0.5 mL). The solution was capped and stood at RT for 1 h, after which the solvent was removed to afford the title compound as the hydrochloride salt (33.5 mg, 0.07 mmol, 64%). LCMS (System A): t_{RET}=0.52 min; MH⁺ 434.

### Example 93: rac-1-benzyl-2-(1,5-dimethyl-6-oxo-1,6-dihydropyridin-3-yl)-N-(2-(piperidin-1-yl)propyl)-1H-imidazole-4-carboxamide

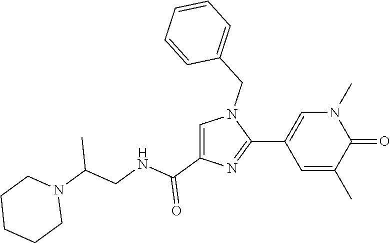 [SMILES: Cc1cc(-c2nc(C(=O)NCC(C)N3CCCCC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.8 mmol). The solution was shaken and 0.5 mL was transferred to a vial containing 2-(piperidin-1-yl)propan-1-amine (0.12 mmol). The vial was capped and stood at RT for 2 h. Propylphosphonic anhydride solution (50% w/w in EtOAc) (0.12 mL, 0.20 mmol) and further DIPEA (55 μL, 0.32 mmol) were added to the vial, which was capped, shaken, and stood at RT for 1 h. The solvent was removed and the sample redissolved in DMSO (0.5 mL) and purified by MDAP (Method B). The solvent was removed under a stream of nitrogen to afford the title compound (1.3 mg, 2.9 μmol, 3%). LCMS (System A): t_{RET}=0.53 min; MH⁺ 448.

### Example 94: 1-benzyl-2-(1,5-dimethyl-6-oxo-1,6-dihydropyridin-3-yl)-N-(2-(4-hydroxypiperidin-1-yl)-2-oxoethyl)-1H-imidazole-4-carboxamide

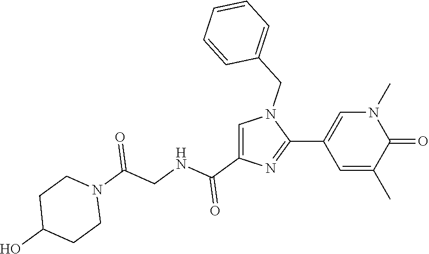 [SMILES: Cc1cc(-c2nc(C(=O)NCC(=O)N3CCC(O)CC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 16, 395 mg, 1.20 mmol) and HATU (456 mg, 1.20 mmol) was prepared in DMF (6 mL), to which was added DIPEA (0.66 mL, 3.80 mmol). The solution was shaken and 0.5 mL was transferred to a vial containing 2-amino-1-(4-hydroxpiperidin-1-yl)ethanone (0.12 mmol). The vial was capped and stood at RT for 2 h. The solvent was removed and the sample redissolved in DMSO (0.5 mL) and purified by MDAP (Method B). The solvent was removed under a stream of nitrogen and the resulting sample dissolved in DMSO (1 mL) before repurification by MDAP (Method A) The solvent was removed in vacuo and the resulting sample redissolved in 1:1 DMSO:MeOH (0.9 mL) and repurified by MDAP (Method A) to afford the title compound (3.0 mg, 6.5 μmol, 6%). LCMS (System B): t_{RET}=0.71 min; MH⁺ 464.

### Example 95: 4-((1-benzyl-2-(1,5-dimethyl-6-oxo-1,6-dihydropyridin-3-yl)-1H-imidazole-4-carboxamido)methyl)cyclohexanecarboxylic acid

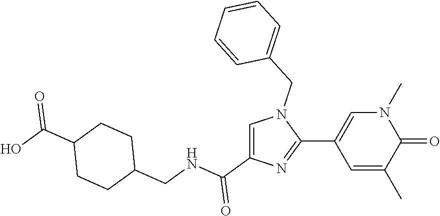 [SMILES: Cc1cc(-c2nc(C(=O)NCC3CCC(C(=O)O)CC3)cn2Cc2ccccc2)cn(C)c1=O]

A stock solution of lithium 1-benzyl-2-(1,5-dimethyl-6-oxo-1,6-dihydropyridin-3-yl)-1H-imidazole-4-carboxylate (for an example preparation, see Intermediate 13, 395 mg, 1.20 mmol) and HATU (456 mg, 1.20 mmol) was prepared in DMF (6 mL), to which was added DIPEA (0.66 mL, 3.80 mmol). The solution was shaken and 0.5 mL was transferred to a vial containing tert-butyl 4-(aminomethyl)cyclohexanecarboxylate (26 mg, 0.12 mmol). The vial was capped and stood at RT for 2 h. Propylphosphonic anhydride solution (50% w/w in EtOAc) (0.12 mL, 0.20 mmol) and further DIPEA (55 μL, 0.32 mmol) were added to the vial, which was capped, shaken, and stood at RT for 1 h. The solvent was removed and the sample redissolved in DMSO (0.5 mL) and purified by CAT MDAP (Method B). The solvent was removed under a stream of nitrogen and the sample dissolved in a solution of DCM (0.5 mL) and HCl in 1,4-dioxane (4M, 0.5 mL). The solution was capped and stood at RT for 1 h, after which the solvent was removed. The sample was redissolved in a solution of DCM (0.5 mL) and 4M HCl in 1,4-dioxane (0.5 mL). The solution was capped and stirred at 40° C. for 1 h, after which the solvent was removed. The sample was redissolved in a solution of DCM (0.5 mL) and 4M HCl in 1,4-dioxane (0.5 mL). The solution was capped and stood at RT for 20 h, after which the solvent was removed. The resulting sample was dissolved in DMSO (1 mL) before purification by MDAP (Method A). The solvent was removed in vacuo and the resulting sample redissolved in 1:1 DMSO:MeOH (0.9 mL) and repurified by MDAP (Method B). The solvent was removed under a stream of nitrogen to afford the title compound (0.9 mg, 1.9 μmol, 2%). LCMS (System B): t_{RET}=0.62 min; MH⁺ 463.

### Example 96: 5-(1-(3-(2-hydroxyethyl)benzyl)-1H-imidazol-2-yl)-1,3-dimethylpyridin-2(1H)-one

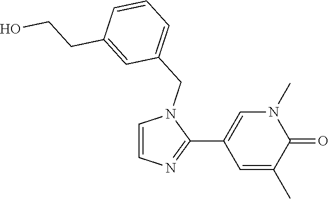 [SMILES: Cc1cc(-c2nccn2Cc2cccc(CCO)c2)cn(C)c1=O]

Methyl 2-(3-((2-(1,5-dimethyl-6-oxo-1,6-dihydropyridin-3-yl)-1H-imidazol-1-yl)methyl)phenyl)acetate (for an example preparation, see Intermediate 25, 3.00 g, 7.78 mmol) was added to THF (26 mL) and methanol (8.67 mL) and the solution cooled to 0° C. Lithium borohydride (0.678 g, 31.1 mmol) was added portionwise to the reaction mixture over 10 min. The resulting mixture was stirred at RT for 16 h, then cooled to 0° C. and quenched with saturated ammonium chloride solution (25 mL). The organic phase was separated and the aqueous layer extracted with 10% MeOH in DCM (2×100 mL). The organic layers were combined and dried over sodium sulfate, then filtered and the solvent removed from the filtrate under reduced pressure. The crude sample was purified by flash column chromatography using an elution gradient of 0-15% MeOH in DCM (100 g silica cartridge). The appropriate fractions were combined and the solvent removed in vacuo to afford the title compound as an off-white gum (1.3 g, 3.78 mmol, 49%). LCMS (System E): t_{RET}=2.15 min; MH⁺ 324.

### Example 97: 5-(1-benzyl-1H-imidazol-5-yl)-1,3-dimethylpyridin-2(1H)-one

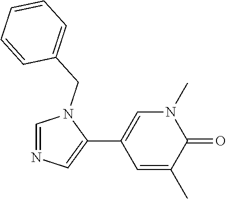 [SMILES: Cc1cc(-c2cncn2Cc2ccccc2)cn(C)c1=O]

A mixture of 5-bromo-1,3-dimethylpyridin-2(1H)-one (100 mg, 0.495 mmol), 1-benzyl-5-(4,4,5,5-tetramethyl-1,3,2-dioxaborolan-2-yl)-1H-imidazole (commercially available from, for example, J & W PharmLab, 211 mg, 0.742 mmol), potassium carbonate (342 mg, 2.47 mmol) and bis(triphenylphosphine)palladium(II) chloride (35 mg, 0.05 mmol) in EtOH (2 mL) and toluene (2 mL) was heated in a microwave at 120° C. for 1 h. The cooled reaction mixture was diluted with ethyl acetate (25 mL). The mixture was filtered and the solvent was evaporated from the filtrate. The residue was chromatographed [0-10% 2 M NH₃/methanol in dichloromethane] to give the title compound as a colourless oil (31 mg). LCMS (System B): t_{RET}=0.71 min; MH⁺ 280.

### Example 98: 5-(1-benzyl-4-chloro-1H-imidazol-5-yl)-1,3-dimethylpyridin-2(1H)-one

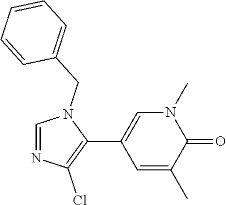 [SMILES: Cc1cc(-c2c(Cl)ncn2Cc2ccccc2)cn(C)c1=O]

1,3-Dimethyl-5-(4,4,5,5-tetramethyl-1,3,2-dioxaborolan-2-yl)pyridin-2(1H)-one (11.2 mg, 0.045 mmol), 1-benzyl-5-bromo-4-chloro-1H-imidazole (for an example preparation, see Intermediate 26, 10.2 mg, 0.038 mmol), and potassium carbonate (12.9 mg, 0.094 mmol) were added to a microwave vial containing a stirrer bar. 1,4-Dioxane (0.4 mL) and Water (0.13 mL) were added to the vial, which was purged with nitrogen for 5 min prior to the addition of tetrakis(triphenylphosphine)palladium(0) (1.3 mg, 1.1 μmol). After a further 5 min purge with nitrogen, the vial was capped and heated in the microwave at 110° C. for 45 min. The solvent removed from the reaction mixture under reduced pressure, and the residue stirred to form a suspension in ethyl acetate. This was filtered through celite and rinsed through with further ethyl acetate, the solvent again removed under reduced pressure. The sample was dissolved in 1:1 MeOH:DMSO 0.9 mL and purified by MDAP (Method B). The solvent was dried under a stream of nitrogen to give the title compound as a white solid (5.8 mg). LCMS (System B): t_{RET}=0.84 min; MH⁺ 314, 316.

### Example 99: 1-(1-benzyl-1H-imidazol-2-yl)-3,5-dimethylpyridin-4(1H)-one

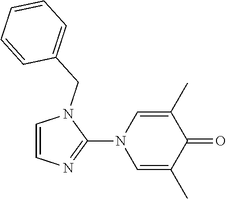 [SMILES: Cc1cn(-c2nccn2Cc2ccccc2)cc(C)c1=O]

Copper(I) iodide (4 mg, 0.021 mmol), potassium carbonate (58.3 mg, 0.422 mmol), 3,5-dimethylpyridin-4-ol (31.2 mg, 0.253 mmol), 1-benzyl-2-bromo-1H-imidazole (50 mg, 0.211 mmol) and 2,2,6,6-tetramethyl-3,5-heptanedione (0.018 mL, 0.084 mmol) were dissolved in DMSO (0.5 mL) in a microwave vial and the solvent purged and backfilled with nitrogen three times. The reaction was heated to 110° C. in an oil bath for 24 h. The reaction was diluted with MeOH (0.4 mL) and filtered through a syringe filter. Purification by MDAP (Method B) afforded the title compound as a colourless film (6.5 mg). LCMS (System B): t_{RET}=0.75 min; MH⁺ 280, 281.

### Example 100: tert-butyl (1-benzyl-2-(1,5-dimethyl-6-oxo-1,6-dihydropyridin-3-yl)-1H-imidazol-4-yl)carbamate

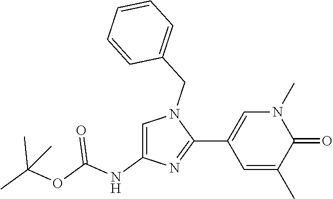 [SMILES: Cc1cc(-c2nc(NC(=O)OC(C)(C)C)cn2Cc2ccccc2)cn(C)c1=O]

5-(1-Benzyl-4-bromo-1H-imidazol-2-yl)-1,3-dimethylpyridin-2(1H)-one (100 mg, 0.279 mmol), tert-butyl carbamate (164 mg, 1.396 mmol), sodium tert-butoxide (53.7 mg, 0.558 mmol), tBuBrettPhos Pd G3 (23.85 mg, 0.028 mmol) and tBuBrettPhos (13.53 mg, 0.028 mmol) were combined in 1,4-Dioxane (2.5 mL). The reaction was heated at 110° C. for 16 h. Further tBuBrettPhos Pd G3 (23.85 mg, 0.028 mmol), tBuBrettPhos (13.53 mg, 0.028 mmol), sodium tert-butoxide (53.7 mg, 0.558 mmol) and tert-butyl carbamate (164 mg, 1.396 mmol) were added and the reaction reheated for a further 10 h. The solvent was removed in vacuo, the crude residue redissolved in DMSO/MeOH, and the sample purified by MDAP (Method B) to afford the title compound as a colourless solid (34 mg). LCMS (System B): t_{RET}=1.04 min; MH⁺ 395.

### Biological Data

### Time Resolved Fluorescence Resonance Energy Transfer (TR-FRET) Assay

Bromodomain binding was assessed utilising a time resolved fluorescent resonance energy transfer (TR-FRET) competition assay. To enable this approach a known, high affinity, pan-BET interacting small molecule was labeled with Alexa Fluor® 647, which is a far-red-fluorescent dye (Reference Compound X). Reference Compound X acts as a reporter of bromodomain binding and is the acceptor fluorophore component of the TR-FRET pair. Europium chelate, conjugated to an anti-6*His antibody, was utilised as the donor fluorophore in the TR-FRET pair (PerkinElmer AD0111). The anti-6*His antibody binds selectively to a six Histidine purification epitope added to the amino-terminus of each of the BET tandem bromodomain containing protein constructs used in this study. A TR-FRET signal is generated when the donor and acceptor fluorophores are in close proximity, between 20-80 Å, which is enabled in this assay by binding of Reference Compound X to the bromodomain containing protein.

### Reference Compound X: 4-((Z)-3-(6-((5-(2-((4S)-6-(4-chlorophenyl)-8-methoxy-1-methyl-4H-benzo[f][1,2,4]triazolo[4,3-a][1,4]diazepin-4-yl)acetamido)pentyl)amino)-6-oxohexyl)-2-((2E,4E)-5-(3,3-dimethyl-5-sulfo-1-(4-sulfobutyl)-3H-indol-1-ium-2-yl)penta-2,4-dien-1-ylidene)-3-methyl-5-sulfoindolin-1-yl)butane-1-sulphonate)

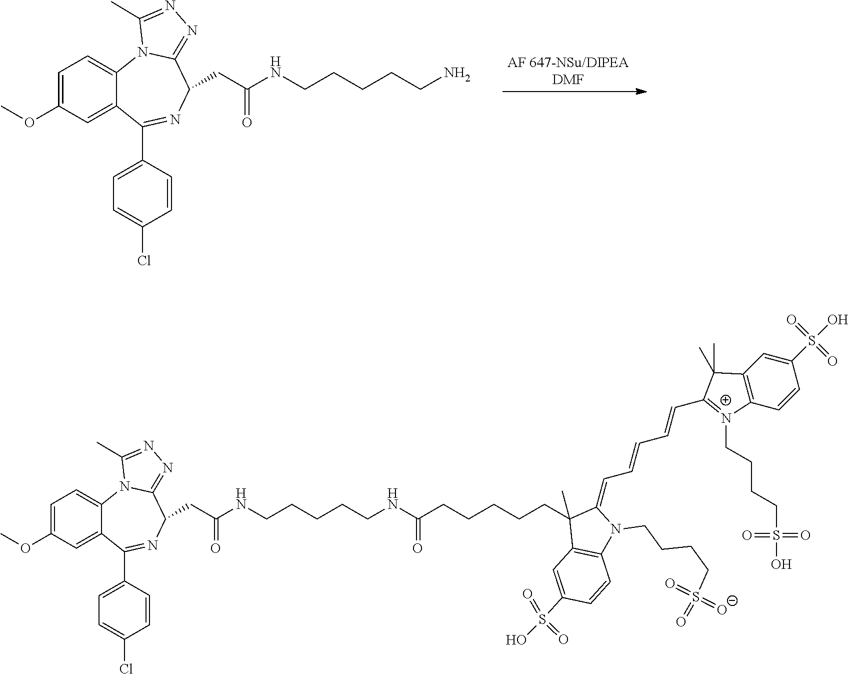

To a solution of N-(5-aminopentyl)-2-((4S)-6-(4-chlorophenyl)-8-methoxy-1-methyl-4H-benzo[f][1,2,4]triazolo[4,3-a][1,4]diazepin-4-ylacetamide (for a preparation see Reference Compound J, WO2011/054848A1, 1.7 mg, 3.53 μmol) in DMF (40 μL) was added a solution of AlexaFluor647-ONSu (2.16 mg, 1.966 μmol) also in DMF (100 μL). The mixture was basified with DIPEA (1 μl, 5.73 μmol) and agitated overnight on a vortex mixer. The reaction mixture was evaporated to dryness. The solid was dissolved in MeCN/water/AcOH (5/4/1, <1 mL) filtered and was applied to a Phenomenex Jupiter C18 preparative column and eluted with the following gradient (A=0.1% trifluoroacetic acid in water, B=0.1% TFA/90% MeCN/10% water): Flow rate=10 mL/min., AU=20/10 (214 nm): 5-35%, t=0 min: B=5%; t=10 min: B=5%; t=100 min: B=35%; t=115 min: B=100% (Sep. grad: 0.33%/min)

The major component was eluted over the range 26-28% B but appeared to be composed of two peaks. The middle fraction (F1.26) which should contain “both” components was analysed by analytical HPLC (Spherisorb ODS2, 1 to 35% over 60 min): single component eluting at 28% B. Fractions F1.25/26&27 were combined and evaporated to dryness. Transfered with DMF, evaporated to dryness, triturated with dry ether and the blue solid dried overnight at <0.2 mbar: 1.54 mg. Analytical HPLC (Sphersisorb ODS2, 1 to 35% B over 60 min): MSM10520-1: [M+H]⁺ (obs): 661.8/-corresponding with M-29. This equates to [(M+2H)/2]⁺ for a calculated mass of 1320.984 which is M-29. This is a standard occurrence with the Alexa Fluor 647 dye and represents a theoretical loss of two methylene groups under the conditions of the mass spectrometer.

Assay Principle:

In order to generate a TR-FRET signal, donor fluorophore is excited by a laser at λ337 nm, which subsequently leads to emission at λ618 nm. If the acceptor fluorophore is in close proximity then energy transfer can occur, which leads to emission of Alexa Fluor® 647 at λ665 nm. In the presence of competitor compound, Reference Compound X can be displaced from binding to the bromodomain. If displacement occurs, the acceptor fluorophore is no longer in proximity to the donor fluorophore, which prevents fluorescent energy transfer and, subsequently, a loss of Alexa Fluor® 647 emission at λ665 nm.

The competition of the compounds of formula (I) with Reference Compound X for binding to the BET family (BRD2, BRD3, BRD4 and BRDT) was assessed using protein truncates spanning both bromodomain 1 (BD1) and bromodomain 2 (BD2). In order to monitor differential binding to either BD1 or BD2, single residue mutations of key tyrosines to alanine were made in the acetyl lysine binding pockets. To validate this approach, a double residue mutant tandem domain protein was produced for each of the BET family members. Utilising a Fluorescence Polarisation approach, binding affinities for each of the single and double mutants for Reference Compound X were determined. The affinities of the double mutant tandem proteins for Reference Compound X were greatly reduced in comparison to the non mutated, wild type tandem BET proteins (>1000 fold reduction in Kd). The affinities of the single mutated bromodomain tandem proteins for Reference Compound X were equi-potent with the corresponding non-mutated BET protein. These data demonstrated that single mutations of Tyrosine to Alanine reduce the Kd of the interaction between the mutated bromodomain and Reference Compound X by >1000 fold. In the TR-FRET competition assay, Reference Compound X is used at a concentration that is equivalent to the Kd for the non-mutated bromodomain, which ensures that no binding at the mutated bromodomain is detected.

Protein Production:

Recombinant Human Bromodomains [(BRD2 (1-473) (Y113A) and (Y386A), BRD3 (1-435) (Y73A) and (Y348A) BRD4 (1-477) (Y97A) and (Y390A) and BRDT (1-397) (Y66A) and (Y309A)] were expressed in E. coli cells (in pET15b vector for BRD2/3/4 and in pET28a vector for BRDT) with a 6-His tag at the N-terminal. The His-tagged Bromodomain pellet was resuspended in 50 mM HEPES (pH7.5), 300 mM NaCl, 10 mM imidazole & 1 μL/mL protease inhibitor cocktail and extracted from the E. coli cells using sonication and purified using a nickel sepharose high performance column, the proteins were washed and then eluted with a linear gradient of 0-500 mM imidazole with buffer 50 mM HEPES (pH7.5), 150 mM NaCl, 500 mM imidazole, over 20 column volumes. Final purification was completed by Superdex 200 prep grade size exclusion column. Purified protein was stored at −80° C. in 20 mM HEPES pH 7.5 and 100 mM NaCl. Protein identity was confirmed by peptide mass fingerprinting and predicted molecular weight confirmed by mass spectrometry.

Protocol for Bromodomain BRD2, 3, 4 and T, BD1+BD2 Mutant TR-FRET Competition Assays:

All assay components were dissolved in an assay buffer composing of 50 mM HEPES pH7.4, 50 mM NaCl, 5% Glycerol, 1 mM DTT and 1 mM CHAPS. Reference Compound X was diluted, in assay buffer containing 20 nM single mutant, tandem bromodomain containing protein, to a concentration equivalent to 2*Kd for this bromodomain. The solution containing bromodomain and Reference Compound X was added to dose response dilutions of test compound or DMSO vehicle (a maximum of 0.5% DMSO is used in this assay) in Greiner 384 well black low volume microtitre plates and subsequently incubated for 30 minutes at RT. An equal volume of 3 nM of anti-6*His Europium chelate was added to all wells, followed by a further 30 minute incubation at room temperature. TR-FRET was detected using a Perkin Elmer Multimode plate reader, by exciting the donor fluorophore at λ337 nm and subsequently, after a delay of 50 μsecs, measuring emission of the donor and acceptor fluorophores at λ615 nm and λ665 nm, respectively. In order to control these assays, 16 wells each of uninhibited (DMSO vehicle) and inhibited (10*IC₅₀ concentrations of Example 11 of WO 2011/054846A1) reactions were included on every microtitre plate.

A four parameter curve fit of the following form was then applied:

y=a+((b−a)/(1+(10\hat{ }x/10\hat{ }c)\hat{ }d)

Where “a” is the minimum, “b” is the Hill slope, “c” is the pIC₅₀ and “d” is the maximum.

Results:

All the Examples were tested in the above BRD4 assay and were found to have a mean pIC₅₀ in the range of 5.5 to 7.9 in the BRD4 BD1 assay and a mean pIC₅₀ in the range of 4.6 to 7.0 in the BRD4 BD2 assay.

Measurement of LPS Induced MCP-1 Production from Human Whole Blood

Activation of monocytic cells by agonists of toll-like receptors such as bacterial lipopolysaccharide (LPS) results in production of key inflammatory mediators including MCP-1. Such pathways are widely considered to be central to the pathophysiology of a range of auto-immune and inflammatory disorders.

Blood is collected in a tube containing Sodium heparin (Leo Pharmaceuticals) (10 units of heparin/mL of blood). 96-well compound plates containing 0.5 μL test sample (compound) in 100% DMSO were prepared (two replicates on account of donor variability). 130 μL of whole blood was dispensed into each well of the 96-well compound plates and incubated for 30 min at 37° C., 5% CO₂. 10 μL of lipopolysaccharide (from Salmonella typhosa; L6386) made up in PBS (200 ng/mL final assay concentration) was added to each well of the compound plates. The plates were then placed in the humidified primary cell incubator for 18-24 hours at 37° C., 5% CO₂. 140 μL of PBS was added to all wells of the compound plates containing blood. The plates were then sealed and centrifuged for 10 mins at 2500 rpm (r.t). 20 μL of cell supernatant was placed in a 96-well MSD plate pre-coated with human MCP-1 capture antibody. The plates were sealed and placed on a shaker at 600 rpm for 1.5 hour (RT). 20 μL of Anti-human MCP-1 antibody labelled with MSD SULFO-TAG™ reagent is added to each well of the MSD plate (stock 50× was diluted 1:50 with Diluent 100, final assay concentration is 1 μg/mL). The plates were then re-sealed and shaken for 1 hour (RT) before washing with 3× with PBS Tween 0.05%. 150 μL of 2×MSD Read Buffer T (stock 4×MSD Read Buffer T was diluted 50:50 with de-ionised water) was then added to each well and the plates read on the MSD Sector Imager 6000. Concentration response curves for each compound were generated from the data and an IC₅₀ value was calculated.

Results:

All the Examples, with the exception of Examples 2, 3, 35, 65, 66, 95, 96, 97) were tested in the above assay and were found to have a mean pIC₅₀ in the range of 5.0 to 6.8. Example 34 had a mean pIC50 of <4.7. These data demonstrate that bromodomain inhibitors of the present invention inhibit the production of the key inflammatory mediator MCP-1 in the above assay.

1. A compound of formula (I), or a salt thereof: 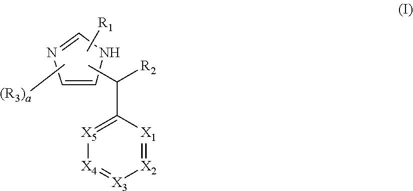
wherein
R₁ represents 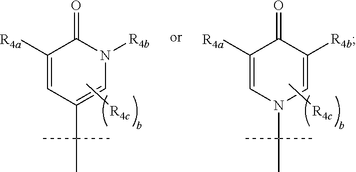
R₂ is hydrogen or C₁₋₃alkyl;
each R₃ is independently selected from the group consisting of halogen, —CN, —C₁₋₃alkyl, C₁₋₃alkoxy, —NO₂, —CONR₅R₆, —NR₅COR₆, —NR₅CO₂R₆, —OCOR₆, —CO₂R₆, —SO₂NR₅R₆, —NR₅SO₂R₆, —SO₂R₆, —R₆, —NR₅R₆, and —OR₆, with the proviso that when a is 2, one R₃ is selected from the group consisting of halogen, —CN, —C₁₋₃alkyl and C₁₋₃alkoxy;
R₄ₐ is hydrogen, C₁₋₃alkyl, C₁₋₃alkoxy, halogen, —CN, —OH, or —NR₇R₈;
R_{4b} is hydrogen or C₁₋₃alkyl;
each R_{4c} is independently selected from the group consisting of C₁₋₃alkyl, C₁₋₃alkoxy, halogen, —CN, —OH, and —NR₇R₈;
R₅ is hydrogen or C₁₋₃alkyl and R₆ is —Y—Z, or when R₃ is —CONR₅R₆, R₅ and R₆ together with the nitrogen to which they are attached may form a heterocycloalkyl, wherein the heterocycloalkyl group can be optionally substituted with one or two groups independently selected from C₁₋₃alkyl, halogen, —NH₂, —CH₂NH₂, —CO₂H, —OH, —CN, and —CH₂OH;
Y is a bond or C₁₋₃alkylene, wherein the C₁₋₃alkylene group can be optionally substituted with one or two groups independently selected from C₁₋₃alkyl and ═O;
Z is hydrogen, C₁₋₃alkyl, cycloalkyl, heterocycloalkyl, aryl, heteroaryl, —SO₂NR₉R₁₀, —NR₉SO₂R₁₀, —SO₂R₉, or —NR₉R₁₀, wherein the C₁₋₃alkyl, cycloalkyl, heterocycloalkyl, aryl or heteroaryl group can be optionally substituted with one or two groups independently selected from C₁₋₃alkyl, C₁₋₃alkoxy, halogen, —NH₂, —CH₂NH₂, —CO₂H, —C(O)CH₃, —OH, —CN, and —CH₂OH;
X₁, X₂, X₃, X₄ and X₅ are each independently selected from CR₁₁ and N with the proviso that zero, one or two of X₁, X₂, X₃, X₄ and X₅ are N;
each R₇ is independently selected from hydrogen or CH₃;
each R₈ is independently selected from hydrogen or C₁₋₃alkyl;
R₉ is hydrogen or C₁₋₃alkyl;
R₁₀ is hydrogen or C₁₋₃alkyl;
or when Z is —SO₂NR₉R₁₀, R₉ and R₁₀ together with the nitrogen to which they are attached may form a heterocycloalkyl;
each R₁₁ is independently selected from hydrogen, C₁₋₃alkyl, C₁₋₃alkoxy, halogen, —CN, —OH, —OCF₃, —OCHF₂, —SO₂C₁₋₃alkyl, —CH₂OH, —CH₂CH₂OH, and CF₃;
or any two R₁₁ groups on adjacent carbon atoms together with the atoms to which they are attached may form a 5- or 6-membered heteroaryl group;
a is 0, 1 or 2; and
b is 0, 1 or 2.

2-3. (canceled)

4. The compound according to claim 1 comprising a compound of formula (Ia), or a salt thereof: 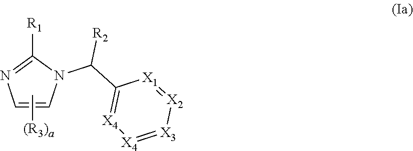
wherein R₁, R₂, R₃, a, X₁, X₂, X₃, X₄ and X₅ are as defined in claim 1.

5. (canceled)

6. The compound or salt according to claim 1, wherein R₁ represents

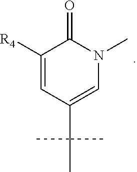

7. The compound or salt according to claim 1, wherein R₄ₐ is CH₃ or —OCH₃.

8. (canceled)

9. The compound or salt according to claim 1, wherein R_{4b} is CH₃.

10. The compound of salt according to claim 1, wherein b is 0.

11. The compound or salt according to claim 1, wherein R₂ is hydrogen or CH₃.

12. (canceled)

13. The compound or salt according to claim 1, wherein a is 1.

14. (canceled)

15. The compound or salt according to claim 1, wherein each R₃ is independently selected from the group consisting of halogen, —CN, —C₁₋₃alkyl, and C₁₋₃alkoxy.

16-21. (canceled)

22. The compound or salt according to claim 1, wherein X₁, X₂, X₃, X₄ and X₅ each represent CR₁₁.

23. The compound or salt according to claim 1, wherein one or two of X₁, X₂, X₃, X₄ and X₅ represent N.

24. The compound or salt according to claim 1, wherein one of X₁, X₂, X₃, X₄ and X₅ represent N.

25. The compound or salt according to claim 1, wherein X₁ and X₅, or X₂ and X₄ represent N.

26-27. (canceled)

28. The compound according to claim 1, which is selected from the group consisting of:
5-(1-benzyl-1H-imidazol-2-yl)-1,3-dimethylpyridin-2(1H)-one;
1,3-dimethyl-5-(1-(pyridin-3-ylmethyl)-1H-imidazol-2-yl)pyridin-2(1H)-one;
1,3-dimethyl-5-(1-(pyridin-4-ylmethyl)-1H-imidazol-2-yl)pyridin-2(1H)-one;
1,3-dimethyl-5-(1-(pyridin-2-ylmethyl)-1H-imidazol-2-yl)pyridin-2(1H)-one;
1,3-dimethyl-5-(1-(quinolin-8-ylmethyl)-1H-imidazol-2-yl)pyridin-2(1H)-one;
4-((2-(1,5-dimethyl-6-oxo-1,6-dihydropyridin-3-yl)-1H-imidazol-1-yl)methyl)benzonitrile;
5-((2-(1,5-dimethyl-6-oxo-1,6-dihydropyridin-3-yl)-1H-imidazol-1-yl)methyl)picolinonitrile;
5-(1-(3-methoxybenzyl)-1H-imidazol-2-yl)-1,3-dimethylpyridin-2(1H)-one;
1,3-dimethyl-5-(1-(4-(methylsulfonyl)benzyl)-1H-imidazol-2-yl)pyridin-2(1H)-one;
methyl 1-benzyl-2-(1,5-dimethyl-6-oxo-1,6-dihydropyridin-3-yl)-1H-imidazole-4-carboxylate;
methyl 1-benzyl-2-(1,5-dimethyl-6-oxo-1,6-dihydropyridin-3-yl)-1H-imidazole-5-carboxylate;
5-(1-benzyl-4-chloro-1H-imidazol-2-yl)-1,3-dimethylpyridin-2(1H)-one;
1,3-dimethyl-5-(1-(1-phenylethyl)-1H-imidazol-2-yl)pyridin-2(1H)-one;
1,3-dimethyl-5-(1-(pyrimidin-2-ylmethyl)-1H-imidazol-2-yl)pyridin-2(1H)-one;
1,3-dimethyl-5-(1-(pyrimidin-5-ylmethyl)-1H-imidazol-2-yl)pyridin-2(1H)-one;
1,3-dimethyl-5-(1-(3-(trifluoromethyl)benzyl)-1H-imidazol-2-yl)pyridin-2(1H)-one;
5-(1-(2-chlorobenzyl)-1H-imidazol-2-yl)-1,3-dimethylpyridin-2(1H)-one;
1,3-dimethyl-5-(1-(4-methylbenzyl)-1H-imidazol-2-yl)pyridin-2(1H)-one;
5-(1-(3,4-dichlorobenzyl)-1H-imidazol-2-yl)-1,3-dimethylpyridin-2(1H)-one;
5-(1-(4-methoxybenzyl)-1H-imidazol-2-yl)-1,3-dimethylpyridin-2(1H)-one;
5-(1-(3,5-difluorobenzyl)-1H-imidazol-2-yl)-1,3-dimethylpyridin-2(1H)-one;
1,3-dimethyl-5-(1-(4-(trifluoromethyl)benzyl)-1H-imidazol-2-yl)pyridin-2(1H)-one;
5-(1-(2-methoxybenzyl)-1H-imidazol-2-yl)-1,3-dimethylpyridin-2(1H)-one;
5-(1-(3-chloro-5-fluoro-4-methoxybenzyl)-1H-imidazol-2-yl)-1,3-dimethylpyridin-2(1H)-one;
5-(1-(3-fluoro-4-methoxybenzyl)-1H-imidazol-2-yl)-1,3-dimethylpyridin-2(1H)-one;
(S)-5-(1-benzyl-4-(2-(hydroxymethyl)pyrrolidine-1-carbonyl)-1H-imidazol-2-yl)-1,3-dimethylpyridin-2(1H)-one;
1,3-dimethyl-5-(1-((6-methylpyridin-2-yl)methyl)-1H-imidazol-2-yl)pyridin-2(1H)-one;
5-(1-(2-fluorobenzyl)-1H-imidazol-2-yl)-1,3-dimethylpyridin-2(1H)-one;
5-(1-(4-fluorobenzyl)-1H-imidazol-2-yl)-1,3-dimethylpyridin-2(1H)-one;
1,3-dimethyl-5-(1-(4-(trifluoromethoxy)benzyl)-1H-imidazol-2-yl)pyridin-2(1H)-one;
5-(1-(4-(difluoromethoxy)benzyl)-1H-imidazol-2-yl)-1,3-dimethylpyridin-2(1H)-one;
5-(1-(2,3-dichlorobenzyl)-1H-imidazol-2-yl)-1,3-dimethylpyridin-2(1H)-one;
5-(1-(3-chlorobenzyl)-1H-imidazol-2-yl)-1,3-dimethylpyridin-2(1H)-one;
1-benzyl-2-(1,5-dimethyl-6-oxo-1,6-dihydropyridin-3-yl)-1H-imidazole-4-carboxylic acid;
1,3-dimethyl-5-(1-((5-methylpyridin-3-yl)methyl)-1H-imidazol-2-yl)pyridin-2(1H)-one;
1,3-dimethyl-5-(1-(3-methylbenzyl)-1H-imidazol-2-yl)pyridin-2(1H)-one;
5-(1-(3-fluorobenzyl)-1H-imidazol-2-yl)-1,3-dimethylpyridin-2(1H)-one;
5-(1-(4-chlorobenzyl)-1H-imidazol-2-yl)-1,3-dimethylpyridin-2(1H)-one;
1,3-dimethyl-5-(1-(pyridazin-3-ylmethyl)-1H-imidazol-2-yl)pyridin-2(1H)-one;
5-(1-benzyl-4-nitro-1H-imidazol-2-yl)-1,3-dimethylpyridin-2(1H)-one;
5-(5-chloro-1-(1-phenylethyl)-1H-imidazol-2-yl)-1,3-dimethylpyridin-2(1H)-one;
1-benzyl-2-(1,5-dimethyl-6-oxo-1,6-dihydropyridin-3-yl)-N,N-dimethyl-1H-imidazole-4-carboxamide;
5-(1-benzyl-4-(morpholine-4-carbonyl)-1H-imidazol-2-yl)-1,3-dimethylpyridin-2(1H)-one;
5-(1-benzyl-4-(pyrrolidine-1-carbonyl)-1H-imidazol-2-yl)-1,3-dimethylpyridin-2(1H)-one;
5-(4-(azetidine-1-carbonyl)-1-benzyl-1H-imidazol-2-yl)-1,3-dimethylpyridin-2(1H)-one;
1-benzyl-2-(1,5-dimethyl-6-oxo-1,6-dihydropyridin-3-yl)-N-methyl-1H-imidazole-4-carboxamide;
5-(1-benzyl-5-chloro-1H-imidazol-2-yl)-1,3-dimethylpyridin-2(1H)-one;
1-benzyl-2-(1,5-dimethyl-6-oxo-1,6-dihydropyridin-3-yl)-N-ethyl-1H-imidazole-4-carboxamide;
1-benzyl-2-(1,5-dimethyl-6-oxo-1,6-dihydropyridin-3-yl)-N-(tetrahydrofuran-3-yl)-1H-imidazole-4-carboxamide;
5-(1-benzyl-4-(piperidine-1-carbonyl)-1H-imidazol-2-yl)-1,3-dimethylpyridin-2(1H)-one;
1-benzyl-2-(1,5-dimethyl-6-oxo-1,6-dihydropyridin-3-yl)-N-(tetrahydro-2H-pyran-4-yl)-1H-imidazole-4-carboxamide;
1-benzyl-2-(1,5-dimethyl-6-oxo-1,6-dihydropyridin-3-yl)-N-phenethyl-1H-imidazole-4-carboxamide;
N,1-dibenzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-1H-imidazole-4-carbonitrile;
1-benzyl-2-(1,5-dimethyl-6-oxo-1,6-dihydropyridin-3-yl)-1H-imidazole-5-carbonitrile;
5-(1-benzyl-4-bromo-5-chloro-1H-imidazol-2-yl)-1,3-dimethylpyridin-2(1H)-one;
N-(1-benzyl-2-(1,5-dimethyl-6-oxo-1,6-dihydropyridin-3-yl)-1H-imidazol-4-yl)acetamide;
5-(4-chloro-1-(pyridin-2-ylmethyl)-1H-imidazol-2-yl)-1,3-dimethylpyridin-2(1H)-one;
1-benzyl-2-(1,5-dimethyl-6-oxo-1,6-dihydropyridin-3-yl)-1H-imidazole-4-carboxamide;
5-(4-chloro-1-(1-(pyridin-2-yl)ethyl)-1H-imidazol-2-yl)-1,3-dimethylpyridin-2(1H)-one;
5-(5-chloro-1-(1-(pyridin-2-yl)ethyl)-1H-imidazol-2-yl)-1,3-dimethylpyridin-2(1H)-one;
5-(1-benzyl-4-fluoro-1H-imidazol-2-yl)-1,3-dimethylpyridin-2(1H)-one;
5-(1-benzyl-4-bromo-1H-imidazol-2-yl)-1,3-dimethylpyridin-2(1H)-one;
5-(1-benzyl-4-methyl-1H-imidazol-2-yl)-1,3-dimethylpyridin-2(1H)-one;
5-(4-chloro-14(5-methoxypyridin-2-yl)methyl)-1H-imidazol-2-yl)-1,3-dimethylpyridin-2(1H)-one;
5-(4-chloro-1-((6-methoxypyridin-3-yl)methyl)-1H-imidazol-2-yl)-1,3-dimethylpyridin-2(1H)-one;
5-(1-benzyl-4-phenyl-1H-imidazol-2-yl)-1,3-dimethylpyridin-2(1H)-one;
1-benzyl-N-cyclopropyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-isopropyl-1H-imidazole-4-carboxamide;
1-benzyl-2-(1,5-dimethyl-6-oxo-1,6-dihydropyridin-3-yl)-N-((tetrahydrofuran-2-yl)methyl)-1H-imidazole-4-carboxamide;
5-(4-chloro-14(5-methylpyridin-2-yl)methyl)-1H-imidazol-2-yl)-1,3-dimethylpyridin-2(1H)-one;
5-(4-chloro-14(5-fluoropyridin-2-yl)methyl)-1H-imidazol-2-yl)-1,3-dimethylpyridin-2(1H)-one;
5-((4-chloro-2-(1,5-dimethyl-6-oxo-1,6-dihydropyridin-3-yl)-1H-imidazol-1-yl)methyl)picolinonitrile;
1-benzyl-2-(1,5-dimethyl-6-oxo-1,6-dihydropyridin-3-yl)-N-(2-(dimethylamino)ethyl)-1H-imidazole-4-carboxamide;
1-benzyl-2-(1,5-dimethyl-6-oxo-1,6-dihydropyridin-3-yl)-N-(2-(N-methylsulfamoyl)ethyl)-1H-imidazole-4-carboxamide;
1-benzyl-2-(1,5-dimethyl-6-oxo-1,6-dihydropyridin-3-yl)-N-(2-(pyrrolidin-1-ylsulfonyl)ethyl)-1H-imidazole-4-carboxamide;
1-benzyl-2-(1,5-dimethyl-6-oxo-1,6-dihydropyridin-3-yl)-N-(2-sulfamoylethyl)-1H-imidazole-4-carboxamide;
1-benzyl-2-(1,5-dimethyl-6-oxo-1,6-dihydropyridin-3-yl)-N-(2-(piperazin-1-yl)ethyl)-1H-imidazole-4-carboxamide;
1-benzyl-2-(1,5-dimethyl-6-oxo-1,6-dihydropyridin-3-yl)-N-(piperidin-4-ylmethyl)-1H-imidazole-4-carboxamide;
1-benzyl-2-(1,5-dimethyl-6-oxo-1,6-dihydropyridin-3-yl)-N-(pyrrolidin-3-ylmethyl)-1H-imidazole-4-carboxamide;
1-benzyl-2-(1,5-dimethyl-6-oxo-1,6-dihydropyridin-3-yl)-N-((tetrahydrofuran-3-yl)methyl)-1H-imidazole-4-carboxamide;
N-((4-(aminomethyl)cyclohexyl)methyl)-1-benzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-(2-morpholinoethyl)-1H-imidazole-4-carboxamide;
1-benzyl-2-(1,5-dimethyl-6-oxo-1,6-dihydropyridin-3-yl)-N-((1-isopropylpyrrolidin-3-yl)methyl)-1H-imidazole-4-carboxamide;
1-benzyl-2-(1,5-dimethyl-6-oxo-1,6-dihydropyridin-3-yl)-N-(morpholin-2-ylmethyl)-1H-imidazole-4-carboxamide;
1-benzyl-2-(1,5-dimethyl-6-oxo-1,6-dihydropyridin-3-yl)-N-((tetrahydro-2H-pyran-4-yl)methyl)-1H-imidazole-4-carboxamide;
1-benzyl-2-(1,5-dimethyl-6-oxo-1,6-dihydropyridin-3-yl)-N-((1-methylpyrrolidin-3-yl)methyl)-1H-imidazole-4-carboxamide;
1-benzyl-2-(1,5-dimethyl-6-oxo-1,6-dihydropyridin-3-yl)-N-((tetrahydro-2H-pyran-3-yl)methyl)-1H-imidazole-4-carboxamide;
1-benzyl-2-(1,5-dimethyl-6-oxo-1,6-dihydropyridin-3-yl)-N-((1-methylpiperidin-4-yl)methyl)-1H-imidazole-4-carboxamide;
N-(2-(4-aminopiperidin-1-yl)ethyl)-1-benzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-(2-(4-methylpiperazin-1-yl)ethyl)-1H-imidazole-4-carboxamide;
N-(((1r,4r)-4-aminocyclohexyl)methyl)-1-benzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-N-(2-(piperidin-1-yl)propyl)-1H-imidazole-4-carboxamide;
1-benzyl-2-(1,5-dimethyl-6-oxo-1,6-dihydropyridin-3-yl)-N-(2-(4-hydroxypiperidin-1-yl)-2-oxoethyl)-1H-imidazole-4-carboxamide;
4-((1-benzyl-2-(1,5-dimethyl-6-oxo-1,6-dihydropyridin-3-yl)-1H-imidazole-4-carboxamido)methyl)cyclohexanecarboxylic acid;
5-(1-(3-(2-hydroxyethyl)benzyl)-1H-imidazol-2-yl)-1,3-dimethylpyridin-2(1H)-one;
5-(1-benzyl-1H-imidazol-5-yl)-1,3-dimethylpyridin-2(1H)-one;
5-(1-benzyl-4-chloro-1H-imidazol-5-yl)-1,3-dimethylpyridin-2(1H)-one;
1-(1-benzyl-1H-imidazol-2-yl)-3,5-dimethylpyridin-4(1H)-one; and
tert-butyl (1-benzyl-2-(1,5-dimethyl-6-oxo-1,6-dihydropyridin-3-yl)-1H-imidazol-4-yl)carbamate;
or a salt thereof.

29. The compound according to claim 1, which is selected from the group consisting of:
5-(1-benzyl-4-bromo-5-chloro-1H-imidazol-2-yl)-1,3-dimethylpyridin-2(1H)-one;
5-(1-benzyl-4-chloro-1H-imidazol-2-yl)-1,3-dimethylpyridin-2(1H)-one;
5-(1-benzyl-4-bromo-1H-imidazol-2-yl)-1,3-dimethylpyridin-2(1H)-one;
5-(1-benzyl-4-phenyl-1H-imidazol-2-yl)-1,3-dimethylpyridin-2(1H)-one;
Methyl 1-benzyl-2-(1,5-dimethyl-6-oxo-1,6-dihydropyridin-3-yl)-1H-imidazole-5-carboxylate;
5-(5-chloro-1-(1-phenylethyl)-1H-imidazol-2-yl)-1,3-dimethylpyridin-2(1H)-one;
1-benzyl-2-(1,5-dimethyl-6-oxo-1,6-dihydropyridin-3-yl)-1H-imidazole-4-carbonitrile;
5-(1-benzyl-4-methyl-1H-imidazol-2-yl)-1,3-dimethylpyridin-2(1H)-one;
Methyl 1-benzyl-2-(1,5-dimethyl-6-oxo-1,6-dihydropyridin-3-yl)-1H-imidazole-4-carboxylate;
5-(1-(4-chlorobenzyl)-1H-imidazol-2-yl)-1,3-dimethylpyridin-2(1H)-one;
1-benzyl-2-(1,5-dimethyl-6-oxo-1,6-dihydropyridin-3-yl)-N-phenethyl-1H-imidazole-4-carboxamide;
N,1-dibenzyl-2-(1,5-dimethyl-6-oxo-1,6-dihydropyridin-3-yl)-1H-imidazole-4-carboxamide;
1-benzyl-2-(1,5-dimethyl-6-oxo-1,6-dihydropyridin-3-yl)-1H-imidazole-4-carboxamide;
5-(4-chloro-1-(1-(pyridin-2-yl)ethyl)-1H-imidazol-2-yl)-1,3-dimethylpyridin-2(1H)-one;
5-(1-benzyl-4-fluoro-1H-imidazol-2-yl)-1,3-dimethylpyridin-2(1H)-one; and
5-(4-chloro-14(5-methylpyridin-2-yl)methyl)-1H-imidazol-2-yl)-1,3-dimethylpyridin-2(1H)-one;
or a salt thereof.

30. The compound according to claim 1, which is in the form of a pharmaceutically acceptable salt.

31. The compound according to claim 1, which is in the form of a free base.

32. A pharmaceutical composition comprising the compound or salt according to claim 1, and one or more pharmaceutically acceptable excipients.

33-35. (canceled)

36. A method of treatment of an autoimmune or inflammatory disease or cancer, which method comprises administering to a human subject in need thereof, a therapeutically effective amount of the compound or salt according to claim 1.

37. A method of treatment of rheumatoid arthritis, which method comprises administering to a human subject in need thereof, a therapeutically effective amount of the compound or salt according to claim 1.
